(19) United States
(12) Patent Application Publication
(10) Pub. No.: US 2024/0034999 A1
(43) Pub. Date: Feb. 1, 2024

(54) IMPROVED REPROGRAMMING, MAINTENANCE AND PRESERVATION FOR INDUCED PLURIPOTENT STEM CELLS

(71) Applicant: Fate Therapeutics, Inc., San Diego, CA (US)

(72) Inventors: Yi-Shin LAI, San Diego, CA (US); Bahram VALAMEHR, San Diego, CA (US); Ramzey ABUJAROUR, San Diego, CA (US); Hui-Ting HSU, San Diego, CA (US)

(21) Appl. No.: 18/246,174

(22) PCT Filed: Oct. 1, 2021
(86) PCT No.: PCT/US2021/053240

§ 371 Date: Mar. 21, 2023

Related U.S. Application Data
(60) Provisional application No. 63/087,119, filed on Oct. 2, 2020.

(51) Int. Cl. C12N 5/074; A61K 35/545; C12N 15/86

(52) U.S. Cl. CPC C12N 5/0696; A61K 35/545; C12N 15/86; C12N 2501/15; C12N 2501/415; C12N 2506/11; C12N 2510/00; C12N 2501/999; C12N 2501/16; C12N 2710/16243

(57) ABSTRACT

Provided are methods and compositions for inducing the reprogramming of a non-pluripotent cell using a small molecule supported vector system to provide an iPSC having desirable properties with a high efficiency. Also provided are reprogramming cells and iPSC populations or clonal cell lines using the provided reprogramming methods and compositions. Further provided are compositions and methods for maintaining and preserving iPSCs while achieving genomic stability of the cells.

### RELATED APPLICATION

This application claims priority to U.S. Provisional Application Ser. No. 63/087,119, filed Oct. 2, 2020, the disclosure of which is hereby incorporated by reference in its entirety.

### FIELD OF THE INVENTION

The present disclosure is broadly concerned with the field of generating human induced pluripotent stem cells (iPSCs or iPS cells). More particularly, the present disclosure is concerned with the use of combinations of plasmid vectors to obtain footprint-free iPSCs having desirable properties with a higher efficiency and increased reliability.

### BACKGROUND OF THE INVENTION

iPSCs were originally generated using integrating viral systems to express key transcription factors. Retroviral and lentiviral systems including polycistronic and inducible systems have now been successfully employed in iPSC generation. However, permanent genomic changes due to insertional mutagenesis and the potential for exogenous gene reactivation post iPSC differentiation may present potential problems for subsequent drug screening and therapeutic applications of cells generated by these methods. Indeed, significant differences between iPSC clones generated using the same viral systems have been reported, with a large percentage of clones forming tumors in rodents when transplanted as differentiated neurospheres. Research suggests that iPSCs generated using the same viral methods may behave differently once differentiated. Differences in ectopic gene integration site may result in different insertional mutagenesis and epigenetic regulation of transgene expression. For iPSC generation methods that include integrating systems, many clones may need to be derived and screened to identify those that are stable in both pluripotent and differentiated states.

Among the many key aspects in cell therapy manufacturing processes, cell expansion and cryopreservation have been identified as critical areas of interest, where cell viability and functionality are profoundly impacted during the freeze-thaw cycle, and in vivo cell efficacy and persistency of effector cells derived from iPSC differentiation are intricately affected during effector cell expansion stage after iPSC differentiation.

### SUMMARY OF THE INVENTION

In view of the foregoing, there is a substantial need in the art for an efficient production of a homogenous population of footprint-free iPSCs, preferably in a “naïve” or “grounded” state of pluripotency, and preferably in defined culture conditions. The “naïve” or “grounded” state of pluripotency imparts the iPSCs with qualities including, but not limited to, high clonality, sustainable self-renewal, minimal spontaneous differentiation and genomic abnormality, and high survivability as dissociated single cells. Methods and compositions, and particularly the novel culture media and plasmid vector systems, of embodiments according to the present disclosure address this need and provide other related advantages in the field of cellular reprogramming.

By using an efficient but transient and temporal expression system that minimizes the presence of exogenous genes for reduced probability of host genome integration, it is an object of the present disclosure to provide methods and compositions efficient in generating an iPSC without comprising exogenous DNA introduced to a non-pluripotent cell for induction of reprogramming. It is an object of the present disclosure to provide a combinational plasmid system to efficiently produce iPSCs with a “naïve” or “grounded” state of pluripotency and/or high clonality. iPSCs having ground state pluripotency enable long term survival and genetic stability of single cell dissociated iPSCs, and thus make it possible to generate clonal iPSC lines suitable for banking and manipulation such as single cell sorting and/or depletion, clonal iPSC targeted genomic editing, and directed redifferentiation of a homogenous population of iPSCs. Therefore, it is also an object of the present disclosure to provide methods and compositions to generate single cell derived iPSC clonal lines, or derivative cells therefrom, comprising one or several genetic modifications at selected sites, which include polynucleotides insertion, deletion, and substitution, and which modifications are retained and remain functional in subsequently derived cells after differentiation, dedifferentiation, reprogramming, expansion, passaging and/or transplantation.

One aspect of the present application provides a composition (e.g., FMM2) for induced pluripotent stem cell (iPSC) production, the composition comprising (i) a TGFβ family protein, (ii) a ROCK inhibitor, and (iii) a MEK inhibitor and a WNT activator, wherein the composition does not comprise a TGFβ inhibitor, wherein the composition is effective to improve iPSC pluripotency and genomic stability in a long-term iPSC maintenance. In some embodiments, the long-term iPSC maintenance comprises one or more of stages comprising: single cell dissociation of iPSC colonies, single cell sorting of dissociated iPSCs, iPSC single cell clonal expansion, clonal iPSC master cell bank (MCB) cryopreservation, thawing of iPSC MCB, and optionally additional cryopreserve-thaw cycles of the iPSC MCB; or the TGFβ family protein is optionally added to the composition at single cell dissociation of iPSC colonies, or at iPSC single cell clonal expansion, or at any stage in-between; or the MEK inhibitor and/or the WNT activator is at an amount 30-60% of that is used in a reprogramming composition for reprogramming a non-pluripotent cell to the iPSC. In some embodiments, the TGFβ family protein comprises at least one of Activin A, TGFβ, Nodal, and functional variants or fragments thereof, and/or the WNT activator comprises a GSK3 inhibitor. In some embodiments, the non-pluripotent cell comprises a somatic cell, a progenitor cell, or a multipotent cell; or the non-pluripotent cell comprises a T cell; or the reprogramming composition comprises a ROCK inhibitor, a MEK inhibitor, a WNT activator, a TGFβ inhibitor, and optionally an HDAC inhibitor, wherein the TGFβ inhibitor and the HDAC inhibitor are included in the reprogramming composition at specific stages during reprogramming.

In some embodiments, the improved long-term iPSC pluripotency is indicated by reduced pluripotency reversion or reduced spontaneous differentiation as compared to iPSCs without contact of the composition; and the improved genomic stability is indicated by a lower propensity for genomic abnormalities as compared to iPSCs without contact of the composition. In some embodiments, the improved genomic stability comprises reduction or prevention of trisomy or karyotype abnormality in iPSCs obtained from reprogramming a T cell. In some embodiments, the composition further comprises an iPSC, optionally wherein the iPSC comprises at least one genomic edit.

In some embodiments, the iPSC maintenance further comprises iPSC genetic editing to obtain an engineered iPSC pool, single sell sorting of engineered iPSC pool, engineered iPSC single cell clonal expansion, clonal engineered iPSC master cell bank (MCB) cryopreservation, thawing of engineered iPSC MCB, and optionally additional cryopreserve-thaw cycles of the engineered iPSC MCB; and the engineered iPSC comprises at least one genomic edit.

In another aspect, the invention provides a composition (e.g., FRM2) for induced pluripotent stem cell (iPSC) production, the composition comprising (i) a ROCK inhibitor, a MEK inhibitor, and a WNT activator; (ii) an HDAC inhibitor; and (iii) a TGFβ inhibitor, wherein the composition is effective to improve reprogramming of a non-pluripotent cell to obtain iPSCs having established pluripotency and improved genomic stability, and optionally, wherein, addition of (i), (ii) or (iii) to the composition is stage-specific during reprogramming of the non-pluripotent cell for an increased reprogramming efficiency. In some embodiments, the reprogramming of the non-pluripotent cell comprises one or more stages comprising: somatic cell transfection (day 0), exogenous gene expression, increase of heterochromatin, loss of somatic cell identity, and iPSC colony formation; or the addition of the HDAC inhibitor is optionally at chromatin restructuring, or at around day 2-3 (post transfection); or the addition of the TGFβ inhibitor is optionally at the loss of somatic cell identity, or at around day 6-8 (post transfection), wherein the one or more stages in reprogramming is indicated by cell morphological change and/or marker gene profiling.

In some embodiments, the HDAC inhibitor comprises valproic acid (VPA) or a functional variant or derivative thereof; and/or the WNT activator comprises a GSK3 inhibitor. In some embodiments, the non-pluripotent cell comprises a somatic cell, a progenitor cell, or a multipotent cell; or the non-pluripotent cell comprises a T cell. In some embodiments, the established pluripotency comprises a ground state pluripotency; and/or the established pluripotency is represented by increased naïve-specific gene expression comprising one or more of: MAEL, KLF4, DNMT3L, DPPA5, PRDM14, FGF4, UTF1, TFCP2L1, and TBX3; and/or wherein the improved genomic stability is indicated by a lower propensity for genomic abnormalities than iPSCs obtained without contact of the composition during reprogramming; and/or the increased reprogramming efficiency is indicated by the higher percentage of cells expressing pluripotency maker genes in an iPSC pool after reprogramming than that of the iPSC pool obtained without contact of the composition during reprogramming.

In yet another aspect, the invention provides a method of producing induced pluripotent stem cell (iPSC), comprising a step of cryopreserving a population of iPSCs, wherein the iPSCs are in contact with the composition as described herein (e.g., FRM2), and wherein pluripotency and genomic stability of the iPSCs are maintained during cryopreservation; and optionally, wherein the population of iPSCs comprising homogeneous iPSCs is expanded from a clonal iPSC single cell.

In some embodiments, the method of producing induced pluripotent stem cell (iPSC) further comprises a step of expanding a single cell iPSC clone to obtain the population of clonal iPSCs, wherein the iPSCs are in contact with the composition described herein (e.g., FMM2), and wherein pluripotency and genomic stability of the iPSCs are maintained during expansion.

In some embodiments, the method of producing induced pluripotent stem cell (iPSC) further comprises a step of single cell sorting of dissociated iPSCs to obtain a single cell iPSC clone, wherein the iPSCs are in contact with the composition as described herein (e.g., FMM2), and wherein pluripotency and genomic stability of the iPSCs are maintained during single cell sorting.

In some embodiments, the method of producing induced pluripotent stem cell (iPSC) further comprises a step of dissociating iPSC colonies to single cell iPSCs, wherein the iPSCs are in contact with the composition described herein (e.g., FMM2), and wherein pluripotency and genomic stability of the iPSCs are maintained during iPSC single cell dissociation.

In some embodiments, the method of producing induced pluripotent stem cell (iPSC) further comprises a step of obtaining at least one colony comprising iPSCs generated from reprogramming a non-pluripotent cell.

In various embodiments of the method of producing induced pluripotent stem cell, the iPSCs are reprogrammed from a somatic cell, a progenitor cell, or a multipotent cell, or wherein the iPSCs are reprogrammed from a T cell. In various embodiments, the pluripotency comprises a ground state pluripotency; and/or the pluripotency is represented by increased naïve-specific gene expression comprising one or more of: MAEL, KLF4, DNMT3L, DPPA5, PRDM14, FGF4, UTF1, TFCP2L1, and TBX3; and/or the genomic stability comprises a lower propensity for genomic abnormalities than iPSCs in said step without contact of the composition described herein.

In yet another aspect, the invention provides a method of producing induced pluripotent stem cell (iPSC), wherein the method comprises (i) transferring to a non-pluripotent cell one or more reprogramming factors to initiate reprogramming of the cell; and (ii) contacting the cell after step (i) with the composition described herein (e.g., FRM2) for a sufficient period of time, thereby generating at least one colony comprising iPSCs by reprogramming the non-pluripotent cell. In some embodiments, the step of transferring comprises introducing to the non-pluripotent cell: (i) one or more first plasmids, wherein each of the first plasmids comprises a replication origin, and a polynucleotide encoding one or more reprogramming factors, but does not comprise polynucleotides encoding an EBNA or a variant thereof, wherein the one or more first plasmids collectively comprise polynucleotides encoding at least OCT4, or at least OCT4, YAP1, SOX2 and large T antigen (LTag); wherein the introduction of one or more first plasmids induces a reprogramming process; and (ii) one of: (1) a second plasmid comprising a nucleotide sequence encoding an EBNA, wherein the second plasmid does not comprise a replication origin or polynucleotide(s) encoding reprogramming factor(s); (2) an EBNA mRNA; and (3) an EBNA protein. In some embodiments, the one or more first plasmids further collectively comprise polynucleotides encoding one or both of SOX2 and KLF, or one or more of MYC, LIN28, ESRRB and ZIC3.

In some embodiments, the step of contacting further comprises culturing the cells in presence of a ROCK inhibitor, a MEK inhibitor, a WNT activator, an HDAC inhibitor and a TGFβ inhibitor. In particular embodiments, the step of contacting comprises: (a) contacting the cell after step (i) with a combination comprising a ROCK inhibitor, a MEK inhibitor, and a WNT activator, optionally at a stage of exogenous reprogramming factor expression, or at day 1-2 post reprogramming factor transferring (day 0); (b) contacting the cell of step (a) with an HDAC inhibitor, optionally at a stage of chromatin restructuring, or at around day 2-3 post reprogramming factor transferring; and (c) contacting the cell of step (b) with a TGFβ inhibitor, optionally at a stage of loss of somatic cell identity, or at around day 6-8 (post transfection), thereby generating at least one colony comprising iPSCs; wherein said stage is indicated by cell morphological change and/or marker gene profiling; and/or wherein the iPSCs are footprint-free, have established pluripotency and improved genomic stability, and are produced with a higher efficiency as compared to reprogramming without steps (a), (b) and (c). In some embodiments, the non-pluripotent cell comprises a somatic cell, a progenitor cell, or a multipotent cell; or the non-pluripotent cell comprises a T cell. In some embodiments, the established pluripotency comprises a ground state pluripotency; and/or the established pluripotency is represented by increased naïve-specific gene expression comprising one or more of: MAEL, KLF4, DNMT3L, DPPA5, PRDM14, FGF4, UTF1, TFCP2L1, and TBX3; and/or the improved genomic stability comprises a lower propensity for genomic abnormalities than iPSCs from reprogramming without steps (a), (b) and (c); and/or the increased reprogramming efficiency is indicated by the higher percentage of cells expressing pluripotency marker genes in an iPSC pool after reprogramming than that of an iPSC pool obtained without contact of the composition during reprogramming. In some embodiments, the improved genomic stability further comprises reduction or prevention of trisomy or karyotype abnormality in iPSCs obtained from reprogramming a T cell.

In yet another aspect, the invention provides a method of producing induced pluripotent stem cell (iPSC), wherein the method comprises: (i) transferring to a non-pluripotent cell one or more reprogramming factors to initiate reprogramming of the cell; (ii) contacting the cell after step (i) with the composition described herein (e.g., FRM2) for a sufficient period of time, thereby generating at least one colony comprising iPSCs, wherein pluripotency and genomic stability of the iPSCs are established; (iii) dissociating the iPSC colony of step (ii) to dissociated iPSCs, wherein the iPSCs are in contact with the composition described herein (e.g., FMM2); (iv) sorting dissociated iPSCs to obtain one or more single cell iPSC clones, wherein the single cell iPSC clones are in contact with the composition described herein (e.g., FMM2); and optionally, (v) expanding the single cell iPSC clone to a population of clonal iPSCs, wherein the population of clonal iPSCs is in contact with the composition described herein (e.g., FMM2); and optionally (vi) cryopreserving the population of clonal iPSCs, wherein the cryopreserved population is in contact with the composition described herein (e.g., FMM2); wherein pluripotency and genomic stability of the iPSCs are maintained during the step of dissociating, sorting, expanding, cryopreserving, or thawing. In some embodiments, the one or more reprogramming factors comprise at least OCT4.

In various embodiments, the step (i) of transferring comprises introducing to the non-pluripotent cell: (a) one or more first plasmids, wherein each of the first plasmids comprises a replication origin, and a polynucleotide encoding one or more reprogramming factors, but does not comprise polynucleotides encoding an EBNA or a variant thereof, wherein the one or more first plasmids collectively comprise polynucleotides encoding at least OCT4, or at least OCT4, YAP1, SOX2 and large T antigen (LTag); wherein the introduction of one or more first plasmids induces a reprogramming process; and (b) one of: (1) a second plasmid comprising a nucleotide sequence encoding an EBNA, wherein the second plasmid does not comprise a replication origin or polynucleotide(s) encoding reprogramming factor(s); (2) an EBNA mRNA; and (3) an EBNA protein. In some embodiments, the one or more first plasmids further collectively comprise polynucleotides encoding one or both of SOX2 and KLF, or one or more of MYC, LIN28, ESRRB and ZIC3.

In some embodiments, the step (ii) of contacting further comprises culturing the cells in presence of a ROCK inhibitor, a MEK inhibitor, a WNT activator, an HDAC inhibitor and a TGFβ inhibitor. In particular embodiments, the step (ii) of contacting further comprises: (a) contacting the cell after step (i) with a combination comprising a ROCK inhibitor, a MEK inhibitor, and a WNT activator, optionally at a stage of exogenous reprogramming factor expression, or at day 1-2 post reprogramming factor transferring (day 0); (b) contacting the cell of step (a) with an HDAC inhibitor, optionally at a stage of chromatin restructuring, or at around day 2-3 post reprogramming factor transferring; and (c) contacting the cell of step (b) with a TGFβ inhibitor, optionally at a stage of loss of somatic cell identity, or at around day 6-8 (post transferring), thereby generating at least one colony comprising iPSCs; wherein said stage is indicated by cell morphological change and/or marker gene profiling; and/or wherein the iPSCs have established pluripotency and improved genomic stability, and are produced with a higher efficiency as compared to reprogramming without steps (a), (b) and (c). In some embodiments, the method further comprises: (1) contacting the cell of the sorting step (iv), expanding step (v), and cryopreserving step (vi), and optionally of the dissociating step (iii) with a ROCK inhibitor, a MEK inhibitor, and a WNT activator, wherein concentration of one or both of the MEK inhibitor and the WNT activator is 30%-60% of that in step (ii); and (2) additionally contacting the cell of the expanding step (v) and cryopreserving step (vi), and optionally of the dissociating step (iii) and/or sorting step (iv) with a TGFβ family protein; and wherein the cells in steps (iii), (iv), (v) and (vi) are not in contact with either a TGFβ inhibitor or an HDAC inhibitor.

In various embodiments, the non-pluripotent cell comprises a somatic cell, a progenitor cell, or a multipotent cell; or the non-pluripotent cell comprises a T cell. In some embodiments, the pluripotency comprises a ground state pluripotency; and/or the pluripotency is represented by increased naïve-specific gene expression comprising one or more of: MAEL, KLF4, DNMT3L, DPPA5, PRDM14, FGF4, UTF1, TFCP2L1, and TBX3; and/or the iPSC comprises at least one genomic edit; and/or the genomic stability comprises a lower propensity for genomic abnormalities. In some embodiments, the genomic stability further comprises reduction or prevention of trisomy or karyotype abnormality in iPSCs obtained from reprogramming a T cell.

In some embodiments, the method further comprises genetic editing of an iPSC to obtain an engineered iPSC pool, single sell sorting of engineered iPSC pool, engineered iPSC single cell clonal expansion, clonal engineered iPSC master cell bank (MCB) cryopreservation, thawing of engineered iPSC MCB, and optionally additional cryopreserve-thaw cycles of the engineered iPSC MCB; and wherein the engineered iPSC comprises at least one genomic edit. In some embodiments, the genomic edit leads to deletion or reduced expression of B2M, TAP1, TAP2, Tapasin, NLRC5, PD1, LAG3, TIM3, RFXANK, CITTA, RFX5, or RFXAP; or introduced or increased expression of HLA-E, HLA-G, CD16, 4-1BBL, CD3, CD4, CD8, CD47, CD137, CD80, PDL1, A2AR, CAR, TCR, engagers, or surface triggering receptors for engagers, in iPSC or iPSC-derived effector cells.

In yet another aspect, the invention provides a composition comprising an induced pluripotent cell (iPSC), a cell line, a clonal population or a master cell bank thereof, wherein the iPSC is contacted by a combination of a ROCK inhibitor, a MEK inhibitor, a WNT activator, and a TGFβ family protein, and wherein the iPSC comprises increased naïve-specific gene expression comprising one or more of: MAEL, KLF4, DNMT3L, DPPA5, PRDM14, FGF4, UTF1, TFCP2L1, and TBX3; and optionally the iPSC has at least one of the properties: high clonality, genetic stability, and ground state pluripotency. In some embodiments, the TGFβ family protein comprises at least one of Activin A, TGFβ, Nodal, and functional variants or fragments thereof; and/or wherein the WNT activator comprises a GSK3 inhibitor. In some embodiments, the iPSC is generated from reprogramming a non-pluripotent cell. In some embodiments, the non-pluripotent cell comprises a somatic cell, a progenitor cell, or a multipotent cell; or wherein the non-pluripotent cell comprises a T cell. In some embodiments, the iPSC comprises at least a genomic edit. In some embodiments, the composition further comprises a medium, wherein the medium is feeder-free.

In yet another aspect, the invention provides an induced pluripotent cell (iPSC), a cell line, a clonal population or a master cell bank thereof produced by any of the methods described herein. In some embodiments, the iPSC comprises at least a genomic edit.

In yet another aspect, the invention provides a derived non-natural cell or population thereof obtained from in vitro differentiation of the pluripotent cell or cell line described herein. In some embodiments, the cell is an immune effector cell, and optionally, the immune effector cell comprises at least a genomic edit comprised in the iPSC. In some embodiments, the cell comprises a CD34 cell, a hemogenic endothelium cell, a hematopoietic stem or progenitor cell, a hematopoietic multipotent progenitor cell, a T cell progenitor, an NK cell progenitor, a T cell, a NKT cell, an NK cell, a B cell, or an immune regulatory cell. In some embodiments, the cell is a rejuvenated cell comprising at least one of the following properties: global increase of heterochromatin; improved mitochondrial function; increased DNA damage responses; telomere elongation and decrease of percentage of short telomere; decrease in the fraction of senescent cells; and higher potential for proliferation, survival, persistence, or memory like functions, in comparison to its natural cell counterpart.

In yet another aspect, the invention provides a composition for use in manufacturing a pluripotent cell for application in cell-based therapies, wherein the composition comprises a pluripotent cell produced by the methods described herein. In some embodiments, the pluripotent cell is allogeneic or autologous. In yet another aspect, the invention provides a kit for medicament use comprising a pluripotent cell obtained by a method described herein. In yet another aspect, the invention provides a kit for medicament use comprising the induced pluripotent cell as described herein or the derived non-natural cell described herein.

Still another aspect of the present application provides an in vitro system for initiating reprogramming in a non-pluripotent cell, wherein the system comprises: one or more first plasmids, wherein each of the first plasmids comprises a replication origin, and a polynucleotide encoding one or more reprogramming factors but does not encode an EBNA or a derivative thereof; wherein the one or more first plasmids collectively comprise polynucleotides encoding OCT4, YAP1, SOX2 and LTag; and optionally one of (1) a second plasmid comprising a nucleotide sequence encoding an EBNA, wherein the second plasmid does not comprise a replication origin or polynucleotide(s) encoding reprogramming factor(s), (2) an EBNA mRNA, and (3) an EBNA protein. In some embodiments, the second plasmid of the system has a high rate of loss; and wherein the expression of EBNA by the second plasmid is short-lived, transient and temporal. In some other embodiments, the system does not provide EBNA replication and/or continuous expression in the nucleus. In one embodiment, the system could enable a transient/cytoplasmic expression of EBNA for a short duration, and prior to the appearance of pluripotency cell morphology and the induced expression of endogenous pluripotency genes. In some embodiments, the system enables a transient/cytoplasmic expression of one or more reprogramming factors comprised in the first plasmid(s) for a short duration, and prior to the appearance of pluripotency cell morphology and the induced expression of endogenous pluripotency genes.

In one embodiment of the system, the replication origin of first plasmid(s) is one selected from the group consisting of a Polyomavirinae virus, a Papillomavirinae virus, and a Gammaherpesvirinae virus. In some embodiments, the replication origin is one selected from the group consisting of SV40, BK virus (BKV), bovine papilloma virus (BPV), or Epstein-Barr virus (EBV). In one particular embodiment, the replication origin corresponds to, or is derived from, the wild-type replication origin of EBV. In some other embodiments, the EBNA of the second plasmid in the system is EBV-based. In some embodiments, the system provides one or more first plasmids collectively comprise polynucleotides encoding reprogramming factor(s) comprising (i) one or more of NANOG, KLF, LIN28, MYC, ECAT1, UTF1, ESRRB, HESRG, CDH1, TDGF1, DPPA4, DNMT3B, ZIC3, and L1TD1; or (ii) MYC, LIN28, ESRRB, and ZIC3. In some embodiments, the polynucleotides encoding reprogramming factors are comprised in a polycistronic construct or non-polycistronic construct in a first plasmid. In one embodiment of a polycistronic construct, it comprises a single open reading frame or multiple open reading frames. In the embodiment where the system comprises two or more first plasmids, each first plasmid may comprise the same or different reprogramming factors encoded by at least one copy of polynucleotide. In some embodiments, the system comprises four first plasmids, with each first plasmid comprising the same or different reprogramming factors encoded by at least one copy of the polynucleotide. In the embodiment where the system comprises four first plasmids, each first plasmid comprises at least one copy of polynucleotides encoding OCT4 and YAP1, SOX2 and MYC, LIN28 and LTag, and ESRRB and ZIC3, respectively.

In some embodiments of the system, the first plasmid comprises more than one polynucleotide encoding reprogramming factors, wherein the adjacent polynucleotides are operatively connected by a linker sequence encoding a self-cleaving peptide or an IRES. In one embodiment, the self-cleaving peptide is a 2A peptide is selected from the group comprising F2A, E2A, P2A and T2A. In another embodiment, the 2A peptides comprised in a first plasmid construct may be the same or different. In yet another embodiment where the plasmid of the system comprises multiple 2As, the two 2A peptides in neighboring positions are different. In some other embodiments of the system, the first and the second plasmid each comprises one or more promoters for expression of reprogramming factors and EBNA, and the one or more promoters comprise at least one of CMV, EF1α, PGK, CAG, UBC, and other suitable promoters that are constitutive, inducible, endogenously regulated, or temporal-, tissue- or cell type-specific. In one embodiment, the first and the second plasmid each comprises a CAG promoter.

In one aspect, the present disclosure provides kits. In some embodiments, the kit comprises one or more compositions disclosed herein, such as (i) a first composition for iPSC production and a second composition for iPSC production, and/or (ii) a first composition for iPSC production and a second composition for iPSC maintenance. Also provided is a kit comprising the in vitro system as described herein.

Various objects and advantages of the use of the present methods and compositions will become apparent from the following description taken in conjunction with the accompanying drawings wherein are set forth, by way of illustration and example, certain embodiments of this invention.

### BRIEF DESCRIPTION OF THE DRAWINGS

FIGS. 1A and 1B show that supplementing FMM with members of the TGFβ family (such as Activin A, TGFβ and/or Nodal) and/or concentration reduction in MEK and GSK3 inhibitors in FMM enhance long-term stability of iPSCs. FIG. 1A. The diagram shows an exemplary experimental design for testing different formulations of FMM-based media for long-term iPSC culture. FIG. 1B. A summary of ddPCR and karyotype results from one engineered clone derived from T cell donor 1, and two non-engineered clones from T cell donor 2 tested using the experimental design of FIG. 1A.

FIG. 2 shows results from PCA analysis of three TiPSC clones maintained in E8, FMM or FMM2 for more than 10 passages.

FIGS. 3A and 3B show heatmaps of key pluripotent markers of the three TiPSC clones from FIG. 2, with common pluripotency markers expressed in all conditions tested (FIG. 3A) and expression of naïve-specific markers being further elevated by addition of Activin A (FIG. 3B).

FIGS. 4A-4C are schematic and graphical representations showing that FMM supplemented with Activin A prevents stress-induced genomic abnormalities. FIG. 4A shows an exemplary experimental design for inducing stress (engineering and/or screening/expansion) of iPSC clones. FIG. 4B shows a comparison of abnormal cells in the resulting iPSC population using FMM and FMM2 (FMM+ActA). FIG. 4C shows a comparison of abnormal clones post cryopreservation in the resulting iPSC population using FMM and FMM2 (FMM+ActA).

FIGS. 5A and 5B show that application of new FMM formulations earlier in the iPSC generation process leads to improved genomic stability of iPSCs. FIG. 5A is a diagram showing an exemplary experimental design for iPSC generation from primary T cells. FIG. 5B shows the results of genomic stability evaluations of iPSC clones generated with FMM compared to new FMM formulations.

FIG. 6 is a diagram showing exemplary DNA constructs of vector 1(1), vector 1(2) . . . vector 1(n), vector 1(n+1), and a vector 2 used in a Short-lived Transient and Temporal Reprogramming (STTR) system.

FIGS. 7A and 7B show that addition of valproic acid (VPA) to STTR system improves reprogramming efficiency of STTR. FIG. 7A shows flow cytometry analysis for expression of iPSC surface markers (SSEA4, TRA-1-81, and CD30) in T cells reprogrammed using a STTR2 system with or without VPA treatment. FIG. 7B shows that VPA treatment potentiates STTR2 reprogramming of T cells derived from three different donors.

FIGS. 8A-8C demonstrate STTR2-induced stable pluripotent cultures derived from T cells of multiple donors. FIG. 8A shows images of iPSC colonies induced by the STTR2 system from T cells of 2 different donors. FIG. 8B shows that fractions of iPSC populations increased over continuous passaging indicating stable pluripotent cultures derived from multiple donors. FIG. 8C shows representative flow cytometry profiles of reprogramming pools from T cells of two different donors.

FIG. 9 is a diagram showing an exemplary workflow for improved reprogramming and iPSC maintenance using stage-specific media.

FIGS. 10A-10C show that reprogramming T cells using an STTR2 system led to robust generation of iPSC clones that are transgene-free. FIG. 10A shows an illustration of the locations of the TaqMan probes (black bars) for detection of reprogramming vectors. FIG. 10B shows examples of mean Ct values from TaqMan assays; Positive control 1 is an iPSC clone with multiple transgene integrations (positive for EBNA1 and P2A); Positive control 2 has integration of plasmid backbone (positive for KanR); ND: Not detected. FIG. 10C shows a summary of vector clearance results of STTR2 clones derived from T cells of 2 different donors.

FIG. 11 demonstrates flow cytometry profiles of STTR2-generated iPSCs clones showing homogenous expression of iPSC surface markers (SSEA4, TRA-1-81 and CD30).

FIGS. 12A-12C demonstrate that STTR2-generated iPSC clones maintain high propensity to differentiate into cell types representing all three germ layers. FIG. 12A shows expression of indicated lineage markers (pancreatic progenitor marker SOX17 for endoderm, mesenchymal marker CD56 for mesoderm and neural progenitor marker SOX2 for ectoderm).

FIG. 12B shows flow cytometry analyses at indicated timepoints demonstrating that STTR2-generated iPSCs differentiated into mature T cells similar to control iPSCs generated using a conventional episomal system. FIG. 12C provides images of tissue sections from each of the three germ layers formed in STTR2-generated iPSC teratoma.

FIG. 13 demonstrates flow cytometry profiles of STTR2-generated, CAR-engineered iPSCs showing homogenous expression of pluripotency surface markers.

FIGS. 14A and 14B demonstrate phenotypical and functional properties of T cells derived from CAR-engineered STTR2-generated iPSCs.

### DETAILED DESCRIPTION OF THE INVENTION

### Definitions

Unless defined otherwise, all technical and scientific terms used herein have the same meaning as commonly understood by those of ordinary skill in the art to which the invention belongs. For the purposes of the present invention, the following terms are defined below. The articles “a,” “an,” and “the” are used herein to refer to one or to more than one (i.e., to at least one) of the grammatical object of the article. By way of example, “an element” means one element or more than one element.

The use of the alternative (e.g., “or”) should be understood to mean either one, both, or any combination thereof of the alternatives. The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or 1% about a reference quantity, level, value, number, frequency, percentage, dimension, size, amount, weight or length.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

As used herein, the terms “substantially free of” and “essentially free of” are used interchangeably, and when used to describe a composition, such as a cell population or culture media, refer to a composition that is free of a specified substance or its source thereof, such as, 95% free, 96% free, 97% free, 98% free, 99% free of the specified substance or its source thereof, or is undetectable as measured by conventional means. The term “free of” or “essentially free of” a certain ingredient or substance in a composition also means that no such ingredient or substance is (1) included in the composition at any concentration, or (2) included in the composition functionally inert, but at a low concentration. Similar meaning can be applied to the term “absence of,” where referring to the absence of a particular substance or its source thereof of a composition.

As used herein, the term “isolated” or the like refers to a cell, or a population of cells, which has been separated from its original environment, i.e., the environment of the isolated cells is substantially free of at least one component as found in the environment in which the “un-isolated” reference cells exist. The term includes a cell that is removed from some or all components as it is found in its natural environment, for example, tissue, biopsy. The term also includes a cell that is removed from at least one, some or all components as the cell is found in non-naturally occurring environments, for example, culture, cell suspension. Therefore, an isolated cell is partly or completely separated from at least one component, including other substances, cells or cell populations, as it is found in nature or as it is grown, stored or subsisted in non-naturally occurring environments. Specific examples of isolated cells include partially pure cells, substantially pure cells and cells cultured in a medium that is non-naturally occurring. Isolated cells may be obtained from separating the desired cells, or populations thereof, from other substances or cells in the environment, or from removing one or more other cell populations or subpopulations from the environment.

As used herein, the term “purify” or the like refers to increasing purity. For example, the purity can be increased to at least 50%, 60%, 70%, 80%, 90%, 95%, 99%, or 100%.

Throughout this specification, unless the context requires otherwise, the words “comprise,” “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured in a laboratory apparatus, usually under sterile conditions, and typically for a few hours or up to about 24 hours, but including up to 48 or 72 hours or longer, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The term “in vivo” refers generally to activities that take place inside an organism.

As used herein, the terms “reprogramming” or “dedifferentiation” or “increasing cell potency” or “increasing developmental potency” refers to a method or a process of increasing the pluripotency of a cell or dedifferentiating the cell to a less differentiated state. For example, a cell that has an increased cell pluripotency has more developmental plasticity (i.e., can differentiate into more cell types) compared to the same cell in the non-reprogrammed state. In other words, a reprogrammed cell is one that is in a less differentiated state than the same cell in a non-reprogrammed state. A “reprogramming cell,” as opposed to a reprogrammed cell, refers to a non-pluripotent cell undergoing reprogramming/dedifferentiation to a pluripotent state, presenting a transitional morphology (i.e., a change in morphology) yet without the hallmarks of a pluripotent cell, including pluripotent stem cell morphology or stable endogenous pluripotency gene expression such as OCT4, NANOG, SOX2, SSEA4, TRA181, CD30 and/or CD50. The transitional morphology of a “reprogramming cell” distinguishes the cell from the starting non-pluripotent cell prior to reprogramming induction, as well as from a reprogrammed cell having the embryonic stem cell hallmark morphology. For example, when reprogramming a fibroblast, the morphological change of the reprogramming cell comprises MET (mesenchymal to epithelial transition). A person skilled in the art understands and identifies readily such transitional morphology for various types of somatic cell induced to reprogram. In some embodiments, the reprogramming cells are intermediary cells that have been induced to reprogram for at least 1, 2, 3, 4, 5, 6, 7, 8, or more days, but no more than 21, 22, 24, 26, 28, 30, 32, 35, 40 days or any number of days in between, wherein the cells have not entered a self-maintaining or self-sustaining pluripotent state. A non-pluripotent cell is induced to reprogram when the cell is introduced with one or more reprogramming factors. A reprogramming cell that has been induced to reprogram for 1, 2, 3, or 4 days is a cell 1, 2, 3, or 4 days post transduction of the reprogramming factors (the day of transduction is day 0). Unlike the somatic cell prior to the exposure to the exogenous expression of reprogramming factors, a reprogramming cell can progress within the reprogramming process to reach a stable pluripotent state and becomes a reprogrammed cell even without the presence of the exogenous expression reprogramming factors, so long as a sufficient time period is given.

As used herein, the term “induced pluripotent stem cells” or “iPSCs” means that the stem cells are produced from differentiated adult, neonatal or fetal cells that have been induced or changed (i.e., reprogrammed) into cells capable of differentiating into tissues of all three germ or dermal layers: mesoderm, endoderm, and ectoderm.

As used herein, the term “embryonic stem cell” refers to naturally occurring pluripotent stem cells of the inner cell mass of the embryonic blastocyst. Embryonic stem cells are pluripotent and give rise during development to all derivatives of the three primary germ layers: ectoderm, endoderm and mesoderm. They do not contribute to the extra-embryonic membranes or the placenta and are not totipotent.

As used herein, the term “multipotent stem cell” refers to a cell that has the developmental potential to differentiate into cells of one or more germ layers (ectoderm, mesoderm and endoderm), but not all three. Thus, a multipotent cell can also be termed a “partially differentiated cell.” Multipotent cells are well known in the art, and examples of multipotent cells include adult stem cells, such as for example, hematopoietic stem cells and neural stem cells. “Multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, the term “multipotency” refers to a state of a cell with a degree of developmental potential that is less than totipotent and pluripotent.

As used herein, the term “pluripotent” refers to the ability of a cell to form all lineages of the body or soma (i.e., the embryo proper). For example, embryonic stem cells are a type of pluripotent stem cells that are able to form cells from each of the three germ layers: the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

Pluripotency can be determined, in part, by assessing pluripotency characteristics of the cells. Pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60, TRA1-81, TRA1-85, TRA2-54, GCTM-2, TG343, TG30, CD9, CD29, CD133/prominin, CD140a, CD56, CD73, CD90, CD105, OCT4, NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

Two types of pluripotency have previously been described: the “primed” or “metastable” state of pluripotency akin to the epiblast stem cells (EpiSC) of the late blastocyst, and the “Naïve” or “Ground” state of pluripotency akin to the inner cell mass of the early/preimplantation blastocyst. While both pluripotent states exhibit the characteristics as described above, the naïve or ground state further exhibits: (i) pre-inactivation or reactivation of the X-chromosome in female cells; (ii) improved clonality and survival during single-cell culturing; (iii) global reduction in DNA methylation; (iv) reduction of H3K27me3 repressive chromatin mark deposition on developmental regulatory gene promoters; and (v) reduced expression of differentiation markers relative to primed state pluripotent cells. Standard methodologies of cellular reprogramming in which exogenous pluripotency genes are introduced to a somatic cell, expressed, and then either silenced or removed from the resulting pluripotent cells are generally seen to have characteristics of the primed-state of pluripotency. Under standard pluripotent cell culture conditions such cells remain in the primed state unless the exogenous transgene expression is maintained, wherein characteristics of the ground-state are observed.

Pluripotency exists as a continuum and induced pluripotent stem cells appear to exist in both a “primed” state and a “naïve” state, with a cell in a naïve state possibly having greater differentiation potential. Induced pluripotent stem cells generated in conventional culture medium exist in a primed state and more closely resemble cells derived from a post-implantation blastocyst, while naïve iPSCs display pluripotency characteristics that more closely resemble mouse embryonic stem cells or cells derived from a pre-implantation blastocyst. The primed and naïve cell states can be defined by various differences, including differences in colony morphology, cellular response to inhibition or activation of key signaling pathways, gene expression signature, and ability to reactivate genes associated with extraembryonic cells. For example, conventional iPSCs, representing a primed pluripotent state, exhibit a colony morphology that is flat, while naïve iPSCs exhibit a compact domed colony morphology that is similar to mouse embryonic stem cells. As used herein, the term “pluripotent stem cell morphology” refers to the classical morphological features of an embryonic stem cell. Normal embryonic stem cell morphology is characterized by being round and compact in shape, with a high nucleus-to-cytoplasm ratio, the notable presence of nucleoli, and typical inter-cell spacing.

A “pluripotency factor” or “reprogramming factor” refers to an agent or a combination of agents used for inducing or increasing the developmental potency of a cell. Pluripotency factors include, without limitation, polynucleotides, polypeptides, and small molecules capable of increasing the developmental potency of a cell. Exemplary pluripotency factors include, for example, transcription factors OCT4 and SOX2, and small molecule reprogramming agents, for example, TGFβ inhibitor, GSK3 inhibitor, MEK inhibitor and ROCK inhibitor.

As used herein, the term “differentiation” is the process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

Differentiation of pluripotent stem cells requires a change in the culture system, such as changing the stimuli agents in the culture medium or the physical state of the cells. The most conventional strategy utilizes the formation of embryoid bodies (EBs) as a common and critical intermediate to initiate the lineage-specific differentiation. “Embryoid bodies” are three-dimensional clusters that have been shown to mimic embryo development as they give rise to numerous lineages within their three-dimensional area. Through the differentiation process, typically a few hours to days, simple EBs (for example, aggregated pluripotent stem cells elicited to differentiate) continue maturation and develop into a cystic EB at which time, typically days to a few weeks, they are further processed to continue differentiation. EB formation is initiated by bringing pluripotent stem cells into close proximity with one another in three-dimensional multilayered clusters of cells, typically this is achieved by one of several methods including allowing pluripotent cells to sediment in liquid droplets, sedimenting cells into “U” bottomed well-plates or by mechanical agitation. To promote EB development, the pluripotent stem cell aggregates require further differentiation cues, as aggregates maintained in pluripotent culture maintenance medium do not form proper EBs. As such, the pluripotent stem cell aggregates need to be transferred to differentiation medium that provides eliciting cues towards the lineage of choice. EB-based culture of pluripotent stem cells typically results in generation of differentiated cell populations (ectoderm, mesoderm and endoderm germ layers) with modest proliferation within the EB cell cluster. Although proven to facilitate cell differentiation, EBs, however, give rise to heterogeneous cells in variable differentiation states because of the inconsistent exposure of the cells in the three-dimensional structure to differentiation cues from the environment. In addition, EBs are laborious to create and maintain. Moreover, cell differentiation through EB is accompanied by modest cell expansion, which also contributes to low differentiation efficiency.

In comparison, “aggregate formation,” as distinct from “EB formation,” can be used to expand the populations of pluripotent stem cell derived cells. For example, during aggregate-based pluripotent stem cell expansion, culture media are selected to maintain proliferation and pluripotency. Cell proliferation generally increases the size of the aggregates forming larger aggregates, these aggregates can be routinely mechanically or enzymatically dissociated into smaller aggregates to maintain cell proliferation within the culture and increase numbers of cells. As distinct from EB culture, cells cultured within aggregates in maintenance culture maintain markers of pluripotency. The pluripotent stem cell aggregates require further differentiation cues to induce differentiation.

As used herein, “monolayer differentiation” is a term referring to a differentiation method distinct from differentiation through three-dimensional multilayered clusters of cells, i.e., “embryoid bodies”, “EBs”, or “EB formation.” Monolayer differentiation, among other advantages disclosed herein, avoids the need for EB formation for initiating differentiation. Because monolayer culturing does not mimic embryo development, such as EB formation, monolayer differentiation is directed towards specific lineages as desired, as compared to all three germ layer differentiation in EB.

As used herein, a “dissociated” cell refers to a cell that has been substantially separated or purified away from other cells or from a surface (e.g., a culture plate surface). For example, cells can be dissociated from an animal or tissue by mechanical or enzymatic methods. Alternatively, cells that aggregate in vitro can be dissociated from each other, such as by dissociation into a suspension of clusters, single cells or a mixture of single cells and clusters, enzymatically or mechanically. In yet another alternative embodiment, adherent cells are dissociated from a culture plate or other surface. Dissociation thus can involve breaking cell interactions with extracellular matrix (ECM) and substrates (e.g., culture surfaces), or breaking the ECM between cells.

As used herein, “feeder cells” or “feeders” are terms describing cells of one type that are co-cultured with cells of a second type to provide an environment in which the cells of the second type can grow, as the feeder cells provide growth factors and nutrients for the support of the second cell type. The feeder cells are optionally from a different species as the cells they are supporting. For example, certain types of human cells, including stem cells, can be supported by primary cultures of mouse embryonic fibroblasts, or immortalized mouse embryonic fibroblasts. The feeder cells may typically be inactivated when being co-cultured with other cells by irradiation or treatment with an anti-mitotic agent such as mitomycin to prevent them from outgrowing the cells they are supporting. Feeder cells may include endothelial cells, stromal cells (for example, epithelial cells or fibroblasts), and leukemic cells. Without limiting the foregoing, one specific feeder cell type may be a human feeder, such as a human skin fibroblast. Another feeder cell type may be mouse embryonic fibroblasts (MEF). In general, various feeder cells can be used in part to maintain pluripotency, direct differentiation towards a certain lineage and promote maturation to a specialized cell types, such as an effector cell.

As used herein, a “feeder-free” (FF) environment refers to an environment such as a culture condition, cell culture or culture media which is essentially free of feeder cells, and/or which has not been pre-conditioned by the cultivation of feeder cells. “Pre-conditioned” medium refers to a medium harvested after feeder cells have been cultivated within the medium for a period of time, such as for at least one day. Pre-conditioned medium contains many mediator substances, including growth factors and cytokines secreted by the feeder cells. In some embodiments, a feeder-free environment is free of both feeder cells and is also not pre-conditioned by the cultivation of feeder cells. Feeder cells include, but without limitation, stromal cells, mouse embryonic fibroblasts, human fibroblasts, keratinocytes, and embryonic stem cells.

“Culture” or “cell culture” refers to the maintenance, growth and/or differentiation of cells in an in vitro environment. ““Cell culture media,” “culture media” (singular “medium” in each case), “supplement” and “media supplement” refer to nutritive compositions that cultivate cell cultures.

“Cultivate” or “maintain” refers to the sustaining, propagating (growing) and/or differentiating of cells outside of tissue or the body, for example in a sterile plastic (or coated plastic) cell culture dish or flask. “Cultivation” or “maintaining” may utilize a culture medium as a source of nutrients, hormones and/or other factors helpful to propagate and/or sustain the cells.

As used herein, “passage” or “passaging” refers to the act of splitting the cultured cells by subdividing and plating cells into multiple cell culture surfaces or vessels when the cells have proliferated to a desired extent. In some embodiments “passage” or “passaging” refers to subdividing, diluting and plating the cells. As cells are passaged from the primary culture surface or vessel into a subsequent set of surfaces or vessels, the subsequent cultures may be referred to herein as “secondary culture” or “first passage,” etc. Each act of subdividing and plating into a new culture vessel is considered one passage. In some embodiments, the cultured cells are passaged every 1, 2, 3, 4, 5, 6, 7, or more, days. In some embodiments, the initially selected iPSCs after reprogramming are passaged once every 3-7 days.

“Functional” as used in the context of genomic editing or modification of iPSC, and derived non-pluripotent cells differentiated therefrom, or genomic editing or modification of non-pluripotent cells and derived iPSCs reprogrammed therefrom, refers to (1) at the gene level-successful knocked-in, knocked-out, knocked-down gene expression, transgenic or controlled gene expression such as inducible or temporal expression at a desired cell development stage, which is achieved through direct genomic editing or modification, or through “passing-on” via differentiation from or reprogramming of a starting cell that is initially genomically engineered; or (2) at the cell level-successful removal, adding, or altering a cell function/characteristics via (i) gene expression modification obtained in said cell through direct genomic editing, (ii) gene expression modification maintained in said cell through “passing-on” via differentiation from or reprogramming of a starting cell that is initially genomically engineered; (iii) down-stream gene regulation in said cell as a result of gene expression modification that only appears in an earlier development stage of said cell, or only appears in the starting cell that gives rise to said cell via differentiation or reprogramming; or (iv) enhanced or newly attained cellular function or attribute displayed within the mature cellular product, initially derived from the genomic editing or modification conducted at the iPSC, progenitor or dedifferentiated cellular origin.

As used herein, the term “genetic imprint” refers to genetic or epigenetic information that contributes to preferential therapeutic attributes in a source cell or an iPSC, and is retainable in the source cell derived iPSCs, and/or the iPSC-derived non-natural hematopoietic lineage cells. As used herein, “a source cell” is a non-pluripotent cell that may be used for generating iPSCs through reprogramming, and the source cell derived iPSCs may be further differentiated to specific cell types including any hematopoietic lineage cells. The source cell derived iPSCs, and differentiated cells therefrom are sometimes collectively called “derived cells” depending on the context. As used herein, the genetic imprint(s) conferring a preferential therapeutic attribute is incorporated into the iPSCs either through reprogramming a selected source cell that is donor-, disease-, or treatment response-specific, or through introducing genetically modified modalities to iPSC using genomic editing. In the aspect of a source cell obtained from a specifically selected donor, disease or treatment context, the genetic imprint contributing to preferential therapeutic attributes may include any context specific genetic or epigenetic modifications which manifest a retainable phenotype, i.e., a preferential therapeutic attribute, that is passed on to derivative cells of the selected source cell, irrespective of the underlying molecular events being identified or not. Donor-, disease-, or treatment response-specific source cells may comprise genetic imprints that are retainable in iPSCs and derived hematopoietic lineage cells, which genetic imprints include but are not limited to, prearranged monospecific TCR, for example, from a viral specific T cell or invariant natural killer T (iNKT) cell; trackable and desirable genetic polymorphisms, for example, homozygous for a point mutation that encodes for the high-affinity CD16 receptor in selected donors; and predetermined HLA requirements, i.e., selected HLA-matched donor cells exhibiting a haplotype with increased population. As used herein, preferential therapeutic attributes include improved engraftment, trafficking, homing, viability, self-renewal, persistence, immune response regulation and modulation, survival, and cytotoxicity of a derived cell. A preferential therapeutic attribute may also relate to antigen targeting receptor expression; HLA presentation or lack thereof; resistance to tumor microenvironment; induction of bystander immune cells and immune modulations; improved on-target specificity with reduced off-tumor effect; resistance to treatment such as chemotherapy.

As used herein, “genetic modification” refers to genetic editing including those (1) naturally derived from rearrangements, mutations, genetic imprinting and/or epigenetic modification that take place in a cell or in cell development, or (2) obtained through genomic engineering through cell manipulation including, but not limited to, insertion, deletion or substitution in the genome of a cell. Genetic modification, as used herein, also includes one or more retainable therapeutic attributes of a source-specific immune cell that is donor-, disease-, or treatment response-specific. Genetically modified cells are cells comprising the genetic modification (e.g., a genetic edit) as compared to corresponding wildtype cells that do not have such genetic modification.

The term “enhanced therapeutic property” as used herein, refers to a therapeutic property of a cell that is enhanced as compared to a typical cell of the same general cell type. In the context of immune cells, for example, an NK cell with an “enhanced therapeutic property” will possess an enhanced, improved, and/or augmented therapeutic property as compared to a typical, unmodified, and/or naturally occurring NK cell. Therapeutic properties of an immune cell may include, but are not limited to, cell engraftment, trafficking, homing, viability, self-renewal, persistence, immune response regulation and modulation, survival, and cytotoxicity. Therapeutic properties of an immune cell are also manifested by antigen targeting receptor expression; HLA presentation or lack thereof; resistance to tumor microenvironment; induction of bystander immune cells and immune modulations; improved on-target specificity with reduced off-tumor effect; resistance to treatment such as chemotherapy.

By “integration” it is meant that one or more nucleotides of a construct is stably inserted into the cellular genome, i.e., covalently linked to the nucleic acid sequence within the cell's chromosomal DNA. By “targeted integration” it is meant that the nucleotide(s) of a construct is inserted into the cell's chromosomal or mitochondrial DNA at a pre-selected site or “integration site”. The term “integration” as used herein further refers to a process involving insertion of one or more exogenous sequences or nucleotides of the construct, with or without deletion of an endogenous sequence or nucleotide at the integration site. In the case where there is a deletion at the insertion site, “integration” may further comprise replacement of the endogenous sequence or a nucleotide that is deleted with the one or more inserted nucleotides.

A “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. The term “vector” as used herein comprises the construct to be delivered. A vector can be a linear or a circular molecule. A vector can be integrating or non-integrating. The major types of vectors include, but are not limited to, plasmids, episomal vector, viral vectors, cosmids, and artificial chromosomes. Viral vectors include, but are not limited to, adenovirus vector, adeno-associated virus vector, retrovirus vector, lentivirus vector, Sendai virus vector, and the like.

As used herein, the term “encoding” refers to the inherent property of specific sequences of nucleotides in a polynucleotide, such as a gene, a cDNA, or a mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “exogenous” in intended to mean that the referenced molecule or the referenced activity is introduced into the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell. Similarly, the term when used in reference to expression of an encoding nucleic acid refers to expression of an encoding nucleic acid contained within the cell and not exogenously introduced.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e., a polypeptide found in nature) or fragment thereof; a variant polypeptide (i.e., a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

As used herein, the term “polynucleotide” refers to a polymeric form of nucleotides of any length, either deoxyribonucleotides or ribonucleotides or analogs thereof. The sequence of a polynucleotide is composed of four nucleotide bases: adenine (A); cytosine (C); guanine (G); thymine (T); and uracil (U) for thymine when the polynucleotide is RNA. A polynucleotide can include a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. Polynucleotide also refers to both double- and single-stranded molecules.

As used herein, the term “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

“Operably linked” or “operatively linked,” as used herein, refers to the association of nucleic acid sequences on a single nucleic acid fragment so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory sequences in sense or antisense orientation.

As used herein, the term “engager” refers to a molecule, e.g., a fusion polypeptide, which is capable of forming a link between an immune cell (e.g., a T cell, a NK cell, a NKT cell, a B cell, a macrophage, or a neutrophil) and a tumor cell; and activating the immune cell. Examples of engagers include, but are not limited to, bi-specific T cell engagers (BiTEs), bi-specific killer cell engagers (BiKEs), tri-specific killer cell engagers, or multi-specific killer cell engagers, or universal engagers compatible with multiple immune cell types.

As used herein, the term “surface triggering receptor” refers to a receptor capable of triggering or initiating an immune response, e.g., a cytotoxic response. Surface triggering receptors may be engineered, and may be expressed on effector cells, e.g., a T cell, a NK cell, a NKT cell, a B cell, a macrophage, or a neutrophil. In some embodiments, the surface triggering receptor facilitates bi- or multi-specific antibody engagement between the effector cells and specific target cell e.g., a tumor cell, independent of the effector cell's natural receptors and cell types. Using this approach, one may generate iPSCs comprising a universal surface triggering receptor, and then differentiate such iPSCs into populations of various effector cell types that express the universal surface triggering receptor. By “universal”, it is meant that the surface triggering receptor can be expressed in, and activate, any effector cells irrespective of the cell type, and all effector cells expressing the universal receptor can be coupled or linked to the engagers having the same epitope recognizable by the surface triggering receptor, regardless of the engager's tumor binding specificities. In some embodiments, engagers having the same tumor targeting specificity are used to couple with the universal surface triggering receptor. In some embodiments, engagers having different tumor targeting specificity are used to couple with the universal surface triggering receptor. As such, one or multiple effector cell types can be engaged to kill one specific type of tumor cells in some case, and to kill two or more types of tumors in some other cases. A surface triggering receptor generally comprises a co-stimulatory domain for effector cell activation and an anti-epitope that is specific to the epitope of an engager. A bi-specific engager is specific to the anti-epitope of a surface triggering receptor on one end, and is specific to a tumor antigen on the other end.

As used herein, the term “safety switch protein” refers to an engineered protein designed to prevent potential toxicity or otherwise adverse effects of a cell therapy. In some instances, the safety switch protein expression is conditionally controlled to address safety concerns for transplanted engineered cells that have permanently incorporated the gene encoding the safety switch protein into its genome. This conditional regulation could be variable and might include control through a small molecule-mediated post-translational activation and tissue-specific and/or temporal transcriptional regulation. The safety switch could mediate induction of apoptosis, inhibition of protein synthesis, DNA replication, growth arrest, transcriptional and post-transcriptional genetic regulation and/or antibody-mediated depletion. In some instances, the safety switch protein is activated by an exogenous molecule, e.g., a prodrug, that when activated, triggers apoptosis and/or cell death of a therapeutic cell. Examples of safety switch proteins include, but are not limited to, suicide genes such as caspase 9 (or caspase 3 or 7), thymidine kinase, cytosine deaminase, B-cell CD20, modified EGFR, and any combination thereof. In this strategy, a prodrug that is administered in the event of an adverse event is activated by the suicide-gene product and kills the transduced cell.

As used herein, the term “pharmaceutically active proteins or peptides” refer to proteins or peptides that are capable of achieving a biological and/or pharmaceutical effect on an organism. A pharmaceutically active protein has healing curative or palliative properties against a disease and may be administered to ameliorate relieve, alleviate, reverse or lessen the severity of a disease. A pharmaceutically active protein also has prophylactic properties and is used to prevent the onset of a disease or to lessen the severity of such disease or pathological condition when it does emerge. Pharmaceutically active proteins include an entire protein or peptide or pharmaceutically active fragments thereof. It also includes pharmaceutically active analogs of the protein or peptide or analogs of fragments of the protein or peptide. The term pharmaceutically active protein also refers to a plurality of proteins or peptides that act cooperatively or synergistically to provide a therapeutic benefit. Examples of pharmaceutically active proteins or peptides include, but are not limited to, receptors, binding proteins, transcription and translation factors, tumor growth suppressing proteins, antibodies or fragments thereof, growth factors, and/or cytokines.

As used herein, the term “signaling molecule” refers to any molecule that modulates, participates in, inhibits, activates, reduces, or increases, the cellular signal transduction. Signal transduction refers to the transmission of a molecular signal in the form of chemical modification by recruitment of protein complexes along a pathway that ultimately triggers a biochemical event in the cell. Signal transduction pathways are well known in the art, and include, but are not limited to, G protein coupled receptor signaling, tyrosine kinase receptor signaling, integrin signaling, toll gate signaling, ligand-gated ion channel signaling, ERK/MAPK signaling pathway, Wnt signaling pathway, cAMP-dependent pathway, and IP3/DAG signaling pathway.

As used herein, the term “targeting modality” refers to a molecule, e.g., a polypeptide, that is genetically incorporated into a cell to promote antigen and/or epitope specificity that includes, but is not limited to, i) antigen specificity as it relates to a unique chimeric antigen receptor (CAR) or T cell receptor (TCR), ii) engager specificity as it relates to monoclonal antibodies or bispecific engager, iii) targeting of transformed cells, iv) targeting of cancer stem cells, and v) other targeting strategies in the absence of a specific antigen or surface molecule.

As used herein, the term “specific” or “specificity” can be used to refer to the ability of a molecule, e.g., a receptor or an engager, to selectively bind to a target molecule, in contrast to non-specific or non-selective binding.

“HLA deficient”, including HLA class I deficient, or HLA class II deficient, or both, refers to cells that either lack, or no longer maintain, or have reduced levels of surface expression of a complete MHC complex comprising an HLA class I protein heterodimer and/or an HLA class II heterodimer, such that the diminished or reduced level is less than the level naturally detectable by other cells or by synthetic methods. HLA class I deficiency can be achieved by functional deletion of any region of the HLA class I locus (chromosome 6p21), or deletion or reducing the expression level of HLA class I associated genes including, not being limited to, beta-2 microglobulin (B2M) gene, TAP 1 gene, TAP 2 gene and Tapasin. HLA class II deficiency can be achieved by functional deletion or reduction of HLA-II associated genes including, not being limited to, RFXANK, CIITA, RFX5 and RFXAP. It was previously unclear whether HLA complex deficient or altered iPSCs have the capacity to enter development, mature and generate functional differentiated cells while retaining modulated activity. In addition, it was previously unclear whether HLA complex deficient differentiated cells can be reprogrammed to iPSCs and maintained as pluripotent stem cells while having the HLA complex deficiency. Unanticipated failures during cellular reprogramming, maintenance of pluripotency and differentiation may be related to aspects including, but not limited to, development stage specific gene expression or lack thereof, requirements for HLA complex presentation, protein shedding of introduced surface expressing modalities, need for proper and efficient clonal reprogramming, and need for reconfiguration of differentiation protocols.

“Modified HLA deficient iPSC,” as used herein, refers to an HLA deficient iPSC that is further modified by introducing genes expressing proteins related, but not limited, to improved differentiation potential, antigen targeting, antigen presentation, antibody recognition, persistence, immune evasion, resistance to suppression, proliferation, co-stimulation, cytokine stimulation, cytokine production (autocrine or paracrine), chemotaxis, and cellular cytotoxicity, such as non-classical HLA class I proteins (e.g., HLA-E and HLA-G), chimeric antigen receptor (CAR), T cell receptor (TCR), CD16 Fc Receptor, BCL11b, NOTCH, RUNX1, IL15, 4-1BB, DAP10, DAP12, CD24, CD3z, 4-1BBL, CD47, CD113, and PDL1. The cells that are “modified HLA deficient” also include cells other than iPSCs.

“Fc receptors,” abbreviated FcR, are classified based on the type of antibody that they recognize. For example, those that bind the most common class of antibody, IgG, are called Fc-gamma receptors (FcγR), those that bind IgA are called Fc-alpha receptors (FcαR) and those that bind IgE are called Fc-epsilon receptors (FcεR). The classes of FcRs are also distinguished by the cells that express them (macrophages, granulocytes, natural killer cells, T and B cells) and the signalling properties of each receptor. Fc-gamma receptors (FcγR) include several members, FcγRI (CD64), FcγRIIA (CD32), FcγRIIB (CD32), FcγRIIIA (CD16a), FcγRIIIB (CD16b), which differ in their antibody affinities due to their different molecular structures.

CD16 has been identified as two isoforms, Fc receptors FcγRIIIa (CD16a) and FcγRIIIb (CD16b). CD16a is a transmembrane protein expressed by NK cells, which binds monomeric IgG attached to target cells to activate NK cells and facilitate antibody-dependent cell-mediated cytotoxicity (ADCC). “High affinity CD16,” “non-cleavable CD16,” or “high affinity non-cleavable CD16,” as used herein, refers to a variant of CD16. The wildtype CD16 has low affinity and is subject to ectodomain shedding, a proteolytic cleavage process that regulates the cells surface density of various cell surface molecules on leukocytes upon NK cell activation. F176V and F158V are exemplary CD16 variants having high affinity; whereas S197P variant is an example of non-cleavable version of CD16.

The term “adoptive cell therapy” as used herein refers to a cell-based immunotherapy that, as used herein, relates to the transfusion of autologous or allogeneic lymphocytes, such as CD34 cells, hemogenic endothelium cells, hematopoietic stem or progenitor cells, hematopoietic multipotent progenitor cells, T cell progenitor, NK cell progenitor, T cells, NKT cells, NK cells, B cells, or immune regulatory cells, genetically modified or not, that have been expanded ex vivo prior to said transfusion.

A “therapeutically sufficient amount”, as used herein, includes within its meaning a non-toxic but sufficient and/or effective amount of the particular therapeutic and/or pharmaceutical composition to which it is referring to provide a desired therapeutic effect. The exact amount required will vary from subject to subject depending on factors such as the patient's general health, the patient's age and the stage and severity of the condition. In particular embodiments, a therapeutically sufficient amount is sufficient and/or effective to ameliorate, reduce, and/or improve at least one symptom associated with a disease or condition of the subject being treated.

As used herein, the term “subject” refers to any animal, preferably a human patient, livestock, or other domesticated animal.

As used herein, the terms “treat,” “treatment” and the like, when used in reference to a subject in need of a therapeutic treatment, refer to obtaining a desired pharmacologic and/or physiologic effect, including without limitation achieving an improvement or elimination of the symptoms of a disease. The effect can be prophylactic in terms of completely or partially preventing a disease or symptom thereof and/or can be therapeutic in terms of achieving an improvement or elimination of symptoms, or providing a partial or complete cure for a disease and/or adverse effect attributable to the disease. The term “treatment” includes any treatment of a disease in a mammal, particularly in a human, and includes: (a) preventing the disease from occurring in a subject which can be predisposed to the disease but has not yet been diagnosed as having it; (b) inhibiting the disease, or arresting its development; (c) relieving the disease, or causing regression of the disease, or completely or partially eliminating symptoms of the disease; and (d) restoring the individual to a pre-disease state, such as reconstituting the hematopoietic system.

### I. Compositions for Production of Induced Pluripotent Stem Cells

Previously developed FMM (Fate Maintenance Medium) has achieved satisfying long-term stability of iPSCs reprogrammed from various somatic cells that are not T cells. Thus, various modifications of FMM were carried out to enhance long-term stability and preservation, and to reduce the frequency of karyotype abnormalities of iPSCs reprogrammed from all cell sources, especially from T cells, which have been shown to be a difficult cell type for pluripotent cell generation (TiPSC) and lineage-specific and functional effector cell differentiation therefrom. It has been observed that stressors including, but not limited to, single cell dissociation, clonal expansion, freeze-thaw cycles, vector transfection and electroporation and genomic editing, cause genomic instability of the cells and compromise pluripotency, viability and the differentiation potential of pluripotent cells.

The compositions contemplated herein may include a chemically defined stock basal media and various combinations of small molecules, including small molecules and functional variants thereof, that support both efficient and effective reprogramming using minimal reprogramming factors in a feeder-free environment; and enable single cell culture and expansion of pluripotent cells while maintaining a homogenous and genomically stable pluripotent population in a long term (i.e., more than 25, 30, 35, or 50 or more passages) even when subject to one or more stressors. Moreover, the compositions provided herein, provide for culturing pluripotent cells, including TiPSCs, to a state of reduced spontaneous differentiation and a ground state pluripotency (also called naïve pluripotency), irrespective of genetic background of the non-pluripotent cell or the reprogramming processes from which the pluripotent cells are generated.

The compositions contemplated herein are useful, in part, for the production of industrial- or clinical-grade pluripotent cells having reduced spontaneous differentiation as compared to cells generated or cultured in the absence of the compositions. In one embodiment, non-pluripotent cells are induced to become pluripotent cells and cultured to maintain pluripotency in long-term. In another embodiment, non-pluripotent cells are induced to become pluripotent cells and cultured to achieve and/or maintain reduced spontaneous differentiation as compared to cells cultured in the absence of the compositions. In another embodiment, non-pluripotent cells are induced to become pluripotent cells and cultured to achieve and/or maintain ground state pluripotency.

In various embodiments, the compositions provided herein reduce or prevent karyotype abnormality including trisomy in iPSCs, particularly in iPSCs obtained from reprogramming a T cell, as compared to iPSCs generated or maintained without contact of the compositions. Thus, in various embodiments, the compositions maintain ground state pluripotency, normal karyotypes, and genomic stability of one or more pluripotency cells for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more passages, including any intervening number of passages. In other embodiments, the compositions provided herein maintain reduced spontaneous differentiation in one or more pluripotency cells for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more passages, including any intervening number of passages.

In various embodiments, the chemically defined stock basal media for use in the culture medium of the invention may be any defined basal media suitable for supporting the maintenance and/or growth of stem cells, such as conventional human embryonic stem cell media. Examples of defined basal media which may be used in accordance with embodiments of the invention include, but are not limited to: Dulbecco's Modified Eagle Medium (“DMEM”), Basal Media Eagle (BME), DMEM/F-12 (1:1 DMEM and F-12 vol:vol); Medium 199; F-12 (Ham) Nutrient Mixture; F-10 (Ham) Nutrient Mixture; Minimal Essential Media (MEM), Williams' Media E; and RPMI 1640, all of which are available from Gibco-BRL/Life Technologies, Inc., Gaithersburg, Md., among others. Several versions of many of these media are available, which include, but are not limited to: DMEM 11966, DMEM 10314, MEM 11095, Williams' Media E 12251, Ham F12 11059, MEM-alpha 12561, and Medium-199 11151 (Gibco-BRL/Life Technologies). The culture media may include, for example, one or more of the following: amino acids, vitamins, organic salts, inorganic salts, trace elements, buffering salts, sugars, ATP, and the like.

Small molecules, and classes thereof, for use in the cell culture media according to embodiments of the invention are described more fully below. In one embodiment, the composition comprises a cell culture medium and a TGFβ family protein, a Rho Kinase (ROCK) inhibitor (ROCKi), and a MEK inhibitor (MEKi) and WNT activator. In various embodiments, the composition does not comprise a TGFβ inhibitor (TGFβi). In various embodiments, one or more of the TGFβ family protein, ROCKi, MEKi and WNT activator may be added at one or multiple specific stages during iPSC generation and maintenance for a predetermined duration. Such specific stages during iPSC maintenance include, but are not limited to, single cell dissociation of iPSC colonies, single cell sorting of dissociated iPSCs, iPSC single cell clonal expansion, clonal iPSC master cell bank (MCB) cryopreservation, thawing of iPSC MCB, and optionally additional cryopreserve-thaw cycles of the iPSC MCB. For example, in various embodiments, a TGFβ family protein may optionally be added to the composition at single cell dissociation of iPSC colonies, or at iPSC single cell clonal expansion, or at any stage in between. In various embodiments, the concentration of the MEK inhibitor and/or the WNT activator during iPSC maintenance is reduced as compared to the concentration of the MEK inhibitor and/or the WNT activator that is used for reprogramming a non-pluripotent cell to the iPSC. In some embodiments, the concentration of the MEK inhibitor and/or the WNT activator is at an amount that is about 30/6-60%, for example about 35%-55%, preferably about 40/6-50% of the concentration of the MEK inhibitor and/or the WNT activator that may be used for reprogramming a non-pluripotent cell to the iPSC.

In one embodiment, the composition comprising a cell culture medium further comprises a TGFβ family protein, ROCKi, MEKi and WNT activator, and optionally, LIF and/or bFGF, whereas the composition does not comprise a TGFβ inhibitor; and optionally the addition of any of the TGFβ family protein, ROCKi, MEKi and WNT activator to the composition is stage-specific during iPSC maintenance.

In additional embodiments, the cell culture media comprised in the composition is substantially free of cytokines and/or growth factors, and optionally is a feeder-free environment. In other embodiments, the cell culture media contains supplements such as serums, extracts, growth factors, hormones, cytokines and the like.

In further embodiments, the present invention provides a composition for induced pluripotent stem cell production, which comprises a cell culture medium comprising a ROCKi, a MEKi and a WNT activator, an histone deacetylase (HDAC) inhibitor, and a TGFβ inhibitor, and optionally wherein the addition of any of the ROCKi, MEKi and WNT activator, HDAC inhibitor, and TGFβ inhibitor is stage-specific during a process of reprogramming non-pluripotent cells to iPSCs with increased reprogramming efficiency, as compared to reprogramming using previous compositions. Such specific stages during iPSC reprogramming include, but are not limited to, somatic cell transfection (day 0), exogenous gene expression, increase of heterochromatin, loss of somatic cell identity, and iPSC colony formation. For example, in various embodiments, the HDAC inhibitor may optionally be added at chromatin restructuring, or at around day 2-3 (post transfection) and/or the TGFβ inhibitor may optionally be added at the stage of loss of somatic cell identity, or at around day 6-8 (post transfection). At around day 13-15 post transfection, iPSC colonies are usually formed using the disclosed methods and compositions herein.

In one embodiment, the composition for induced pluripotent stem cell production comprises a cell culture medium comprising a ROCKi, a MEKi and a WNT activator, an HDAC inhibitor, and a TGFβ inhibitor, and optionally, LIF and/or bFGF, and optionally where the addition any of the HDAC inhibitor and TGFβ inhibitor is stage-specific during iPSC reprogramming. In some embodiments, the HDAC inhibitor is valproic acid (VPA) or a functional variant or derivative thereof.

### ROCK Inhibitors

Rho associated kinases (ROCK) are serine/threonine kinases that serve downstream effectors of Rho kinases (of which three isoforms exist—RhoA, RhoB and RhoC). ROCK inhibitors suitable for use in compositions contemplated herein include, but are not limited to, polynucleotides, polypeptides, and small molecules. ROCK inhibitors contemplated herein may decrease ROCK expression and/or ROCK activity. Exemplary ROCK inhibitors include, but are not limited to, antibodies to ROCK, dominant negative ROCK variants, and siRNA and antisense nucleic acids that suppress expression of ROCK. Other exemplary ROCK inhibitors include, but are not limited to: thiazovivin, Y27632, Fasudil, AR122-86, Y27632 H-1152, Y-30141, Wf-536, HA-1077, hydroxyl-HA-1077, GSK269962A, SB-772077-B, N-(4-Pyridyl)-N′-(2,4,6-trichlorophenyl)urea, 3-(4-Pyridyl)-1H-indole, and (R)-(+)-trans-N-(4-Pyridyl)-4-(1-aminoethyl)-cyclohexanecarboxamide.

Exemplary ROCK inhibitors for use in the cell culture medium according to embodiments of the invention include thiazovivin, Y27632, pyrintegrin, Blebbistatin, and functional variants or derivatives thereof. In certain embodiments, the ROCK inhibitor is thiazovivin.

### ERK/MEK Inhibitors

Exemplary inhibitors of the ERK/MEK pathway include, but are not limited to, antibodies to MEK or ERK, dominant negative MEK or ERK variants, and siRNA and antisense nucleic acids that suppress expression of MEK and/or ERK. Other exemplary ERK/MEK inhibitors include, but are not limited to, PD0325901, PD98059, U0126, SL327, ARRY-162, PD184161, PD184352, sunitinib, sorafenib, Vandetanib, pazopanib, Axitinib, GSK1 120212, ARRY-438162, RO5126766, XL518, AZD8330, RDEA1 19, AZD6244, FR180204, PTK787, and functional variants or fragments thereof.

Further illustrative examples of MEK/ERK inhibitors include the following compounds: 6-(4-Bromo-2-chloro-phenylamino)-7-fluoro-3-methyl-3H-benzoimidazole-5-carboxylic acid (2,3-dihydroxy-propoxy)-amide; 6-(4-Bromo-2-chloro-phenylamino)-7-fluoro-3-(tetrahydro-pyran-2-ylm-ethyl)-3H-benzoimidazole-5-carboxylic acid (2-hydroxy-ethoxy)-amide, 1-[6-(4-Bromo-2-chloro-phenylamino)-7-fluoro-3-methyl-3H-benzoimida-zol-5-yl]-2-hydroxy-ethanone, 6-(4-Bromo-2-chloro-phenylamino)-7-fluoro-3-methyl-3H-benzoimidazole-5-carboxylic acid (2-hydroxy-1,1-dimethyl-ethoxy)-amide, 6-(4-Bromo-2-chloro-phenylamino)-7-fluoro-3-(tetrahydro-furan-2-ylm-ethyl)-3H-benzoimidazole-5-carboxylic acid (2-hydroxy-ethoxy)-amide, 6-(4-Bromo-2-fluoro-phenylamino)-7-fluoro-3-methyl-3H-benzoimidazole-5-carboxylic acid (2-hydroxy-ethoxy)-amide, 6-(2,4-Dichloro-phenylamino)-7-fluoro-3-methyl-3H-benzoimidazole-5-carboxylic acid (2-hydroxy-ethoxy)-amide, 6-(4-Bromo-2-chloro-phenylamino)-7-fluoro-3-methyl-3H-benzoimidazole-5-carboxylic acid (2-hydroxy-ethoxy)-amide, referred to hereinafter as MEK inhibitor 1; 2-[(2-fluoro-4-iodophenyl)amino]-N-(2-hydroxyethoxy)-1,5-dimethyl-6-oxo-1,6-dihydropyridine-3-carboxamide; referred to hereinafter as MEK inhibitor 2; and 4-(4-bromo-2-fluorophenylamino)-N-(2-hydroxyethoxy)-1,5-dimethyl-6-oxo-1,6-dihydropyridazine-3-carboxamide or a pharmaceutically acceptable salt thereof.

In some embodiments, the MEK/ERK inhibitor is PD0325901.

### Wnt Activators

As used herein, the terms “Wnt signal-promoting agent,” “Wnt pathway activating agent,” “Wnt activator” or “Wnt pathway agonist,” refers to an agonist of the Wnt signaling pathway, including but not limited to an agonist of one or more of Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt10a, Wnt10b, Wnt11, Wnt14, Wnt15, or Wnt16. Wnt pathway agonists further include, but are not limited to, one or more of the following polypeptides or a fragment thereof: a Dkk polypeptide, a crescent polypeptide, a cerberus polypeptide, an axin polypeptide, a Frzb polypeptide, a T-cell factor polypeptide, or a dominant negative disheveled polypeptide.

Non-limiting examples of Wnt pathway agonists further include one or more of the following: a nucleic acid comprising a nucleotide sequence that encodes a Wnt polypeptide, a polypeptide comprising an amino acid sequence of a Wnt polypeptide, a nucleic acid comprising a nucleotide sequence that encodes an activated Wnt receptor, a polypeptide comprising an amino acid sequence of an activated Wnt receptor, a small organic molecule that promotes Wnt/β-catenin signaling, a small organic molecule that inhibits the expression or activity of a Wnt antagonist, an antisense oligonucleotide that inhibits expression of a Wnt antagonist, a ribozyme that inhibits expression of a Wnt antagonist, an RNAi construct, siRNA, or shRNA that inhibits expression of a Wnt antagonist, an antibody that binds to and inhibits the activity of a Wnt antagonist, a nucleic acid comprising a nucleotide sequence that encodes a β-catenin polypeptide, a polypeptide comprising an amino acid sequence of a β-catenin polypeptide, a nucleic acid comprising a nucleotide sequence that encodes a Lef-1 polypeptide, a polypeptide comprising an amino acid sequence of a Lef-1 polypeptide, and functional variants or fragments thereof.

### GSK-3β Inhibitors

GSK-3β inhibitors are specific exemplary Wnt pathway agonists suitable for use in compositions contemplated herein, and may include, but are not limited to, antibodies that bind GSK-3β, dominant negative GSK-3β variants, and siRNA and antisense nucleic acids that target GSK-3β. Other exemplary GSK-3β inhibitors include, but are not limited to, Kenpaullone, 1-Azakenpaullone, CH1R99021, CHIR98014, AR-A014418, CT 99021, CT 20026, SB216763, AR-A014418, lithium, SB 415286, TDZD-8, BIO, BIO-Acetoxime, (5-Methyl-1H-pyrazol-3-yl)-(2-phenylquinazolin-4-yl)amine, Pyridocarbazole-cyclopenadienylruthenium complex, TDZD-8 4-Benzyl-2-methyl-1,2,4-thiadiazolidine-3,5-dione, 2-Thio(3-iodobenzyl)-5-(l-pyridyl)-[1,3,4]-oxadiazole, OTDZT, alpha-4-Dibromoacetophenone, AR-AO 144-18, 3-(1-(3-Hydroxypropyl)-1H-pyrrolo[2,3-b]pyridin-3-yl]-4-pyrazin-2-yl-pyrrole-2,5-dione; TWS1 19 pyrrolopyrimidine compound, L803 H-KEAPPAPPQSpP-NH2 or its myristoylated form; 2-Chloro-1-(4,5-dibromo-thiophen-2-yl)-ethanone, SB216763, SB415286, and functional variants or fragments thereof. Exemplary GSK3 inhibitors for use in the cell culture media according to embodiments of the invention include CHIR99021, BIO, and Kenpaullone. In some embodiments, the GSK3 inhibitor is CHIR99021.

### TGFβ Receptor/ALK5 Inhibitors

TGFβ receptor (e.g., ALK5) inhibitors can include antibodies to, dominant negative variants of, and antisense nucleic acids that suppress expression of, TGFβ receptors (e.g., ALK5). Exemplary TGFβ receptor/ALK5 inhibitors include, but are not limited to, SB431542, A-83-01, 2-(3-(6-Methylpyridin-2-yl)-1H-pyrazol-4-yl)-1,5-naphthyridine, Wnt3a/BIO, BMP4, GW788388 (-(4-[3-(pyridin-2-yl)-1H-pyrazol-4-yl]pyridin-2-yl}-N-(tetrahydro-2H-pyran-4-yl)benzamide), SM16, IN-1130 (3-((5-(6-methylpyridin-2-yl)-4-(quinoxalin-6-yl)-1H-imidazol-2-yl)methyl)benzamide), GW6604 (2-phenyl-4-(3-pyridin-2-yl-1H-pyrazol-4-yl)pyridine), SB-505124 (2-(5-benzo[1,3]dioxol-5-yl-2-tert-butyl-3H-imidazol-4-yl)-6-methylpyridine hydrochloride) and pyrimidine derivatives. Further, while “an ALK5 inhibitor” is not intended to encompass non-specific kinase inhibitors, an “ALK5 inhibitor” should be understood to encompass inhibitors that inhibit ALK4 and/or ALK7 in addition to ALK5, such as, for example, SB-431542. Without intending to limit the scope of the invention, it is believed that ALK5 inhibitors affect the mesenchymal to epithelial conversion/transition (MET) process. TGFβ/activin pathway is a driver for epithelial to mesenchymal transition (EMT). Therefore, inhibiting the TGFβ/activin pathway can facilitate MET (i.e., reprogramming) process.

It has been shown that inhibition of the TGFβ/activin pathway has similar effects of inhibiting ALK5. Thus, any inhibitor (e.g., upstream or downstream) of the TGFβ/activin pathway can be used in combination with, or instead of, ALK5 inhibitors as described in each paragraph herein. Exemplary TGFβ/activin pathway inhibitors include but are not limited to: TGFβ receptor inhibitors, inhibitors of SMAD 2/3 phosphorylation, inhibitors of the interaction of SMAD 2/3 and SMAD 4, and activators/agonists of SMAD 6 and SMAD 7. Furthermore, the categorizations described below are merely for organizational purposes and one of skill in the art would know that compounds can affect one or more points within a pathway, and thus compounds may function in more than one of the defined categories.

Specific examples of TGFβ receptor inhibitors include but are not limited to SU5416; 2-(5-benzo[1,3]dioxol-5-yl-2-tert-butyl-3H-imidazol-4-yl)-6-methylpyridine hydrochloride (SB-505124); lerdelimumb (CAT-152); metelimumab (CAT-192); GC-1008; ID11; AP-12009; AP-11014; LY550410; LY580276; LY364947; LY2109761; SB-505124; SB-431542; SD-208; SM16; NPC-30345; Ki26894; SB-203580; SD-093; Gleevec; 3,5,7,2′,4′-pentahydroxyflavone (Morin); activin-M108A; P144; soluble TBR2-Fc; and antisense transfected tumor cells that target TGFβ receptors.

Inhibitors of SMAD 2/3 phosphorylation can include antibodies to, dominant negative variants of and antisense nucleic acids that target SMAD2 or SMAD3. Specific examples of inhibitors include PD169316; SB203580; SB-431542; LY364947; A77-01; and 3,5,7,2′,4′-pentahydroxyflavone (Morin). Inhibitors of the interaction of SMAD 2/3 and SMAD4 can include antibodies to, dominant negative variants of and antisense nucleic acids that target SMAD2, SMAD3 and/or SMAD4. Specific examples of inhibitors of the interaction of SMAD 2/3 and SMAD4 include but are not limited to Trx-SARA, Trx-xFoxH1b and Trx-Lef1. Activators/agonists of SMAD 6 and SMAD 7 include but are not limited to antibodies to, dominant negative variants of and antisense nucleic acids that target SMAD 6 or SMAD 7.

### HDAC Inhibitors

Exemplary HDAC (histone deacetylase) inhibitors can include antibodies that bind to, dominant negative variants of, and siRNA and antisense nucleic acids that target HDAC. Histone acetylation is involved in histone and DNA methylation regulation. In general at the global level, pluripotent cells have more histone acetylation, and differentiated cells have less histone acetylation. HDAC inhibitors facilitate activation of silenced pluripotency genes. HDAC inhibitors include, but are not limited to, TSA (trichostatin A), VPA (valproic acid), sodium butyrate (NaB), SAHA (suberoylanilide hydroxamic acid or vorinostat), sodium phenylbutyrate, depsipeptide (FR901228, FK228), trapoxin (TPX), 20 cyclic hydroxamic acid-containing peptide 1 (CHAP I), MS-275, LBH589 and PXDIOI.

### Cytokines and Growth Factors

In particular embodiments, the compositions and/or cell culture media of the invention are substantially free of cytokines and/or growth factors. In certain embodiments, the cell culture media contains one or more supplements including, but not limited to sera, extracts, growth factors, hormones, cytokines and the like, which may be added in a stage-specific manner to improve the quality and the efficiency of the reprogramming and/or maintenance process.

Various growth factors and their use in culture media are known and include, for example, ECM proteins, laminin 1, fibronectin, collagen IV isotypes, proteases, protease inhibitors, cell surface adhesion proteins, cell-signaling proteins, cadherins, chloride intracellular channel 1, transmembrane receptor PTK7, insulin-like growth factor, Inhibin beta A, inducers of the TGFβ/Activin/nodal signaling pathway, and Activin A. Cytokines used in the culture media may include, for example, one or more of the following: growth factors such as epidermal growth factor (EGF), acidic fibroblast growth factor (aFGF), basic fibroblast growth factor (bFGF), hepatocyte growth factor (HGF), insulin-like growth factor 1 (IGF-1), insulin-like growth factor 2 (IGF-2), keratinocyte growth factor (KGF), nerve growth factor (NGF), platelet-derived growth factor (PDGF), transforming growth factor beta (TGF-β), leukemia inhibitory factor (LIF), vascular endothelial cell growth factor (VEGF) transferrin, various interleukins (such as IL-1 through IL-18), various colony-stimulating factors (such as granulocyte/macrophage colony-stimulating factor (GM-CSF)), various interferons (such as IFN-γ) and other cytokines having effects upon stem cells such as stem cell factor (SCF) and erythropoietin (Epo).

In particular embodiments, the compositions and/or culture media may include a protein of the TGFβ family as the cytokine/growth factor component of the composition. Examples of TGFβ family proteins include, but are not limited to, Activin A, TGFβ, nodal and functional variants or fragments thereof. These cytokines/growth factors may be obtained commercially, and may be either natural or recombinant. In some embodiments, for culture of a wide variety of mammalian cells, the basal media will contain FGF at a concentration of about 0.01-100 ng/ml, about 0.2-20 ng/ml, and in particular embodiments about 0.5-10 ng/ml. Other cytokines, if used, may be added at concentrations that are determined empirically or as guided by the established cytokine art.

### H. Improved Reprogramming and Maintenance System and the Cells Generated Therefrom

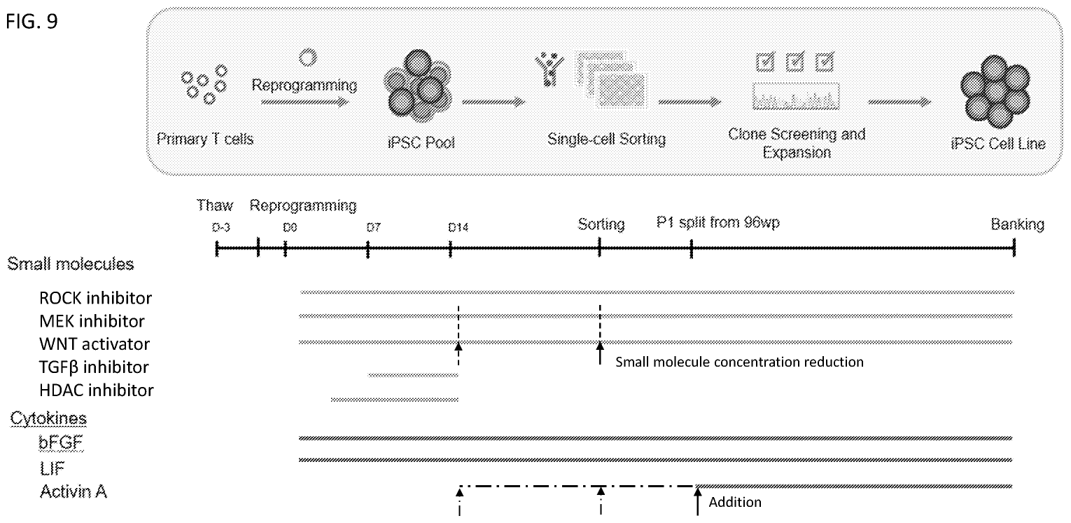

Generally, the present disclosure provides an improved reprogramming process initiated by contacting non-pluripotent cells with at least one reprogramming factor, and optionally in the presence of a combination of a ROCK inhibitor, a MEK inhibitor and a Wnt activator, and optionally a TGFβ inhibitor and/or an HDAC inhibitor, where one or both of the TGFβ inhibitor and HDAC inhibitor are added at one or more selected stages of reprogramming (e.g., FRM2; Tables 1 and 2; see also FIG. 9). The present disclosure also provides an improved maintenance process for iPSCs subject to one or more stressors, where the cells are provided in contact with a combination of a ROCK inhibitor, and a MEK inhibitor and a WNT activator, and a TGFβ family protein optionally at one or more selected stages, where the composition does not comprise a TGFβ inhibitor (e.g., FMM2; Tables 1 and 2; see also FIG. 9).

TABLE 1
Conventional Media and Fate Two-Stage Media
for iPSC Reprogramming and Maintenance

| Conventional hESC | Fate Reprogramming | Fate Maintenance |
|---|---|---|
| Medium (Conv.) | Medium (FRM) | Medium (FMM) |
| DMEM/F12 | DMEM/F12 | DMEM/F12 |
| Knockout Serum | Knockout Serum | Knockout Serum |
| | N2 | |
| | B27 | |
| Glutamine | Glutamine | Glutamine (1x) |
| Non-Essential | Non-Essential | Non-Essential |
| Amino Acids | Amino Acids | Amino Acids |
| β-mercaptoethanol | β-mercaptoethanol | β-mercaptoethanol |
| bFGF (0.2-50 | bFGF (2-500 | bFGF (2-500 |
| ng/ml) | ng/ml) | ng/ml) |
| | LIF (0.2-50 ng/mL) | LIF (0.2-50 ng/ml) |
| | Thiazovivin (0.1-25 μM) | Thiazovivin (0.1-25 μM) |
| | PD0325901 | PD0325901 |
| | (0.005-2 μM) | (0.005-2 μM) |
| | CHIR99021 (0.02-5 μM) | CHIR99021 (0.02-5 μM) |
| | SB431542 (0.04-10 μM) | |

| | |
|---|---|
| In combination | Feeder-free, in combination with |
| with MEF | Matrigel ™ or Vitronectin |

TABLE 2
Improved Fate Stage-Specific Media for
iPSC Reprogramming and Maintenance

| | | |
|---|---|---|
| | Fate | |
| | Reprogramming | Fate Maintenance |
| Stage of Processes | Medium (FRM2) | Medium (FMM2) |

Reprogramming

| | |
|---|---|
| Somatic Cell | FRM (see Table 1) |
| Transfection (day 0) | without TGFβ |
| | inhibitor |
| Chromatin Restructuring | add HDAC inhibitor |
| (around day 2-3 post | (Valproic acid) |
| transfection) | |
| Loss of Somatic Cell | add TGFβ inhibitor |
| Identity (around day 6-8 | |
| post transfection) until | |
| iPSC colony formation | |
| (around day 13-15) | |

iPSC Maintenance

| | |
|---|---|
| iPSC colony dissociation | FMM (see Table 1), |
| | optionally with reduced |
| | concentrations of one or |
| | both of GSK3 inhibitor |
| | and MEK inhibitor, and |
| | optionally add Activin A |
| Single cell sorting | FMM, optionally with |
| | reduced concentrations of |
| | one or both of GSK3 |
| | inhibitor and MEK |
| | inhibitor and optionally |
| | add Activin A |
| Clonal expansion, | FMM, with reduced |
| banking, cryopreserve, | concentrations of one or |
| and thaw | both of GSK3 inhibitor |
| | and MEK inhibitor, and |
| | with Activin A |

### Reprogramming of Cells

One method of obtaining footprint-free iPSCs (i.e., the iPSC does not retain any exogenous reprogramming factor polynucleotides) is to use a plasmid system that mediates transient and temporal transgene expression. One exemplary plasmid system for reprogramming comprises one or more first plasmids carrying a replication origin and polynucleotides encoding reprogramming factor(s) but without EBNA, and a second plasmid comprising EBNA encoding polynucleotides but without a replication origin or reprogramming factor encoding sequences (see, e.g., “STTR system” in U.S. Application Pub. No. 20200270581, the relevant disclosure of which is incorporated herein by reference).

The combination of the plasmids enables cytoplasmic expression of transgenes (EBNA and exogenous reprogramming factors) temporally in the cell upon transduction, and generates a population of EBNA-free intermediary cells, also called reprogramming cells, that present a transitional morphology, or a morphological change (for example mesenchymal to epithelial transition (MET)), but lacks any pluripotent cell morphology or endogenous pluripotency gene expression, such as OCT4, yet are capable of entering a stable self-sustaining pluripotent state. The reprogramming cell, as described herein, also differs from the somatic cell prior to the introduction of the reprogramming factors not only morphologically but functionally as well, in that it is capable of reprogramming to a pluripotent state given a sufficient time period under a culture condition that supports the continuing of the reprogramming process (for example, conventional hES medium, FRM, or FRM2). In some embodiments, the EBNA-free reprogramming cells are transgene-free, and the resultant iPSCs are therefore footprint-free without the need of either selection against EBNA, or continuous passaging in order to eliminate EBNA and transgenes as is often required in episomal reprogramming.

Other than using the plasmid system above, the exogenous reprogramming factors may also be introduced by adenoviral transduction (Zhou et al., Stem Cells (2009); 27:2667-2674), Sendai viral vectors (Fusaki et al., Proc Jpn Acad Ser B Phys Biol Sci. (2009); 85:348-362; Seki et al., Cell Stem Cell (2010); 7:11-14; Ban et al., PNAS (2011); 108:14234-14239), minicircle DNA vectors (Okita et al., Science (2008); 322:949-953), and oriP/EBNA episomal vectors (Malik et al., Methods Mol Biol. (2013); 997:23-33).

Reprogramming factors known for stem cell reprogramming in the field could all be used with the present reprogramming system and method. In one embodiment, the reprogramming factors include, but are not limited to, OCT4, YAP1, SOX2 and large T antigen (LTag). Additional reprogramming factors include but are not limited to, NANOG, KLF, LIN28, MYC, ECAT1, UTF1, ESRRB, HESRG, CDH1, TDGF1, DPPA4, DNMT3B, ZIC3, and L1TD1. In another embodiment, the reprogramming factors include, but are not limited to, OCT4, YAP1, SOX2, large T antigen, MYC, LIN28, ESRRB and ZIC3 for somatic cell reprogramming, and for T cell reprogramming without using KLF. Polynucleotides encoding these reprogramming factors may be comprised in the same plasmid construct containing oriP but not EBNA (i.e., the same first plasmid). Polynucleotides encoding these reprogramming factors may be comprised in at least two plasmid constructs each containing oriP but not EBNA (i.e., multiple first plasmids). In various embodiments, the polynucleotides encoding these reprogramming factors are comprised in four plasmid constructs each containing oriP but not EBNA (i.e., four first plasmids).

Polynucleotides encoding these reprogramming factors may be comprised in a polycistronic construct (i.e., multiple coding sequences controlled by one promoter) or non-polycistronic construct (multiple coding sequences with some controlled by one promoter and some by a different promoter). The promoter may be, for example, CMV, EF1α, PGK, CAG, UBC, and other suitable promoters that are constitutive, inducible, endogenously regulated, or temporal-, tissue- or cell type-specific. In one embodiment, the promoter is CAG. In another embodiment, the promoter is EF1α. In some embodiments, the polycistronic construct may provide a single open reading frame (for example, multiple coding sequences are operatively linked by a self-cleaving peptide encoding sequence such as 2A) or multiple open reading frames (for example, multiple coding sequences linked by an Internal Ribosome Entry Site, or IRES).

In some embodiments of the plasmid system of the present invention, one or more plasmid constructs (first plasmids) collectively comprise polynucleotides encoding one or more reprogramming factors selected from the group consisting of OCT4, YAP1, SOX2 and large T antigen (LTag). In additional embodiments, the one or more plasmid constructs (first plasmids) collectively further comprise polynucleotides encoding one or both of SOX2 and KLF, or one or more of MYC, LIN28, ESRRB and ZIC3. In some embodiments, only one first plasmid construct is in the system and provides all selected reprogramming factors. In some other embodiments, there are two or more first plasmid constructs in the system that provide one or more reprogramming factors, with each construct comprising the same or different reprogramming factors encoded by at least one copy of polynucleotide. In some embodiments, the one or more first plasmid constructs collectively comprise at least two polynucleotides encoding OCT4, and one or more polynucleotides encoding at least one of YAP1, SOX2, LTag, ECAT1, UTF1, ESRRB, HESRG, CDH1, TDGF1, DPPA4, DNMT3B, ZIC3, and L1TD1.

When a first plasmid construct comprises more than one polynucleotide encoding more than one reprogramming factor, the adjacent polynucleotides are operatively connected by a linker sequence encoding a self-cleaving peptide or an IRES. The self-cleaving peptide may be a 2A peptide. The 2A peptides may be derived from FMDV (foot-and-mouth disease virus), ERAV (equine rhinitis A virus), PTV-1 (porcine tescho virus-1), or TaV (thosea asigna virus), which are referred to as F2A, E2A, P2A and T2A, respectively. The multiple 2A peptides in a first plasmid construct may be the same or different. In some embodiments, two closest neighboring 2A peptides are different, for example: RF-2A1-RF-2A2-RF-2A1, where 2A1 and 2A2 are different.

A library of first plasmid constructs can be pre-constructed, with each construct containing one or more polynucleotides that encode various number, type and/or combinations of reprogramming factors. Reprogramming is known to be an inefficient and stochastic process with long latency. The timing and levels of expression, and the stoichiometry of reprogramming factors drive reprogramming kinetics in different phases of reprogramming and intermediate states of the cells undergoing reprogramming and determine the completion of reprogramming. Reprogramming factor stoichiometry also affects reprogramming efficiency, and produces iPSCs with varied quality, such as primed versus ground state pluripotency, and related biological properties including clonality, self-renewal, homogeneity, and pluripotency maintenance (as opposed to spontaneous differentiation) of the iPSCs. Stoichiometry measures the quantitative relationships between reagents in a reaction process, and is used to determine the amount of reagents that are needed in a given reaction, and sometimes the amount of products produced. Stoichiometry considers both stoichiometric amount of a reagent or stoichiometric ratio of reagents, which is the optimum amount or ratio of reagent(s) to complete the reaction. One aspect of the application provides a system and method to evaluate or utilize reprogramming factor stoichiometry by allowing one or more first plasmids to be conveniently selected from the library, mix-and-matched, dosage-adjusted, and co-transfected.

The second plasmid of the present reprogramming system provides an expression cassette comprising a promoter and an EBNA encoding polynucleotide, wherein neither the expression cassette nor the second plasmid comprises any polynucleotide encoding reprogramming factors. The promoter comprised in the second plasmid may be, for example, CMV, EF1α, PGK, CAG, UBC, and other suitable promoters that are constitutive, inducible, endogenously regulated, or temporal-, tissue- or cell type-specific. In one embodiment, the promoter is CAG. In another embodiment, the promoter is EF1α. By co-transfecting a non-pluripotent cell with the above-described combination of at least one first plasmid and a second plasmid, the stand-alone EBNA and oriP, along with at least one reprogramming factor, are introduced to the non-pluripotent cells to initiate reprogramming.

In some embodiments, the reprogramming is initiated in the presence of a combination of small molecule compounds comprising a ROCK inhibitor, a MEK inhibitor, a WNT activator, an HDAC inhibitor and a TGFβ inhibitor, and iPSCs are generated after a sufficient period of time. In some embodiments, the reprogramming is initiated in the presence of a combination of small molecule compounds comprising a ROCK inhibitor, a MEK inhibitor, a WNT activator, optionally at a stage of exogenous reprogramming factor expression, or at about day 1-2 post reprogramming factor transfer; thereafter, an HDAC inhibitor is added optionally at a stage of chromatin restructuring, or at around day 2-3 post reprogramming factor transfer; and thereafter, a TGFβ inhibitor is added optionally at a stage of loss of somatic cell identity, or at around day 6-8 post transfection, to establish pluripotency and genomic stability of the iPSCs. In some embodiments, the optimized reprogramming compositions and process, including the use of the first and second vectors, the combination of reprogramming factors, and/or the small molecule treatment during one or more selected stages during reprogramming, as disclosed herein, result in reliably footprint-free iPSCs that are produced with a higher efficiency, and have established pluripotency (including naïve pluripotency) and improved genomic stability, as compared to previous systems. Moreover, the iPSCs reprogrammed from T cells using the optimized reprogramming compositions and process as disclosed have much lower propensity to having karyotype abnormalities including trisomy.

In some embodiments, the reprogramming is under a feeder-free condition. In particular embodiments, the feeder-free environment is essentially free of human feeder cells and is not pre-conditioned by feeder cells, including without limitation, mouse embryonic fibroblasts, human fibroblasts, keratinocytes, and embryonic stem cells.

### Maintenance of Cells

Among the many key aspects in cell therapy manufacturing processes, cell expansion and cryopreservation have been identified as critical areas of interest, where cell viability and functionality are profoundly impacted during the freeze-thaw cycle, and in vivo cell efficacy and persistency of effector cells derived from iPSC differentiation are intricately affected during effector cell expansion stage after iPSC differentiation. Thus, another aspect of the present invention addresses long-term preservation of induced pluripotent stem cells (iPSCs), especially in feeder cell-free conditions, when the cells undergo one or more stressors including, but not limited to, single cell dissociation, clonal expansion, freeze-thaw cycles, vector transfection and electroporation and genomic editing. In some embodiments, the genomic editing of iPSC is a multiplex editing.

Accordingly, in some embodiments, the cells after being induced for about 7 to 35 days, about 10 to 32 days, about 15 to 31 days, about 17 to 29 days, about 19 to 27 days, or about 21 to about 25 days are optionally subject to disassociation, such that the cells are dissociated into a single cell suspension, either by enzymatic or mechanical means. The dissociated cells may be resuspended in any suitable solution or media for maintaining cells or performing cell sorting. In some embodiments, the single dissociated cell suspension comprises a TGFβ family protein, a ROCK inhibitor and a MEK inhibitor and a WNT activator. In some embodiments, the TGFβ family protein is optionally added at single cell dissociation of the iPSC colony or at iPSC single cell expansion, or any stage in between. In particular embodiments, WNT activator comprises a GSK3 inhibitor and/or the TGFβ family protein comprises at least one of Activin A, TGF$, nodal, and functional variants or fragments thereof. In certain embodiments, the GSK3 inhibitor is CHIR99021, the MEK inhibitor is PD0325901, and/or the Rock inhibitor is thiazovivin.

In some embodiments, the single dissociated cell suspension may be further sorted. In various embodiments, enrichment provides a method for deriving clonal iPSC colonies in a relatively short time, thereby improving the efficiency of iPSC generation. Enrichment may comprise sorting a population of cells by identifying and obtaining cells expressing markers of pluripotency, thereby obtaining a population of enriched pluripotent cells. An additional enrichment methodology comprises the depletion of cells expressing markers of differentiation, non-reprogrammed or non-pluripotent cells. In some embodiments, the cells for sorting are pluripotent cells. In some embodiments, the cells for sorting are reprogramming cells. In some embodiments, the cells for sorting have been induced to reprogram for at least 1, 2, 3, 4, 5, 6, 7, 8 or more days, but no more than 25, 26, 28, 30, 32, 35, 40 days, or any number of days in between. In some embodiment, the cells for sorting have been induced to reprogram for about 21 to 25 days, about 19 to 23 days, about 17 to 21 days, about 15 to about 19, or about 16 to about 18 days.

Cells may be sorted by any suitable method of sorting cells, such as by magnetic bead or flow cytometry (FACS) sorting. Cells may be sorted based on one or more markers of pluripotency, including without limitation, expression of SSEA3/4, TRA1-60/81, TRA1-85, TRA2-54, GCTM-2, TG343, TG30, CD9, CD29, CD133/prominin, CD140a, CD56, CD73, CD105, OCT4, NANOG, SOX2, KLF4, SSEA1 (Mouse), CD30, SSEA5, CD90 and/or CD50. In various embodiments, cells are sorted based on at least two, at least three, or at least four markers of pluripotency. In certain embodiments, cells are sorted based on expression of SSEA4, and in certain particular embodiments based on expression of SSEA4 in combination with TRA1-81 and/or TRA1-60. In certain embodiments, cells are sorted based on SSEA4, TRA1-81, or TRA1-60, and/or CD30 expression. In one embodiment, cells are sorted based on SSEA4, TRA1-81 and CD30. In another embodiment, cells are sorted based on SSEA4, TRA1-60 and CD30. In certain embodiments, cells are initially depleted for non-reprogrammed cells using one or more surface markers of differentiating cells including, but not limited to, CD13, CD26, CD34, CD45, CD31, CD46 and CD7, and then enriched for pluripotent markers such as SSEA4, TRA1-81 and/or CD30.

After reprogramming, the iPSCs are maintained, passaged, expanded, and/or cryopreserved. In some embodiments, the iPSCs are cultured, i.e., maintained, passaged expanded, and cryopreserved, as single cells for an extended period in the maintenance medium, for example, the FMM2 as shown in Table 2. The iPSCs cultured in FMM2 have been shown to continue to maintain their undifferentiated, and ground or naïve, profile; genomic stability without the need for culture cleaning or selection; and readily give rise to all three somatic lineages, in vitro differentiation via embryoid bodies or monolayer (without formation of embryoid bodies); and in vivo differentiation by teratoma formation. See, for example, International Pub. No. WO 2015/134652 and U.S. Application Pub. No. 20170073643, the disclosure of each of which is incorporated herein by reference. In various embodiments, the iPSCs cultured in FMM2 demonstrated increased expression of one or more of naïve-specific markers, including without limitation, TBX3 (T-box transcription factor; UniProt Accession No. 015119), TFCP2L1 (Transcription factor CP2-like protein 1; UniProt Accession No. Q9NZI6), UTF1 (Undifferentiated embryonic cell transcription factor 1; UniProt Accession No. Q5T230), FGF4 (Fibroblast growth factor receptor 4; UniProt Accession No. P22455), TFCP2L1 (Transcription factor CP2-like protein 1; UniProt Accession No. Q9NZI6), PRDM14 (PR domain zinc finger protein 14; UniProt Accession No. Q9GZV8), DPPA5 (Developmental pluripotency-associated 5 protein; UniProt Accession No. A6NC42), DNMT3L (DNA (cytosine-5)-methyltransferase 3-like; UniProt Accession No. Q9UJW3), KLF4 (Krueppel-like factor 4; UniProt Accession No. 043474), and MAEL (Protein maelstrom homolog; UniProt Accession No. Q96JY0), as compared to expression with previous systems. The cells suitable for reprogramming using the present reprogramming system and method generally include any non-pluripotent cells. Non-pluripotent cells include, but are not limited to, terminally differentiated cells; or multipotent or progenitor cells, which are not able to give rise to all three types of germ layer lineage cells. In some embodiments, the non-pluripotent cell for reprogramming is a primary cell, i.e., a cell isolated directly from human or animal tissue. In some embodiments, the non-pluripotent cell for reprogramming is a source specific cell, for example, donor-, disease-, or treatment response-specific. In some embodiments, the non-pluripotent cell for reprogramming is a primary immune cell. In some embodiments, the non-pluripotent cell for reprogramming is itself derived from a pluripotent cell, including embryonic stem cell and induced pluripotent stem cell. In some embodiments, the non-pluripotent cell for reprogramming is a derived immune effector cell, for example, an iPSC-derived non-natural T- or NK-like cell.

In some other embodiments, the non-pluripotent cell for reprogramming is a genomically modified primary or derived cell. The genetic modification comprised in the non-pluripotent cell include insertion, deletion or substitution in the genome, which leads to knock-in, knock-out or knock-down of a gene expression. The modified expression in the non-pluripotent cell for reprogramming may be constitutive or inducible (for example, development stage-, tissue-, cell-, or inducer-specific). In some embodiments, the insertion or substitution is a locus specific targeted integration. In some embodiments, the selected locus for integration is a safe harbor locus or an endogenous gene locus of interest. Safe harbor loci may include AAVS1, CCR5, ROSA26, collagen, HTRP, H11, beta-2 microglobulin, GAPDH, TCR or RUNX1, and other loci meeting the criteria of a genome safe harbor. For an integration site to be a potential safe harbor locus, it ideally needs to meet criteria including, but not limited to: absence of disruption of regulatory elements or genes, as judged by sequence annotation; is an intergenic region in a gene dense area, or a location at the convergence between two genes transcribed in opposite directions; keep distance to minimize the possibility of long-range interactions between vector-encoded transcriptional activators and the promoters of adjacent genes, particularly cancer-related and microRNA genes; and has apparently ubiquitous transcriptional activity, as reflected by broad spatial and temporal expressed sequence tag (EST) expression patterns, indicating ubiquitous transcriptional activity. This latter feature is especially important with regard to pluripotent cells, where during differentiation, chromatin remodeling typically leads to silencing of some loci and potential activation of others. Within the region suitable for exogenous insertion, a precise locus chosen for insertion should be devoid of repetitive elements and conserved sequences and to which primers for amplification of homology arms could easily be designed. In one example, the non-pluripotent cell for reprogramming using the present system and method is a T cell comprising a CAR at the endogenous TCR locus, and the TCR expression is disrupted as a result of the CAR integration.

In one embodiment, reprogramming of a genetically modified non-pluripotent cell is to obtain a genome engineered iPSC comprising the same genetic modification(s). As such, in some other embodiments, one or more such genomic editing may be introduced to the iPSC after reprogramming to obtain a genome-engineered iPSC. In one embodiment, the iPSC for genomic editing is a clonal line or a population of clonal iPS cells.

In some embodiments, the genome-engineered iPSCs comprising one or more targeted genetic edits are maintained, passaged, expanded and/or cryopreserved in a medium comprising a TGFβ family protein, a ROCKi, and a MEKi and a WNT activator, and is free of, or essentially free of, TGFβ receptor/ALK5 inhibitors, wherein the iPSCs retain the intact and functional targeted editing at the selected sites. In some embodiments, the TGFβ family protein, ROCKi, and/or MEKi and WNT activator are added at specific stages during the iPSC maintenance. In some embodiments, the iPSC maintenance includes, but is not limited to, one or more of sequential stages comprising: single cell dissociation of iPSC colony, single cell sorting of dissociated iPSCs, iPSC single cell clonal expansion, clonal iPSC master cell bank (MCB) cryopreservation, thawing of iPSC/MCB, and optionally additional cryopreserve-thaw cycles of the iPSC MCB. In some embodiments, addition of the TGFβ family protein optionally occurs at single cell dissociation of the iPSC colony, at iPSC single cell clonal expansion, or at any stage in between. In some embodiments, the MEKi and/or the WNT activator is present in an amount that is about 30-60% that used for reprogramming a non-pluripotent cell.

In some embodiments, the genetic editing introduces one or more of a safety switch protein, a targeting modality, a receptor, a signaling molecule, a transcription factor, a pharmaceutically active protein or peptide, a drug target candidate, and a protein promoting engraftment, trafficking, homing, tumor infiltration, viability, self-renewal, persistence, and/or survival of the pluripotent cell and/or derivative cells thereof. In one embodiment, the genome engineered iPSC comprises one or more suicide gene mediated safety switch including, without limitation, caspase 9 (or caspase 3 or 7), thymidine kinase, cytosine deaminase, B-cell CD20, modified EGFR, and any combination thereof. In some embodiments, the genomically engineered iPSCs have at least one genomic modification comprising introduced or increased expression of a chimeric receptor, a homing receptor, an anti-inflammatory molecule, an immune checkpoint protein, a cytokine/chemokine decoy receptor, a growth factor, an altered pro-inflammatory cytokine receptor, a CAR, or a surface triggering receptor for coupling with bi- or multi-specific or universal engagers; or reduced or silenced expression of a co-stimulatory gene. In some embodiments, the genome-engineered iPSCs comprise a high affinity and/or non-cleavable CD16 as a targeting modality. In some other embodiments, the targeting modality comprised in the genome-engineered iPSCs is a chimeric antigen receptor (CAR) that is T cell specific, or NK cell specific, or compatible to both T and NK cells.

In some embodiments, the genome-engineered iPSC comprises one or more exogenous polynucleotides or in/dels in one or more endogenous genes. In some embodiments, the in/del comprised in an endogenous gene results in disruption of gene expression. In some embodiments, the in/del comprised in an endogenous gene results in knock-out of the edited gene. In some embodiments, the in/del comprised in an endogenous gene results in knock-down of the edited gene. In some embodiments, the genome-engineered iPSC comprising one or more exogenous polynucleotides at selected site(s) may further comprise one or more targeted editing including in/dels at selected site(s). In some embodiments, the in/del is comprised in one or more endogenous genes associated with immune response regulation and mediation. In some embodiments, the in/del is comprised in one or more endogenous checkpoint genes. In some embodiments, the in/del is comprised in one or more endogenous T cell receptor genes. In some embodiments, the in/del is comprised in one or more endogenous MHC class I suppressor genes. In some embodiments, the in/del is comprised in one or more endogenous genes associated with the major histocompatibility complex. In one embodiment, the modified iPSC cells comprise a deletion, disruption, or reduced expression in at least one of B2M, TAP1, TAP2, Tapasin, NLRC5, RFXANK, CIITA, RFX5, RFXAP, and any of the HLA genes in the chromosome 6p21 region. In another embodiment, the modified iPS cells comprise introduction of HLA-E or HLA-G. In yet some other embodiments, the genome-engineered iPS cells comprise an interrupted TCR locus.

The various targeted genetic editing methods of iPSCs, especially for effectively engineer iPSC at a single cell level with multi-gene at multi-loci targeting strategies include those depicted in, for example, International Pub. No. WO 2017/079673, the disclosure of which is incorporated herein by reference.

III. iPSC Derivative Cells Obtained In Vitro

The present invention further provides, in some embodiments, non-pluripotent cells derived from the iPSCs obtained using the system and methods as disclosed herein. In some embodiments, the iPSCs for generating derivative non-pluripotent cells are genome-engineered, either through targeted editing of iPSCs, or through reprogramming genome-engineered non-pluripotent cells having site specific integration or in/dels. In some embodiments, the iPSC-derived non-pluripotent cells are progenitor cells or fully differentiated cells. In some embodiments, the iPSC-derived non-pluripotent cells are immune effector cells. In some embodiments, the iPSC-derived cells retaining the same targeted editing comprised in the genome-engineered iPSC are non-natural mesodermal cells, CD34 cells, hemogenic endothelium cells, hematopoietic stem or progenitor cells, hematopoietic multipotent progenitor cells, T cell progenitors, NK cell progenitors, T cells, NKT cells, NK cells, B cells, immune regulatory cells or any desired cell of any germ layer lineage. In some embodiments, the iPSC-derived non-natural immune regulatory cells comprise myeloid-derived suppressor cells (MDSCs), regulatory macrophages, regulatory dendritic cells or mesenchymal stromal cells, which are potent immune regulators of NK, B, and T cells.

In addition to producing unlimited numbers of cells of a certain type or subtype that are hard to come by through isolation from donor sources, it has been shown that human iPSC-derived lineages exhibit the properties of fetal-stage cells, such that the reprogramming process resets not only cell fate (from specified/differentiated to pluripotent) but also the chronological age characteristic of the donor cell population independent of the age of the initial somatic cell donor. Other than fetal-like properties observed in iPSC-derived lineages including neural, cardiac, or pancreatic cells, cellular hallmarks of aging have shown measurable changes indicative of rejuvenation of redifferentiated cells from iPSC following the reprogramming process. Age-related parameters expressed in the aged donor fibroblast population were reset after iPSC induction and differentiation into iPSC-derived fibroblast-like cells (Miller et al., 2013). iPSC-derived antigen-specific T cells differentiated from iPSCs reprogrammed from a T cell clone demonstrate rejuvenation through elongated telomeres than those in the original T cell clone. Additional changes in fully differentiated cells indicative of a rejuvenation process include, but are not limited to, global increase of heterochromatin, improved mitochondrial function (ROS reduction, reduced mtDNA mutation, presence of ultrastructure), increased DNA damage responses, telomere elongation and decrease of percentage of short telomere, and decrease in the fraction of senescent cells. (Nishimura et al., 2013). The positive reset in these various age-related aspects lead to a non-natural cell having a higher potential for proliferation, survival, persistence, and memory like functions. Hence, the reprogramming and redifferentiation mediated rejuvenation imparts many molecular, phenotypic and functional properties in a fully differentiated iPSC-derived cell, which non-natural properties set it apart from its primary-cell counterpart despite their likeness in cell lineage.

Applicable differentiation methods and compositions for obtaining iPSC-derived hematopoietic cell lineages include those depicted in, for example, International Pub. No. WO 2017/078807, the disclosure of which is incorporated herein by reference. As provided herein, the methods and compositions for generating hematopoietic cell lineages are through definitive hemogenic endothelium (HE) derived from pluripotent stem cells, including iPSCs under serum-free, feeder-free, and/or stromal-free conditions and in a scalable and monolayer culturing platform without the need of EB formation. Cells that may be differentiated according to the provided methods range from pluripotent stem cells, to progenitor cells that are committed to a particular terminally differentiated cell and transdifferentiated cells, cells of various lineages directly transitioned to hematopoietic fate without going through a pluripotent intermediate. Similarly, the cells produced by differentiation of stem cells range from multipotent stem or progenitor cells to terminally differentiated stem cells, and all intervening hematopoietic cell lineages.

The methods for differentiating and expanding cells of the hematopoietic lineage from pluripotent stem cells in monolayer culturing comprise contacting the pluripotent stem cells with a BMP pathway activator, and optionally, bFGF. As provided, the pluripotent stem cell-derived mesodermal cells are obtained and expanded without forming embryoid bodies from pluripotent stem cells. The mesodermal cells are then subjected to contact with a BMP pathway activator, bFGF, and a WNT pathway activator to obtain expanded mesodermal cells having definitive hemogenic endothelium (HE) potential without forming embryoid bodies from the pluripotent stem cells. By subsequent contact with bFGF, and optionally, a ROCK inhibitor, and/or a WNT pathway activator, the mesodermal cells having definitive HE potential are differentiated to definitive HE cells, which are also expanded during differentiation.

The methods provided herein for obtaining cells of the hematopoietic lineage are superior to EB-mediated pluripotent stem cell differentiation, because EB formation leads to modest to minimal cell expansion, does not allow monolayer culturing which is important for many applications requiring homogeneous expansion, and homogeneous differentiation of the cells in a population, and is laborious and low efficiency.

The provided monolayer differentiation platform facilitates differentiation towards definitive hemogenic endothelium resulting in the derivation of hematopoietic stem cells and differentiated progeny such as T, B, NKT, NK like cells, and regulatory cells. The monolayer differentiation strategy combines enhanced differentiation efficiency with large-scale expansion enables the delivery of therapeutically relevant number of pluripotent stem cell-derived hematopoietic effector cells for various therapeutic applications. Further, the monolayer culturing using the methods provided herein leads to functional hematopoietic lineage cells that enable full range of in vitro differentiation, ex vivo modulation, and in vivo long term hematopoietic self-renewal, reconstitution and engraftment. As provided, the iPSC-derived hematopoietic lineage cells include, but are not limited to, definitive hemogenic endothelium, hematopoietic multipotent progenitor cells, hematopoietic stem and progenitor cells, T cell progenitors, NK cell progenitors, and immune effector cells having the functions of T cells, NK cells, NKT cells, B cells, macrophages, neutrophils, myeloid-derived suppressor cells (MDSCs), regulatory macrophages, regulatory dendritic cells, mesenchymal stromal cells, or any combination thereof.

The method for directing differentiation of pluripotent stem cells into cells of a definitive hematopoietic lineage, wherein the method comprises: (i) contacting pluripotent stem cells with a composition comprising a BMP activator, and optionally bFGF, to initiate differentiation and expansion of mesodermal cells from the pluripotent stem cells; (ii) contacting the mesodermal cells with a composition comprising a BMP activator, bFGF, and a GSK3 inhibitor, wherein the composition is optionally free of TGFβ receptor/ALK inhibitor, to initiate differentiation and expansion of mesodermal cells having definitive HE potential from the mesodermal cells; (iii) contacting the mesodermal cells having definitive HE potential with a composition comprising a ROCK inhibitor, one or more growth factors and cytokines selected from the group consisting of bFGF, VEGF, SCF, IGF, EPO, IL6, and IL11; and optionally, a Wnt pathway activator, wherein the composition is optionally free of TGFβ receptor/ALK inhibitor, to initiate differentiation and expansion of definitive hemogenic endothelium from pluripotent stem cell-derived mesodermal cells having definitive hemogenic endothelium potential.

In some embodiments, the method further comprises contacting pluripotent stem cells with a composition comprising a ROCK inhibitor, and a MEK inhibitor and WNT activator, and optionally a TGFβ family protein, wherein the composition is free of TGFβ receptor/ALK inhibitors, to seed and expand the pluripotent stem cells with a lower propensity for genomic abnormalities as compared to pluripotent stem cells without contact with the composition. In some embodiments, the pluripotent stem cells are iPSCs, or naïve iPSCs, or iPSCs comprising one or more genetic imprints; and the one or more genetic imprints comprised in the iPSC are retained in the hematopoietic cells differentiated therefrom. In some embodiments of the method for directing differentiation of pluripotent stem cells into cells of a hematopoietic lineage, the differentiation of the pluripotent stem cells into cells of hematopoietic lineage is void of generation of embryoid bodies, and is in a monolayer culturing form.

In some embodiments of the above method, the obtained pluripotent stem cell-derived definitive hemogenic endothelium cells are CD34⁺. In some embodiments, the obtained definitive hemogenic endothelium cells are CD34⁺CD43⁻. In some embodiments, the definitive hemogenic endothelium cells are CD34⁺CD43⁻CXCR4⁻CD73⁻. In some embodiments, the definitive hemogenic endothelium cells are CD34⁺CXCR4⁻CD73⁻. In some embodiments, the definitive hemogenic endothelium cells are CD34⁺CD43⁻CD93⁻. In some embodiments, the definitive hemogenic endothelium cells are CD34⁺CD93⁻. In some embodiments, the definitive hemogenic endothelium cells are CD34⁺CD93⁻CD73⁻.

In some embodiments of the above method, the method further comprises (i) contacting pluripotent stem cell-derived definitive hemogenic endothelium with a composition comprising a ROCK inhibitor; one or more growth factors and cytokines selected from the group consisting of VEGF, bFGF, SCF, FIt3L, TPO, and IL7; and optionally a BMP activator; to initiate the differentiation of the definitive hemogenic endothelium to pre-T cell progenitors; and optionally, (ii) contacting the pre-T cell progenitors with a composition comprising one or more growth factors and cytokines selected from the group consisting of SCF, Flt3L, and IL7, but free of one or more of VEGF, bFGF, TPO, BMP activators and ROCK inhibitors, to initiate the differentiation of the pre-T cell progenitors to T cell progenitors or T cells. In some embodiments of the method, the pluripotent stem cell-derived T cell progenitors are CD34⁺CD45⁺CD7T. In some embodiments of the method, the pluripotent stem cell-derived T cell progenitors are CD45⁺CD7⁺. In some embodiments, the pluripotent stem cell-derived T cell comprise a fraction of γδT cells much higher than primary T cells isolated from donor sources.

In yet some embodiments of the above method for directing differentiation of pluripotent stem cells into cells of a hematopoietic lineage having improved genomic stability, the method further comprises: (i) contacting pluripotent stem cell-derived definitive hemogenic endothelium with a composition comprising a ROCK inhibitor; one or more growth factors and cytokines selected from the group consisting of VEGF, bFGF, SCF, Flt3L, TPO, IL3, 1L7, and 115; and optionally, a BMP activator, to initiate differentiation of the definitive hemogenic endothelium to pre-NK cell progenitor; and optionally, (ii) contacting pluripotent stem cells-derived pre-NK cell progenitors with a composition comprising one or more growth factors and cytokines selected from the group consisting of SCF, Flt3L, IL3, IL7, and IL15, wherein the medium is free of one or more of VEGF, bFGF, TPO, BMP activators and ROCK inhibitors, to initiate differentiation of the pre-NK cell progenitors to NK cell progenitors or NK cells. In some embodiments, the pluripotent stem cell-derived NK progenitors are CD3-CD45⁺CD56⁺CD7⁺. In some embodiments, the pluripotent stem cell-derived NK cells are CD3-CD45⁺CD56⁺. In some embodiments, the pluripotent stem cell-derived NK cells are optionally further defined by one or more of NKp46 (CD335), NKp30 (CD337), DNAM-1 (CD226), 2B4 (CD244), CD57 and CD16.

In another embodiment of the method, the method enables producing immune regulatory cells from contacting pluripotent stem cell-derived definitive HE with a medium comprising a ROCK inhibitor, MCSF, GMCSF, and one or more growth factors and cytokines selected from the group consisting of IL1b, IL3, IL6, IL4, IL10, IL13, TGFβ, bFGF, VEGF, SCF, and FLT3L, and optionally, one or both of an AhR antagonist and a prostaglandin pathway agonist.

In some embodiments, the derived immune regulatory cells comprise myeloid derived suppressor cells (MDSCs). In one embodiment, the population of derived immune regulatory cells comprises CD45⁺CD33⁺ cells. In some embodiments, the population of derived immune regulatory cells comprise monocytes. In some embodiments, the monocytes comprise CD45⁺CD33⁺CD14⁺ cells. In yet some other embodiments, the population of derived immune regulatory cells comprise CD45⁺CD33⁺PDL1⁺ cells. One aspect of the invention provides an enriched cell population or subpopulation of iPSC-derived immune regulatory cells comprising CD45⁺CD33⁺, CD45⁺CD33⁺CD14⁺, or CD45⁺CD33⁺PDL1⁺ cells. In some other embodiments, the population of derived immune regulatory cells comprise CD33⁺CD15⁺CD14⁻CD11b⁻ cells. In some embodiments, the population of derived immune regulatory cells comprising iMDSCs comprise less than about 50%, about 40%, about 30%, about 20%, about 10%, about 5%, about 2%, about 1%, about 0.1% of erythrocytes, lymphoid, granulocytes, CD45⁻CD235⁺ cells, CD45⁺CD7⁺cells, or CD45⁺CD33⁺CD66b⁺ cells. In some embodiments, the population of derived immune regulatory cells is essentially free of erythrocytes, lymphoid, granulocytes, CD45⁻CD235′ cells, CD45⁺CD7⁺ cells, or CD45⁺CD33⁺CD66b⁺ cells.

III. Therapeutic Use of iPSCs and Derivative Immune Cells Therefrom

The present invention provides, in some embodiments, a composition comprising an isolated population or subpopulation of iPSCs and/or immune cells that have been derived from said iPSC using the methods and compositions as disclosed. In some embodiments, the iPSCs comprise one or more targeted genetic edits which are retainable in the iPSC-derived immune effector cells, wherein the genetically engineered iPSCs and derivative cells thereof are suitable for cell-based adoptive therapies. In one embodiment, the isolated population or subpopulation of genetically engineered immune cell comprises iPSC-derived HSC cells. In one embodiment, the isolated population or subpopulation of genetically engineered immune cell comprises iPSC-derived HSC cells. In one embodiment, the isolated population or subpopulation of genetically engineered immune cell comprises iPSC-derived proT or T like cells. In one embodiment, the isolated population or subpopulation of genetically engineered immune cell comprises iPSC-derived proNK or NK like cells. In one embodiment, the isolated population or subpopulation of genetically engineered immune cell comprises iPSC-derived immune regulatory cells or myeloid derived suppressor cells (MDSCs). In some embodiments, the iPSC-derived genetically engineered immune cells are further modulated ex vivo for improved therapeutic potential. In one embodiment, an isolated population or subpopulation of genetically engineered immune cells that have been derived from iPSC comprises an increased number or ratio of naïve T cells, stem cell memory T cells, and/or central memory T cells. In one embodiment, the isolated population or subpopulation of genetically engineered immune cell that have been derived from iPSC comprises an increased number or ratio of type I NKT cells. In another embodiment, the isolated population or subpopulation of genetically engineered immune cell that have been derived from iPSC comprises an increased number or ratio of adaptive NK cells. In some embodiments, the isolated population or subpopulation of genetically engineered CD34 cells, HSC cells, T cells, NK cells, or myeloid derived suppressor cells derived from iPSC are allogeneic. In some other embodiments, the isolated population or subpopulation of genetically engineered CD34 cells, HSC cells, T cells, NK cells, or MDSC derived from iPSC are autogenic.

In some embodiments, the iPSC for differentiation comprises genetic imprints conveying desirable therapeutic attributes in effector cells, which genetic imprints are retained and functional in the differentiated hematopoietic cells derived from said iPSC.

In some embodiments, the genetic imprints of the pluripotent stem cells comprise (i) one or more genetically modified modalities obtained through genomic insertion, deletion or substitution in the genome of the pluripotent cells during or after reprogramming a non-pluripotent cell to an iPSC; or (ii) one or more retainable therapeutic attributes of a source specific immune cell that is donor-, disease-, or treatment response-specific, and wherein the pluripotent cells are reprogrammed from the source specific immune cell, wherein the iPSC retains the source therapeutic attributes, which are also comprised in the iPSC-derived hematopoietic lineage cells.

In some embodiments, the genetically modified modalities comprise one or more of: safety switch proteins, targeting modalities, receptors, signaling molecules, transcription factors, pharmaceutically active proteins and peptides, drug target candidates; or proteins promoting engrafiment, trafficking, homing, viability, self-renewal, persistence, immune response regulation and modulation, and/or survival of the iPSCs or derivative cells thereof. In some other embodiments, the genetically modified modalities comprise one or more of (i) deletion, disruption, or reduced expression of B2M, TAP1, TAP2, Tapasin, NLRC5, PD1, LAG3, TIM3, RFXANK, CIITA, RFX5, or RFXAP, and any gene in the chromosome 6p21 region; (ii) introduction of HLA-E, HLA-G, CD16, 4-1BBL, CD3, CD4, CD8, CD47, CD113, CD131, CD137, CD80, PDL1, A2AR, CAR, TCR, Fc receptor, or surface triggering receptors for coupling with bi- or multi-specific or universal engagers.

In still some other embodiments, the hematopoietic lineage cells comprise the therapeutic attributes of the source specific immune cell relating to one or more of (i) antigen targeting receptor expression; (ii) HLA presentation or lack thereof, (iii) resistance to tumor microenvironment; (iv) induction of bystander immune cells and immune modulations; (v) improved on-target specificity with reduced off-tumor effect; (vi) resistance to treatment such as chemotherapy; and (vii) improved homing, persistence, and cytotoxicity.

In some embodiments, the iPSC and its derivative hematopoietic cells comprise one or more of B2M null or low, HLA-E/G, PDL1, A2AR, CD47, LAG3 null or low, TIM3 null or low, TAP1 null or low, TAP2 null or low, Tapasin null or low, NLRC5 null or low, PD1 null or low, RFKANK null or low, CIITA null or low, RFX5 null or low and RFXAP null or low. These cells with modified HLA class I and/or II have increased resistance to immune detection, and therefore present improved in vivo persistence. Moreover, such cells can avoid the need for HLA matching in adoptive cell therapy and thus provide a source of universal, off-the-shelf therapeutic regimen.

In some embodiments, the iPSC and its derivative hematopoietic cells comprise one or more of CD16 or its variants including hnCD16 (high-affinity non-cleavable CD16), HLA-E, HLA-G, 4-1BBL, CD3, CD4, CD8, CD47, CD113, CD131, CD137, CD80, PDL1, A2AR, CAR, TCR, engagers, or surface triggering receptors for engagers. Such cells have improved immune effector ability.

In some embodiments, the iPSC and its derivative hematopoietic cells are antigen specific.

A variety of diseases may be ameliorated by introducing the immune cells according to embodiments of the invention to a subject suitable for adoptive cell therapy. Examples of diseases including various autoimmune disorders, including but not limited to, alopecia areata, autoimmune hemolytic anemia, autoimmune hepatitis, dermatomyositis, diabetes (type 1), some forms of juvenile idiopathic arthritis, glomerulonephritis, Graves' disease, Guillain-Barré syndrome, idiopathic thrombocytopenic purpura, myasthenia gravis, some forms of myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus, erythematosus, some forms of thyroiditis, some forms of uveitis, vitiligo, granulomatosis with polyangiitis (Wegener's); hematological malignancies, including but not limited to, acute and chronic leukemias, lymphomas, multiple myeloma and myelodysplastic syndromes; solid tumors, including but not limited to, tumor of the brain, prostate, breast, lung, colon, uterus, skin, liver, bone, pancreas, ovary, testes, bladder, kidney, head, neck, stomach, cervix, rectum, larynx, or esophagus; and infections, including but not limited to, HIV- (human immunodeficiency virus), RSV- (Respiratory Syncytial Virus), EBV- (Epstein-Barr virus), CMV- (cytomegalovirus), adenovirus- and BK polyomavirus-associated disorders.

According to some embodiments, the present invention further provides compositions for therapeutic use comprising the pluripotent cell-derived hematopoietic lineage cells made by the methods and composition disclosed herein, wherein the pharmaceutical compositions further comprise a pharmaceutically acceptable medium. In one embodiment, the composition for therapeutic use comprises the pluripotent cell-derived T cells made by the methods and composition disclosed herein. In one embodiment, the composition for therapeutic use comprises the pluripotent cell-derived NK cells made by the methods and composition disclosed herein. In one embodiment, the composition for therapeutic use comprises the pluripotent cell-derived CD34⁺ HE cells made by the methods and composition disclosed herein. In one embodiment, the composition for therapeutic use comprises the pluripotent cell-derived HSCs made by the methods and composition disclosed herein. In one embodiment, the composition for therapeutic use comprises the pluripotent cell-derived MDSC made by the methods and composition disclosed herein.

Additionally, the present invention provides, in some embodiments, therapeutic use of the above therapeutic compositions by introducing the composition to a subject suitable for adoptive cell therapy, wherein the subject has an autoimmune disorder; a hematological malignancy; a solid tumor; or an infection associated with HIV, RSV, EBV, CMV, adenovirus, or BK polyomavirus.

The isolated pluripotent stem cell derived hematopoietic lineage cells can have at least about 50%, 60%, 70%, 80%, 90%, 95%, 98%, or 99% T cells, NK cells, NKT cells, proT cells, proNK cells, CD34⁺ HE cells, HSCs, B cells, myeloid-derived suppressor cells (MDSCs), regulatory macrophages, regulatory dendritic cells or mesenchymal stromal cells. In some embodiments, the isolated pluripotent stem cell derived hematopoietic lineage cells has about 95% to about 100% T cells, NK cells, NKT cells, proT cells, proNK cells, CD34⁺ HE cells, HSCs, B cells, myeloid-derived suppressor cells (MDSCs), regulatory macrophages, regulatory dendritic cells or mesenchymal stromal cells. In some embodiments, the present invention provides therapeutic compositions having purified T cells, NK cells, NKT cells, CD34⁺ HE cells, proT cells, proNK cells, HSCs, B cells, myeloid-derived suppressor cells (MDSCs), regulatory macrophages, regulatory dendritic cells, or mesenchymal stromal cells, such as a composition having an isolated population of about 95% T cells, NK cells, NKT cells, proT cells, proNK cells, CD34⁺ HE cells, HSCs, B cells, myeloid-derived suppressor cells (MDSCs), regulatory macrophages, regulatory dendritic cells or mesenchymal stromal cells to treat a subject in need of the cell therapy.

The treatment using the derived hematopoietic lineage cells of embodiments disclosed herein could be carried out upon symptom, or for relapse prevention. The therapeutic agent or composition may be administered before, during or after the onset of a disease or an injury. The treatment of ongoing disease, where the treatment stabilizes or reduces the undesirable clinical symptoms of the patient, is also of particular interest. In particular embodiments, the subject in need of a treatment has a disease, a condition, and/or an injury that can be treated, ameliorated, and/or improved in at least one associated symptom by a cell therapy. Certain embodiments contemplate that a subject in need of cell therapy, includes, but is not limited to, a candidate for bone marrow or stem cell transplantation, a subject who has received chemotherapy or irradiation therapy, a subject who has or is at risk of having a hyperproliferative disorder or a cancer, e.g., a hyperproliferative disorder or a cancer of hematopoietic system, a subject having or at risk of developing a tumor, e.g., a solid tumor, a subject who has or is at risk of having a viral infection or a disease associated with a viral infection.

The therapeutic composition comprising derived hematopoietic lineage cells as disclosed can be administered to a subject before, during, and/or after other treatments. As such the method of a combinational therapy can involve the administration or preparation of iPSC-derived immune cells before, during, and/or after the use of an additional therapeutic agent. As provided above, the one or more additional therapeutic agents comprise a peptide, a cytokine, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD). The administration of the iPSC-derived immune cells can be separated in time from the administration of an additional therapeutic agent by hours, days, or even weeks. Additionally, or alternatively, the administration can be combined with other biologically active agents or modalities such as, but not limited to, an antineoplastic agent, a non-drug therapy, such as, surgery.

In some embodiments, the additional therapeutic agent comprises an antibody, or an antibody fragment. In some embodiments, the antibody is a monoclonal antibody. In some embodiments, the antibody may be a humanized antibody, a humanized monoclonal antibody, or a chimeric antibody. In some embodiments, the antibody, or antibody fragment, specifically binds to a viral antigen. In other embodiments, the antibody, or antibody fragment, specifically binds to a tumor antigen. In some embodiments, the tumor or viral specific antigen activates the administered iPSC-derived hematopoietic lineage cells to enhance their killing ability. In some embodiments, the antibodies suitable for combinational treatment as an additional therapeutic agent to the administered iPSC-derived hematopoietic lineage cells include, but are not limited to, anti-CD20 (retuximab, veltuzumab, ofatumumab, ublituximab, ocaratuzumab, obinutuzumab), anti-Her2 (trastuzumab), anti-CD52 (alemtuzumab), anti-EGFR (cetuximab), and anti-CD38 (daratumumab, isatuximab, MOR202), and their humanized and Fc modified variants.

In some embodiments, the additional therapeutic agent comprises one or more chemotherapeutic agents or a radioactive moiety. The term “chemotherapeutic agent” refers to cytotoxic antineoplastic agents, that is, chemical agents which preferentially kill neoplastic cells or disrupt the cell cycle of rapidly-proliferating cells, or which are found to eradicate cancer stem cells, and which are used therapeutically to prevent or reduce the growth of neoplastic cells. Chemotherapeutic agents are also sometimes referred to as antineoplastic or cytotoxic drugs or agents, and are well known in the art.

In some embodiments, the chemotherapeutic agent comprises an anthracycline, an alkylating agent, an alkyl sulfonate, an aziridine, an ethylenimine, a methylmelamine, a nitrogen mustard, a nitrosourea, an antibiotic, an antimetabolite, a folic acid analog, a purine analog, a pyrimidine analog, an enzyme, a podophyllotoxin, a platinum-containing agent, an interferon, and an interleukin. Exemplary chemotherapeutic agents include, but are not limited to, alkylating agents (cyclophosphamide, mechlorethamine, mephalin, chlorambucil, heamethylmelamine, thiotepa, busulfan, carmustine, lomustine, semustine), animetabolites (methotrexate, fluorouracil, floxuridine, cytarabine, 6-mercaptopurine, thioguanine, pentostatin), vinca alkaloids (vincristine, vinblastine, vindesine), epipodophyllotoxins (etoposide, etoposide orthoquinone, and teniposide), antibiotics (daunorubicin, doxorubicin, mitoxantrone, bisanthrene, actinomycin D, plicamycin, puromycin, and gramicidine D), paclitaxel, colchicine, cytochalasin B, emetine, maytansine, and amsacrine. Additional agents include aminglutethimide, cisplatin, carboplatin, mitomycin, altretamine, cyclophosphamide, lomustine (CCNU), carmustine (BCNU), irinotecan (CPT-11), alemtuzamab, altretamine, anastrozole, L-asparaginase, azacitidine, bevacizumab, bexarotene, bleomycin, bortezomib, busulfan, calusterone, capecitabine, celecoxib, cetuximab, cladribine, clofurabine, cytarabine, dacarbazine, denileukin diftitox, diethistilbestrol, docetaxel, dromostanolone, epirubicin, erlotinib, estramustine, etoposide, ethinyl estradiol, exemestane, floxuridine, 5-flourouracil, fludarabine, flutamide, fulvestrant, gefitinib, gemcitabine, goserelin, hydroxyurea, ibritumomab, idarubicin, ifosfamide, imatinib, interferon alpha (2a, 2b), irinotecan, letrozole, leucovorin, leuprolide, levamisole, meclorethamine, megestrol, melphalin, mercaptopurine, methotrexate, methoxsalen, mitomycin C, mitotane, mitoxantrone, nandrolone, nofetumomab, oxaliplatin, paclitaxel, pamidronate, pemetrexed, pegademase, pegasparagase, pentostatin, pipobroman, plicamycin, polifeprosan, porfimer, procarbazine, quinacrine, rituximab, sargramostim, streptozocin, tamoxifen, temozolomide, teniposide, testolactone, thioguanine, thiotepa, topetecan, toremifene, tositumomab, trastuzumab, tretinoin, uracil mustard, valrubicin, vinorelbine, and zoledronate. Other suitable agents are those that are approved for human use, including those that will be approved, as chemotherapeutics or radiotherapeutics, and known in the art. Such agents can be referenced through any of a number of standard physicians' and oncologists' references (e.g., Goodman & Gilman's The Pharmacological Basis of Therapeutics, Ninth Edition, McGraw-Hill, N.Y., 1995) or through the National Cancer Institute website (fda.gov/cder/cancer/druglistfrarne.htm), both as updated from time to time.

Immunomodulatory drugs (IMiDs) such as thalidomide, lenalidomide, and pomalidomide stimulate both NK cells and T cells. As provided herein, IMiDs may be used with the iPSC-derived therapeutic immune cells for cancer treatments.

As a person of ordinary skill in the art would understand, both autologous and allogeneic hematopoietic lineage cells derived from iPSC based on the methods and composition herein can be used in cell therapies as described above. For autologous transplantation, the isolated population of derived hematopoietic lineage cells are either complete or partial HLA-match with the patient. In another embodiment, the derived hematopoietic lineage cells are not HLA-matched to the subject.

In some embodiments, the number of derived hematopoietic lineage cells in the therapeutic composition is at least 0.1×10⁵ cells, at least 1×10⁵ cells, at least 5×10⁵ cells, at least 1×10⁶ cells, at least 5×10⁶ cells, at least 1×10⁷ cells, at least 5×10⁷ cells, at least 1×10⁸ cells, at least 5×10⁸ cells, at least 1×10⁹ cells, or at least 5×10⁹ cells, per dose. In some embodiments, the number of derived hematopoietic lineage cells in the therapeutic composition is about 0.1×10⁵ cells to about 1×10⁶ cells, per dose; about 0.5×10⁶ cells to about 1×10⁷ cells, per dose; about 0.5×10⁷ cells to about 1×10⁸ cells, per dose; about 0.5×10⁸ cells to about 1×10⁹ cells, per dose; about 1×10⁹ cells to about 5×10⁹ cells, per dose; about 0.5×10⁹ cells to about 8×10⁹ cells, per dose; about 3×10⁹ cells to about 3×10¹⁰ cells, per dose, or any range in-between. Generally, 1×10⁸ cells/dose translates to 1.67×10⁶ cells/kg for a 60 kg patient.

In one embodiment, the number of derived hematopoietic lineage cells in the therapeutic composition is the number of immune cells in a partial or single cord of blood, or is at least 0.1×10⁵ cells/kg of bodyweight, at least 0.5×10⁵ cells/kg of bodyweight, at least 1×10⁵ cells/kg of bodyweight, at least 5×10⁵ cells/kg of bodyweight, at least 10×10⁵ cells/kg of bodyweight, at least 0.75×10⁶ cells/kg of bodyweight, at least 1.25×10⁶ cells/kg of bodyweight, at least 1.5×10⁶ cells/kg of bodyweight, at least 1.75×10⁶ cells/kg of bodyweight, at least 2×10⁶ cells/kg of bodyweight, at least 2.5×10⁶ cells/kg of bodyweight, at least 3×10⁶ cells/kg of bodyweight, at least 4×10⁶ cells/kg of bodyweight, at least 5×10⁶ cells/kg of bodyweight, at least 10×10⁶ cells/kg of bodyweight, at least 15×10⁶ cells/kg of bodyweight, at least 20×10⁶ cells/kg of bodyweight, at least 25×10⁶ cells/kg of bodyweight, at least 30×10⁶ cells/kg of bodyweight, 1×10⁸ cells/kg of bodyweight, 5×10⁸ cells/kg of bodyweight, or 1×10⁹ cells/kg of bodyweight.

In one embodiment, a dose of derived hematopoietic lineage cells is delivered to a subject. In one illustrative embodiment, the effective amount of cells provided to a subject is at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, or at least 10×10⁶ cells/kg, or more cells/kg, including all intervening doses of cells.

In another illustrative embodiment, the effective amount of cells provided to a subject is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, or about 10×10⁶ cells/kg, or more cells/kg, including all intervening doses of cells.

In another illustrative embodiment, the effective amount of cells provided to a subject is from about 2×10⁶ cells/kg to about 10×10⁶ cells/kg, about 3×10⁶ cells/kg to about 10×10⁶ cells/kg, about 4×10⁶ cells/kg to about 10×10⁶ cells/kg, about 5×10⁶ cells/kg to about 10×10⁶ cells/kg, 2×10⁶ cells/kg to about 6×10⁶ cells/kg, 2×10⁶ cells/kg to about 7×10⁶ cells/kg, 2×10⁶ cells/kg to about 8×10⁶ cells/kg, 3×10⁶ cells/kg to about 6×10⁶ cells/kg, 3×10⁶ cells/kg to about 7×10⁶ cells/kg, 3×10⁶ cells/kg to about 8×10⁶ cells/kg, 4×10⁶ cells/kg to about 6×10⁶ cells/kg, 4×10⁶ cells/kg to about 7×10⁶ cells/kg, 4×10⁶ cells/kg to about 8×10⁶ cells/kg, 5×10⁶ cells/kg to about 6×10⁶ cells/kg, 5×10⁶ cells/kg to about 7×10⁶ cells/kg, 5×10⁶ cells/kg to about 8×10⁶ cells/kg, or 6×10⁶ cells/kg to about 8×10⁶ cells/kg, including all intervening doses of cells.

Some variation in dosage will necessarily occur depending on the condition of the subject being treated. The person responsible for administration will, in any event, determine the appropriate dose for the individual subject.

In some embodiments, the therapeutic use of derived hematopoietic lineage cells is a single-dose treatment. In some embodiments, the therapeutic use of derived hematopoietic lineage cells is a multi-dose treatment. In some embodiments, the multi-dose treatment is one dose every day, every 3 days, every 7 days, every 10 days, every 15 days, every 20 days, every 25 days, every 30 days, every 35 days, every 40 days, every 45 days, or every 50 days, or any number of days in-between.

The compositions comprising a population of derived hematopoietic lineage cells according to embodiments of the invention can be sterile, and can be suitable and ready for administration (i.e., can be administered without any further processing) to human patients. A cell-based composition that is ready for administration means that the composition does not require any further treatment or manipulations prior to transplant or administration to a subject. In other embodiments, the invention provides an isolated population of derived hematopoietic lineage cells that are expanded and/or modulated prior to administration with one or more agents. For derived hematopoietic lineage cells that genetically engineered to express recombinant TCR or CAR, the cells can be activated and expanded using methods as described, for example, in U.S. Pat. No. 6,352,694, the disclosure of which is incorporated herein by reference.

In certain embodiments, the primary stimulatory signal and the co-stimulatory signal for the derived hematopoietic lineage cells can be provided by different protocols. For example, the agents providing each signal can be in solution or coupled to a surface. When coupled to a surface, the agents can be coupled to the same surface (i.e., in “cis” formation) or to separate surfaces (i.e., in “trans” formation). Alternatively, one agent can be coupled to a surface and the other agent in solution. In one embodiment, the agent providing the co-stimulatory signal can be bound to a cell surface and the agent providing the primary activation signal is in solution or coupled to a surface. In certain embodiments, both agents can be in solution. In another embodiment, the agents can be in soluble form, and then cross-linked to a surface, such as a cell expressing Fc receptors or an antibody or other binding agent which will bind to the agents such as disclosed in U.S. Application Pub. Nos. 2004/0101519 and 2006/0034810, the disclosures of which are hereby incorporated by reference, for artificial antigen presenting cells (aAPCs) that are contemplated for use in activating and expanding T lymphocytes in embodiments of the present invention.

The therapeutic compositions suitable for administration to a patient can include one or more pharmaceutically acceptable carriers (additives) and/or diluents (e.g., pharmaceutically acceptable medium, for example, cell culture medium), or other pharmaceutically acceptable components. Pharmaceutically acceptable carriers and/or diluents are determined in part by the particular composition being administered, as well as by the particular method used to administer the therapeutic composition. Accordingly, there is a wide variety of suitable formulations of therapeutic compositions according to embodiments of the present invention (see, e.g., Remington's Pharmaceutical Sciences, 17^{th} ed. 1985, the disclosure of which is hereby incorporated by reference in its entirety).

In particular embodiments, therapeutic cell compositions having an isolated population of iPSC-derived hematopoietic lineage cells also have a pharmaceutically acceptable cell culture medium, or pharmaceutically acceptable carriers and/or diluents. A therapeutic composition comprising a population of iPSC-derived hematopoietic lineage cells as disclosed herein can be administered separately by intravenous, intraperitoneal, enteral, or tracheal administration methods or in combination with other suitable compounds to effect the desired treatment goals.

These pharmaceutically acceptable carriers and/or diluents can be present in amounts sufficient to maintain a pH of the therapeutic composition of between about 3 and about 10. As such, the buffering agent can be as much as about 5% on a weight-to-weight basis of the total composition. Electrolytes such as, but not limited to, sodium chloride and potassium chloride can also be included in the therapeutic composition. In one aspect, the pH of the therapeutic composition is in the range from about 4 to about 10. Alternatively, the pH of the therapeutic composition is in the range from about 5 to about 9, from about 6 to about 9, or from about 6.5 to about 8. In another embodiment, the therapeutic composition includes a buffer having a pH in one of said pH ranges. In another embodiment, the therapeutic composition has a pH of about 7. Alternatively, the therapeutic composition has a pH in a range from about 6.8 to about 7.4. In still another embodiment, the therapeutic composition has a pH of about 7.4.

The invention also provides, in part, the use of a pharmaceutically acceptable cell culture medium in particular compositions and/or cultures according to embodiments of the present invention. Such compositions are suitable for administration to human subjects. Generally speaking, any medium that supports the maintenance, growth, and/or health of the iPSC-derived immune cells in accordance with embodiments of the invention are suitable for use as a pharmaceutical cell culture medium. In particular embodiments, the pharmaceutically acceptable cell culture medium is a serum-free, and/or feeder-free medium. In various embodiments, the serum-free medium is animal-free, and can optionally be protein-free. Optionally, the medium can contain biopharmaceutically acceptable recombinant proteins. Animal-free medium refers to medium wherein the components are derived from non-animal sources. Recombinant proteins replace native animal proteins in animal-free medium and the nutrients are obtained from synthetic, plant or microbial sources. Protein-free medium, in contrast, is defined as substantially free of protein. One having ordinary skill in the art would appreciate that the above examples of media are illustrative and in no way limit the formulation of media suitable for use in embodiments of the present invention.

### EXAMPLES

The following examples are offered by way of illustration and not by way of limitation.

### Example 1—Material and Methods

Single cell dissociation All reprogramming cultures were switched to FMM or FMM2 on around day 14 post transfection. Once in FMM/FMM2 all reprogramming cultures were maintained and dissociated using Accutase. Single cells were then passaged on either Matrigel or Vitronectin coated surface. The single cell dissociated cells were then expanded in FMM or FMM2 and maintained until flow cytometry sorting.

Flow Cytometry Analysis and Sorting Single cell dissociated reprogramming pools were resuspended in chilled staining buffer. Conjugated primary antibodies, including SSEA4-FITC, TRA181-Alexa Fluor-647 and CD30-PE (BD Biosciences), were added to the cell solution and incubated on ice for 15 min. All antibodies were used at 7-10 μL in 100 μL staining buffer per million cells. The resuspended dissociated single cells in staining buffer were spun down and resuspended in staining buffer now containing a ROCK inhibitor and maintained on ice for flow cytometry sorting. Flow cytometry sorting was performed on FACS Aria II (BD Biosciences). The sorted cells were directly ejected into 96-well plates at concentrations of 3 and 9 events per well. Each well was prefilled with FMM2. Upon completion of the sort, 96-well plates were incubated for colony formation and expansion. Seven to ten days post sort, the cells were passaged. Subsequent passages in FMM2 were done routinely upon 75-90% confluency. Flow cytometry analysis was performed on Guava EasyCyte 8 HT (Millipore) and analyzed using FCS Express 4 (De Novo Software).

Testing Presence of Transgenes Genomic DNA was isolated using QIAamp® DNA Mini Kit and Proteinase K digestion (Qiagen). 100 ng of the genomic DNA was amplified using primer sets specific to transgenes including the reprogramming factors and EBNA1 using Taq PCR Master Mix Kit (Qiagen). The PCR reactions were run for 35 cycles as follows: 94° C. for 30 sec (denaturation), 60-64° C. for 30 sec (annealing) and 72° C. for 1 min (extension). Genomic DNA from fibroblasts, T cells, and hiPSCs generated using lentiviral methods were used as negative controls. DNA of the episomal constructs was used as positive control.

Karyotype Analysis Cytogenetic analysis was performed on G-banded metaphase cells by WiCell Research Institute (Madison, WI). Each karyotype analysis includes a minimum count of 20 spreads with analyses expanded to 40 spread counts when nonclonal aberrations are identified in the first 20.

Statistical Analysis At least three independent experiments were performed. Values are reported as mean+SEM. Statistical analysis was done with ANOVA with p<0.05 considered significant.

Culture Media Conventional hESC culture contains DMEM/F12 culture medium supplemented with 20% KnockOut serum replacement, 0.1 mM (or 1% v/v) non-essential amino acids, 1-2 mM L-glutamine, 0.1 mM ε-mercaptoethanol and 10-100 ng/ml bFGF). In comparison, the multistage culture media additionally comprises one or more of an HDAC inhibitor, a ROCK inhibitor, a GSK3 inhibitor/WNT activator, a MEK inhibitor and a TGFβ inhibitor. This stage-specific culture platform also supports feeder-free reprogramming and maintenance.

In certain applications, in addition to the ingredients for conventional culture, the reprogramming medium (e.g., FRM) contains SMC4: a combination of ROCK inhibitor, GSK3 inhibitor/WNT activator, MEK inhibitor and TGFβ inhibitor. For the STTR2 reprogramming system, the enhanced reprogramming medium (e.g., FRM2) does not comprise the TGFβ inhibitor until the cell loses its somatic cell identity (for example, the loss of cell type specific gene expression) at around day 6-8, and comprises an HDAC inhibitor optionally added later than the ROCKi, MEKi and GSK3i. In certain applications, the maintenance medium (FMM) contains SMC3: a combination of ROCK inhibitor, GSK3 inhibitor, and MEK inhibitor. For the STTR2 reprogramming, the enhanced maintenance medium (FMM2) contains a member of the transforming growth factor beta superfamily (examples of which include, but are not limited to, Activin A, TGFβ and Nodal), optionally with the concentration of one or both of GSK3 inhibitor and MEK inhibitor reduced by 30/6-60% in comparison to that in FRM2. Nodal is a secretory protein that belongs to the TGFb superfamily, and is encoded by the NODAL gene located on chromosome 10q22.1 in human. TGFβ is a multifunctional cytokine belonging to the the TGFb superfamily. Activin A is a TGFβ superfamily cytokine closely related to TGFβ.

### Example 2—Enhancing Long-Term Stability and Preservation Using FMM2

Long-term preservation of induced pluripotent stem cells (iPSCs), especially in feeder cell-free conditions, is affected by various stressors in cell manipulation processes including, but not limited to, single cell dissociation and sorting, clonal expansion, freeze-thaw cycles, vector transfection and electroporation and genomic editing, which result in genomic instability of the cells, as may be detected by G-banded karyotyping, droplet digital PCR, including various karyotype abnormalities. Among chromosomal deletions, duplications, translocations, or inversions, trisomy of certain chromosomes has been observed more frequently in iPSCs reprogrammed from T cells (TiPSC) than other somatic cell types such as fibroblasts. In addition, these stressors compromise pluripotency, viability and the differentiation potential of the obtained pluripotent cells, which are often banked with cryopreservation over an extended period of time.

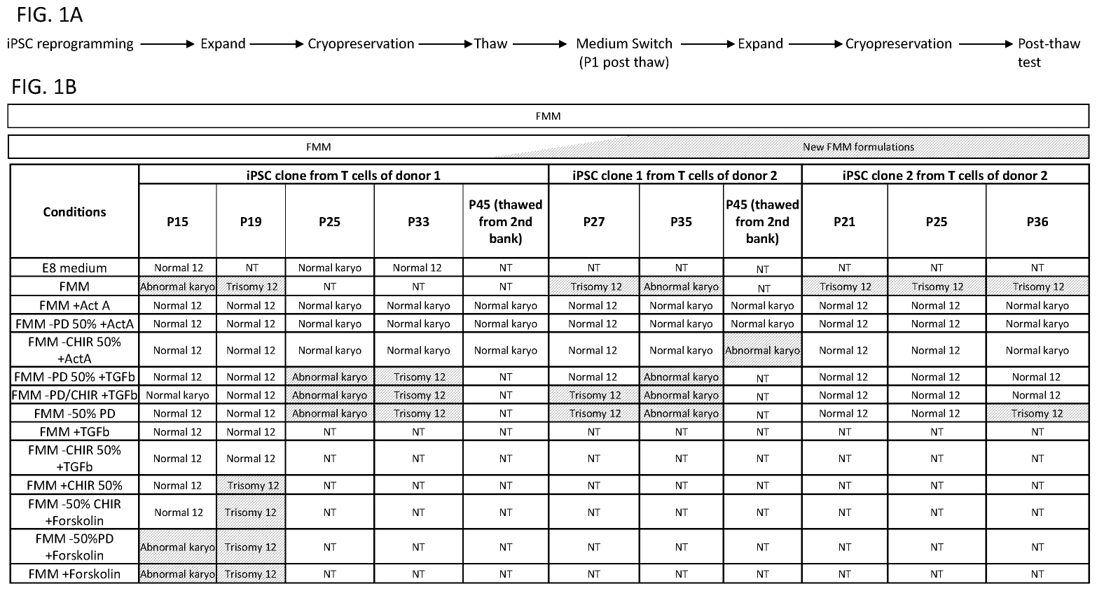

Previously developed FMM (Fate Maintenance Medium) has achieved satisfying long-term stability of iPSCs reprogrammed from various somatic cells that are not T cells. In addition to some base ingredients, FMM comprises small molecules such as a ROCK inhibitor, a WNT activator, and a MEK inhibitor. To enhance long-term stability and preservation and reduce the frequency of karyotype abnormalities of iPSCs reprogrammed from all cell sources, and especially from T cells, various modifications of FMM were carried out as disclosed and exemplified herein in FIG. 1A, and the modified and enhanced iPSC maintenance medium is referred to as FMM2 from time to time in this application. T cell reprogramming was initiated using non-integrating STTR plasmids and FRM (Fate Reprogramming Medium) or the modified and enhanced FRM, referred to as FRM2, and the resulting cells were either transfected with CRISPR ribonucleoprotein (RNP) complexes that mediate locus-specific targeted-insertion or deletion (engineered) or proceeded directly to single cell sorting (non-engineered) to generate single cell sorted, engineered or non-engineered, iPSC clones. To test the effect of FMM2 impact to long-term iPSC preservation, and especially to iPSC banks that undergo cycles of freeze-thaw, the single cell sorted iPSC clones were expanded in FMM and then cryopreserved (1^{st} bank) in FMM as well. iPSC clones from 1^{st} bank were thawed, expanded in FMM or FMM2, and cryopreserved in respective FMM or FMM2 to make a secondary bank (2^{nd} bank). The FMM modification is mainly to the small molecule composition of FMM, and the tested modification includes supplementation of at least one of Activin A (Act A), TGFβ, and Forskolin; in combination with or without concentration reduction of MEK inhibitor (MEKi) and/or GSK3 inhibitor (GSKi) by about 40-60% as compared to FMM. Here, 50% concentration reduction in MEKi or GSK3i was used for the purpose of illustration.

2^{nd} bank post-thaw tests were performed to test long-term stability of iPSC clones with different medium treatment. Genomic stability was examined by two independent methods: chromosome 12 copy number determination by ddPCR and whole genome stability as determined by G-banded karyotyping analysis at the indicated passage. G-banded karyotyping revealed microscopic genomic abnormalities (>5 Mb) including inversions, duplications/deletions, balanced and unbalanced translocations, and aneuploidies with sensitivity of >10% mosaicism.

As shown in FIG. 1B, one iPSC clone derived from T cell donor 1 and two iPSC clones from T cell donor 2 were tested as described above. Karyotype results were indicated as “normal karyo” (i.e., 46, XY or 46, XX) and “abnormal karyo”. Clones having abnormal karyotype was further analyzed using ddPCR to determine the presence of trisomy 12. ddPCR results were indicated as “normal 12” (i.e., copy number <2.3) and “trisomy 12” (i.e., copy number >2.3). Irrespective of the different extents of the benefits from various FMM modifications, and given consideration of donor and clone disparity, it was found that supplementing FMM with members of the TGFβ family (such as Activin A, TGFβ, or Nodal) with or without the combination of a concentration reduction in MEK and GSK3 inhibitors in FMM improves and enhances long-term stability of iPSCs, including TiPSCs, as indicated by a reduction of chromosome 12 trisomy and maintenance of normal karyotype over extended passages and through multiple freeze-thaw cycles (and possibly with expansion, genomic engineering and sorting in-between thawing and freezing), as compared to TiPSCs cultured in FMM without the benefit of exposure to the above described modification to FMM.

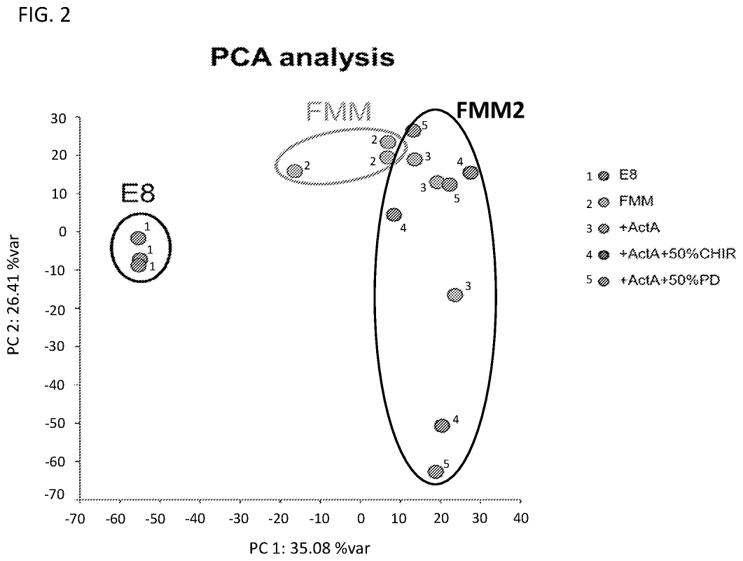

E8 is a commercial medium for iPSC maintenance and preservation, and is known to promote primed pluripotency in iPSCs. TiPSCs generated using the reprogramming platform as described were each maintained in E8, FMM or FMM2 for more than 10 passages, and were harvested for gene expression profiling and Principal Component Analysis (PCA). As shown in FIG. 2, TiPSC clones maintained in the E8, FMM or FMM2 formed three distinctive clusters with each correlating to the medium to which the cells were exposed.

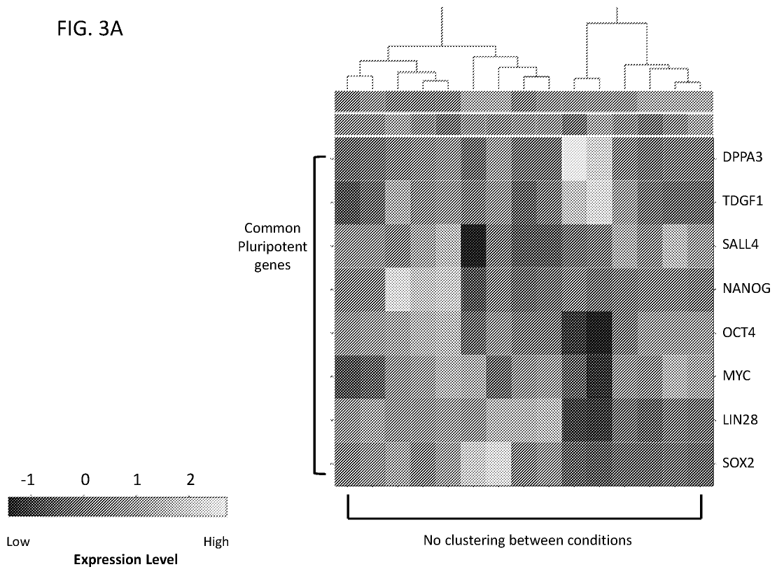

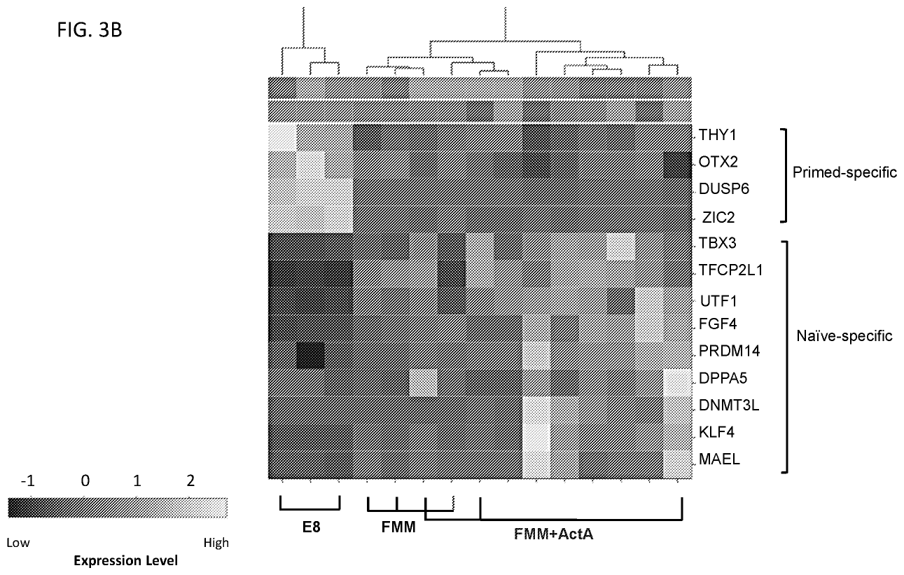

The E8, FMM or FMM2 cultured TiPSC clones were further prepared for RNA-seq analysis of pluripotency markers. Common pluripotency markers including DPPA3, TDGF1, SALL4, NANOG, OCT4, MYC, LIN28 and SOX2 were expressed in all culture conditions tested without clustering. Notably, primed pluripotency specific genes such as THY1, OTX2, DUSP6 and ZIC2 were up-regulated in TiPSCs under the E8 condition. Clones showed low level expression of primed-specific markers and moderate level expression of naïve-specific markers, for example, TBX3, TFCP2L1, UTF1, FGF4, PRDM14, DPPA5, DNMT3L, KLF4 and MAEL. The expression of all those naïve-specific markers was further elevated in the TiPSC clones cultured in FMM2, in this example, with the Activin A addition. In addition, the FMM2 cultured iPSCs also most distinctively express very high levels of PRDM14, DPPA5, DNMT3L, KLF4 and MAEL, a group of additional naïve pluripotency specific genes that are mostly silenced in iPSC maintained in E8 or FMM conditions (see FIGS. 3A and 3B), demonstrating the capability of FMM2 in deepening the pluripotency level of iPSCs on the spectrum or continuum of pluripotency (for example, from primed to naïve, from low naïvety to higher naïvety), and its application in adapting iPSCs of a lower pluripotency level, irrespective of reprogramming process used, to a higher level on the pluripotency spectrum.

### Example 3—FMM2 Prevents Stress-Induced Genomic Abnormalities in iPSCs

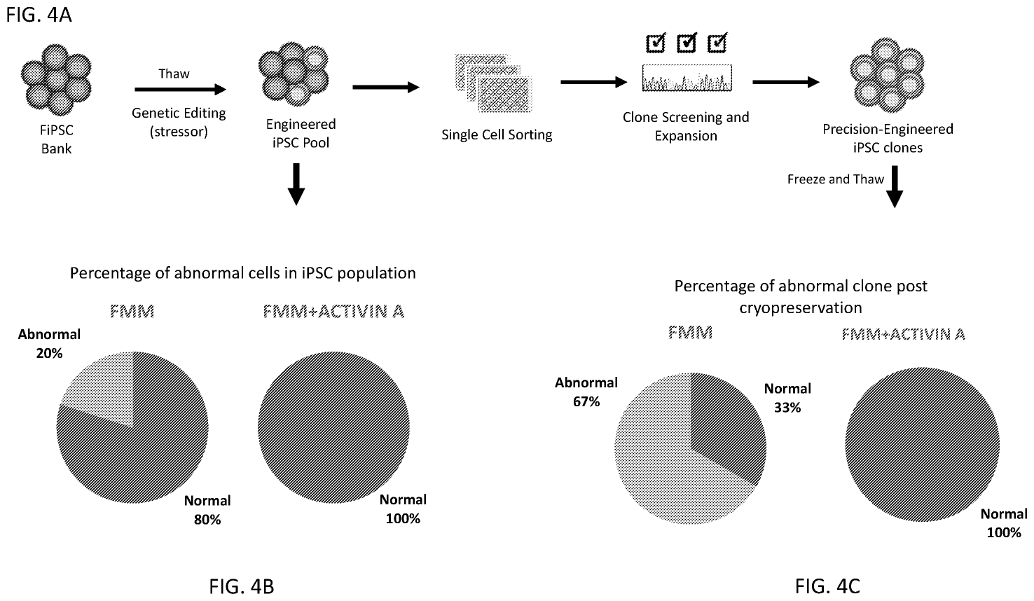

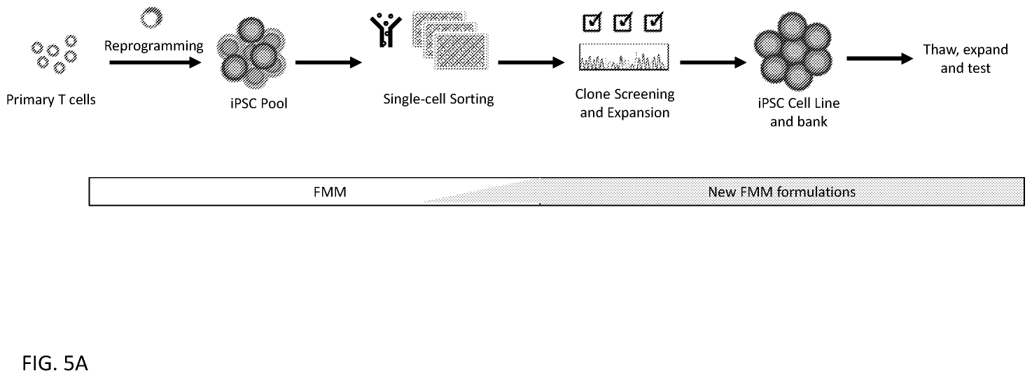

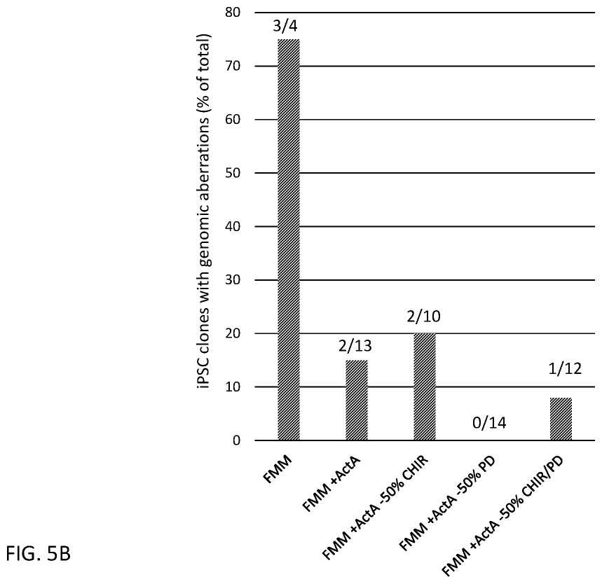

To test whether addition of a member of the TGF beta superfamily to FMM improves genomic stability after stress-inducing genetic engineering and cryopreservation of previously banked iPSCs, clonal iPSC samples of the above-described 1^{st} bank were thawed, engineered, sorted, expanded and again cryopreserved separately in FMM or in FMM supplemented with about 10-30 ng/mL of Activin A to generate a secondary bank, as shown in FIG. 4A. Engineering was performed by electroporating iPSCs with CRISPR RNP complexes that mediate targeted insertion of desired transgenes into a specified genomic locus, as described above.

Samples of transfected populations (engineered iPSC pool, and the iPSCs were from the 1^{st} cryopreservation bank) were harvested for karyotyping analysis, while the rest of the cells were used for single cell sorting to generate clonal engineered iPSCs. Karyotyping analysis was performed post-transfection on engineered iPSC pool and on sorted and expanded clonal iPSC population cultured in FMM or FMM+Activin A. As shown in FIG. 4B, for cell populations manipulated by genetic engineering, only 80% of the engineered cell population cultured in FMM presented a normal karyotype (46+XY), whereas 100% of engineered cells cultured in FMM2 presented a normal karyotype.

Of the transfected populations that were further subject to single cell sorting, individual clones were screened for defined attributes including successful and precise engineering. Accepted clones were expanded and cryopreserved as individual clonal populations (FIG. 4A) to generate a secondary (2^{nd}) iPSC cryopreservation bank. The iPSCs were then taken from the 2^{nd} bank and thawed, and maintained in FMM or FMM2 medium. The results of karyotyping analysis and copy number variation analysis by SNP microarray assay performed post-cryopreservation/thawing on iPSC clonal population of a 2^{nd} cryopreservation bank are shown in FIG. 4C: only one out of three clones (33%) generated in FMM presented a normal karyotype (46+XY), whereas 100% of the clones generated in FMM2 presented a normal karyotype (46+XY). All clones generated in FMM2 were shown to have no reportable copy number changes and no reportable regions of loss/absence of heterozygosity by SNP microarray analysis despite the multiple rounds of freeze and thaw process. As such, multiple stressors in iPSC manufacturing process that involves engineering, single cell sorting, screening, expansion, and multiple cycles of freeze and thaw, have accumulative negative effects on iPSC genomic stability, which, however, could be reduced or prevented by FMM2 that comprises addition of a member of the TGFb superfamily including at least Activin A, TGFb, and Nodal.

### Example 4—FMM2 Leads to Improved Genomic Stability of iPSCs Reprogrammed from T Cells

To generate iPSCs reprogrammed from T cells (TiPSCs), primary T cells were transfected with reprogramming plasmids to produce an iPSC pool that is heterogenous in nature. Single cell sorting was performed to establish iPSC clones. As shown in FIG. 5A, the post-sorting TiPSC clones were expanded in FMM, or various forms of FMM2: FMM+ActA; FMM+ActA, with a 50% reduction in GSK3 inhibitor concentration than that was used in FMM (FMM+ActA−50% CHIR), FMM+ActA, with a 50% reduction in MEK inhibitor concentration (FMM+ActA−50% PD); or FMM+ActA, with 50% reductions in both GSK3 inhibitor and MEK inhibitor concentrations (FMM+ActA−50% CHIR/PD).

Screening criteria of fully reprogrammed TiPSC clones included morphology and pluripotent marker expression. Accepted clones were expanded and cryopreserved in each of the above indicated media. Genomic stability was first examined by determining chromosome 12 copy number by droplet digital PCR (ddPCR) pre-cryo and then selected clones were tested for whole genome stability by G-banded karyotyping analysis post-cryo. As shown in FIG. 5B, copy number determination by ddPCR and karyotype analysis revealed significant reductions in genomic aberrations using the various FMM2 formulations, as compared to FMM in the context of T cell reprogramming. Even without engineering or multiple freeze-thaw cycles, the genomic aberration rate of TiPSCs under the FMM condition is around 75%, close to that observed with FiPSCs that went through multiple freeze-thaw cycles of and additional stressful cell manipulations, reflecting the difficulty in T cell reprogramming. In contrast, the genomic aberration rates of TiPSCs under the FMM2 conditions are substantially lower: 0% under FMM+ActA−50% PD, and around 8%, 15% and 20% under FMM+ActA−50% CHIR/PD, FMM+ActA, and FMM+ActA−50% CHIR, respectively.

### Example 5—Reprogramming Using Enhanced Transient and Temporal Reprogramming System in Addition to FMM2 iPSC Maintenance

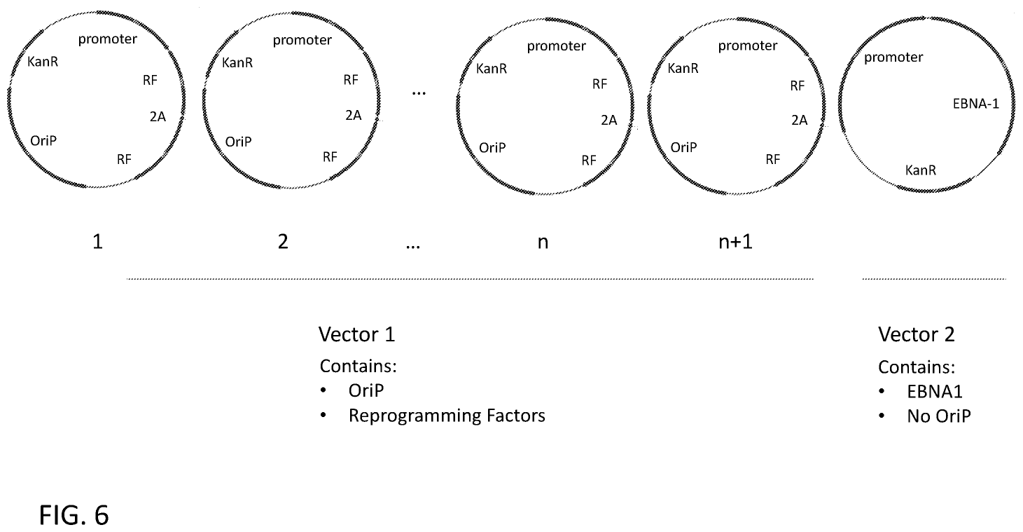

The vectors required in the transient and temporal reprogramming system (STTR) are shown in FIG. 6. Vector 1 is a plasmid vector containing an oriP, a promoter, which drives the expression of one or more operatively linked selected reprogramming factor(s) (RF). Vector 1 is also termed as oriP/RF plasmid. Where there are two or more RFs in one vector 1 plasmid, the neighboring RFs are separated by a 2A peptide or IRES. Vector 1 does not have an EBNA encoding sequence, and has shortened retention time in a host cell as a result. Depending on the total number of RFs used for reprogramming, multiple Vector 1 plasmids can be used, which collectively contain all selected RFs, in different combinations as desired. Further, using multiple Vector 1 plasmids for co-transfection is desirable where stoichiometry of the reprogramming factors is refined by controlling the relative copy number of each reprogramming factor in a combination of multiple Vector 1 plasmids. Vector 2 is a plasmid containing a promoter and an EBNA encoding sequence, which expression is driven by the promoter. More importantly, Vector 2 lacks oriP which leads to significantly reduced Vector 2 retention time in the transfected host cell population. The segregation of EBNA and oriP sequences in separate constructs ensures transient expression of transgenes and faster and earlier spontaneous loss of reprogramming vectors as cells divide, leading to footprint-free iPSCs. Vector 2 can also be replaced with EBNA mRNA or protein/peptide.

To illustrate, a series of Vector 1 constructs, and a Vector 2 construct were made as set forth in Table 3 and FIG. 6. Reprogramming factors used in this exemplary system included four Vector 1 plasmids, each containing OCT4 and YAP1, SOX2 and MYC, LIN28 and Large T antigen (LTag), and ESRRB and ZIC3, respectively. However, it should be understood that any number of Vector 1 plasmids may be used, and the order of the RFs in one Vector 1 plasmid may also vary, provided that the multiple Vector 1 plasmids collectively comprise polynucleotides encoding at least OCT4, YAP1, SOX2 and LTag.

TABLE 3
Vector Construction

| | Vector Description | Notes |
|---|---|---|
| Vector 1 | | |
| 1a | promoter -OCT4-P2A-YAP1-oriP | Does not contain EBNA; |
| 1b | promoter -SOX2-P2A-MYC-oriP | has shortened retention |
| 1c | promoter -LIN28-P2A-LTag-oriP | time in host cell as |
| 1d | promoter -ESRRB-P2A-ZIC3-oriP | a plasmid; |
| Vector 2 | promoter -EBNA-1 | Does not contain oriP; |
| | | has shortened retention |
| | | time in host cell |

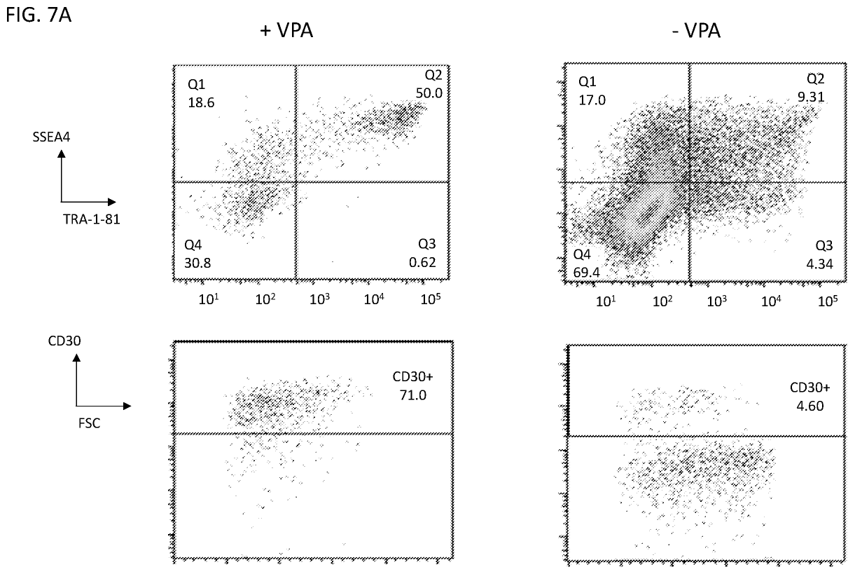

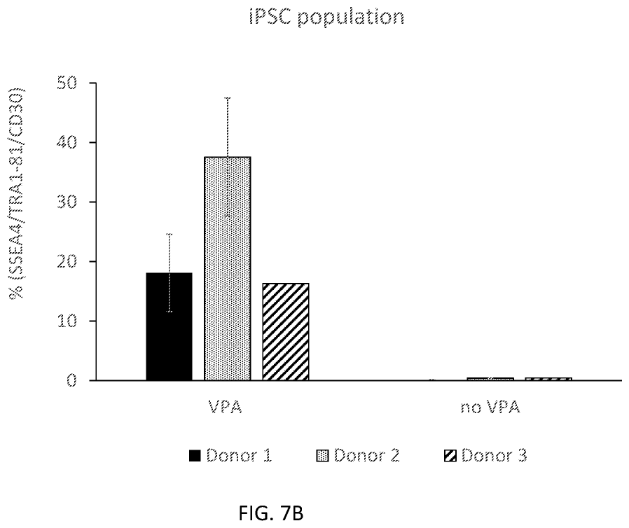

Human T cells from three different donors were transfected with the above reprogramming plasmids on day 0. To determine an whether an HDAC inhibitor improves reprogramming efficiency, or particularly, T cell reprogramming efficiency as compared to the reprogramming culture without the same treatment, around day 2-3 post transfection, the reprogramming culture was treated with valproic acid (VPA) until a heterogenous iPSC population was generated. The expression of iPSC surface markers (SSEA4, TRA-1-81, and CD30) was analyzed by flow cytometry. As shown in FIGS. 7A and 7B, it was found that VPA treatment significantly increased the percentage of SSEA4⁺TRA-1-81⁺CD30⁺ iPSC cells in the population across different T cell donors, thereby demonstrating VPA's advantage in potentiating T cell reprogramming with improved efficiency.

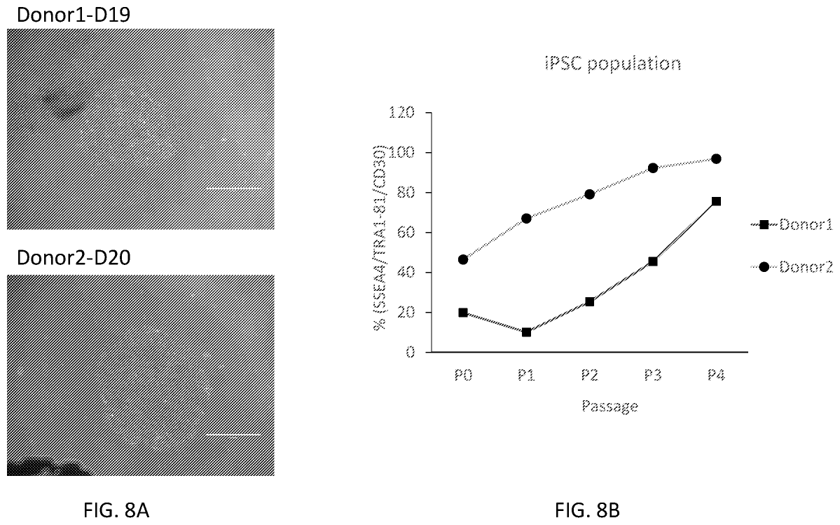

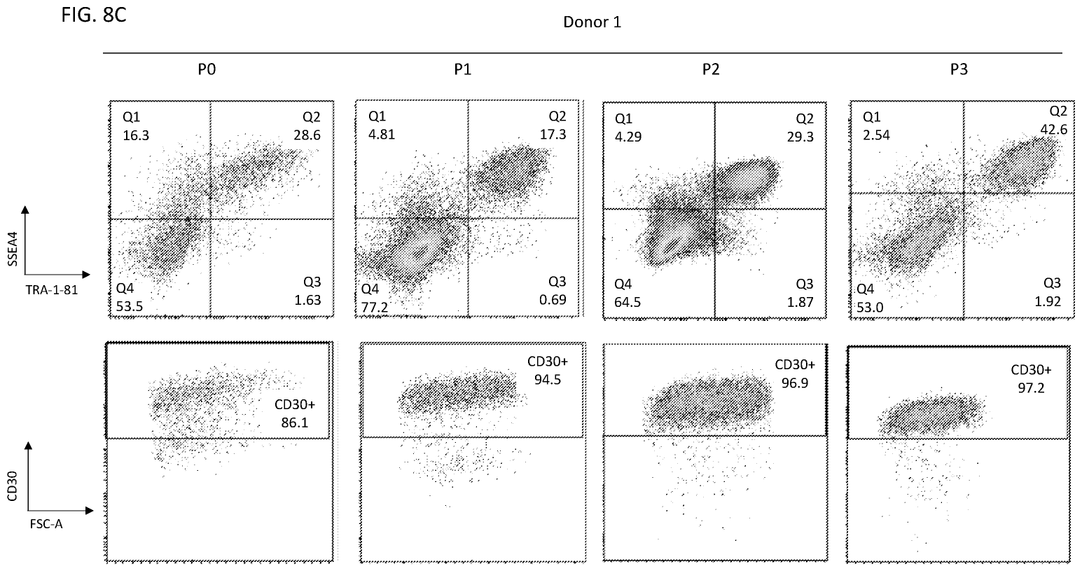

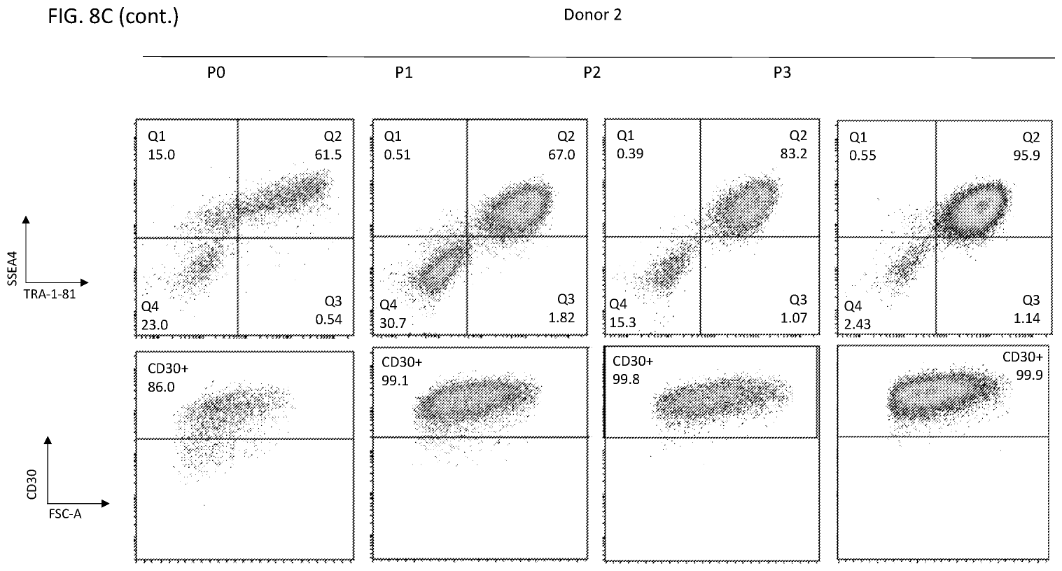

Clonal expansion of single cells dissociated from the iPSC colonies generated using the above reprogramming system (FIG. 8A) was performed to induce stress and the stability of the expanded pluripotent cultures was determined across different donors. As shown in FIG. 8B, fractions of iPSC populations increased over continuous passaging indicating stable and self-renewal pluripotent cultures derived from multiple donors. Flow cytometry analysis of the passaged reprogramming pools confirmed expression of the iPSC pluripotency markers indicating sustained pluripotency after expansion (FIG. 8C).

A previous FRM (Fate reprogramming medium) comprising a ROCK inhibitor, a WNT activator, a MEK inhibitor, and a TGFβ inhibitor has been used for somatic cell reprogramming, and is conventionally applied soon after transfection. The cells in the reprogramming system are in the presence of the FRM from around day 1 until an iPSC pool is generated, a process of 12-16 days. It was discovered that exposing the somatic cells transfected with STTR plasmids to small molecules, including VPA and TGFβ inhibitor, in a stage-specific manner could further improve the quality and the efficiency of the reprogramming process during the 12-16 day process as shown in FIG. 9, and this STTR plus FRM comprising stage-specific HDACi and TGFβi is also called “STTR2 reprogramming” in this application.

In the STTR2 method and composition, in addition to the valproic acid (VPA) treatment as described above, around the time of expression of the exogenous genes in the vector plasmids until the time when iPSC colonies appear (from around day 2-3 until day 12-16 post transfection), the exposure to a TGFβ inhibitor is delayed from around day 1 in the STTR method to around day 6-8 in the STTR2 method when the transfected somatic cells loose T cell identity until the iPSC colony formation at around day 12-16. The ROCK inhibitor, WNT activator, and the MEK inhibitor remain in the medium after reprogramming, taking the cells through single cell dissociation, single cell sorting to establish iPSC clones. Screening criteria of fully reprogrammed iPSC clones included morphology, pluripotent marker expression and clearance of reprogramming plasmids. Accepted clones were expanded and cryopreserved as a master cell bank (MCB) comprising high purity clonal iPS cells (>99%) using FMM2 as described above, i.e., with Activin A addition after sorting or after iPSC colony formation, and optionally concentration reduction of one or both of MEK inhibitor and WNT activation by around 30%-60%. Karyotyping analysis and pluripotency gene profiling were used to determine post-cryo stability of iPSC clones.

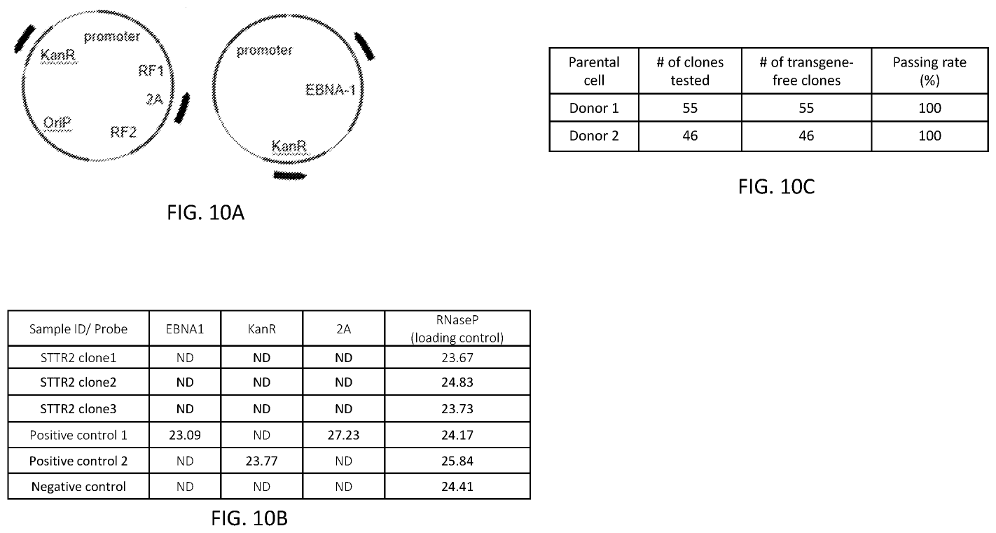

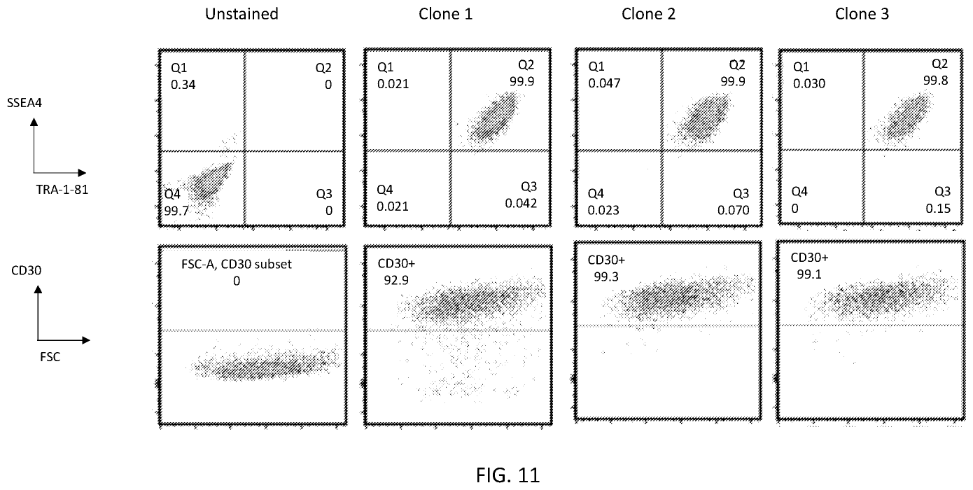

TaqMan probes (FIG. 10A; black bars) were used for detection of reprogramming vectors in iPSC clones. It was observed that reprogramming different donor T cells using the STTR2 system led to robust generation of iPSC clones that are 100% transgene-free with complete vector clearance (FIGS. 10B and 10C), indicating a higher quantity and more reliable footprint-free outcome as compared to previous systems. Further, flow cytometry analysis of the STTR2-produced iPSC clones showed homogenous expression of iPSC surface markers (SSEA4, TRA-1-81 and CD30) (FIG. 11). All iPSC clones were equally and distinctly divergent in their gene expression profiles compared to parental T cells, confirming that the STTR2 system led to the generation of high-quality pluripotent cells derived from terminally differentiated T cells, which are particularly challenging to reprogram compared to fibroblasts or keratinocytes, for example.

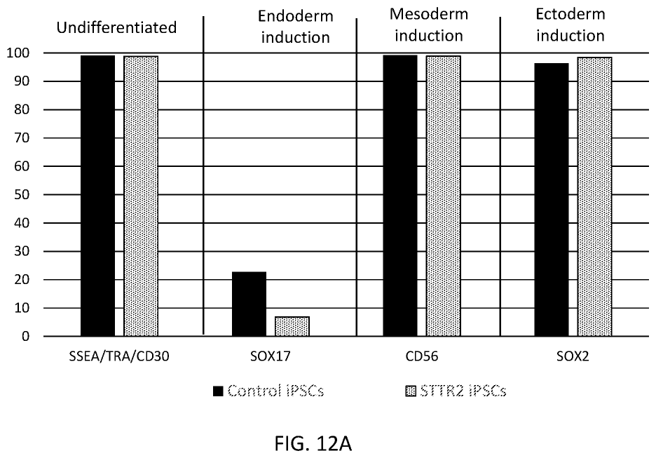

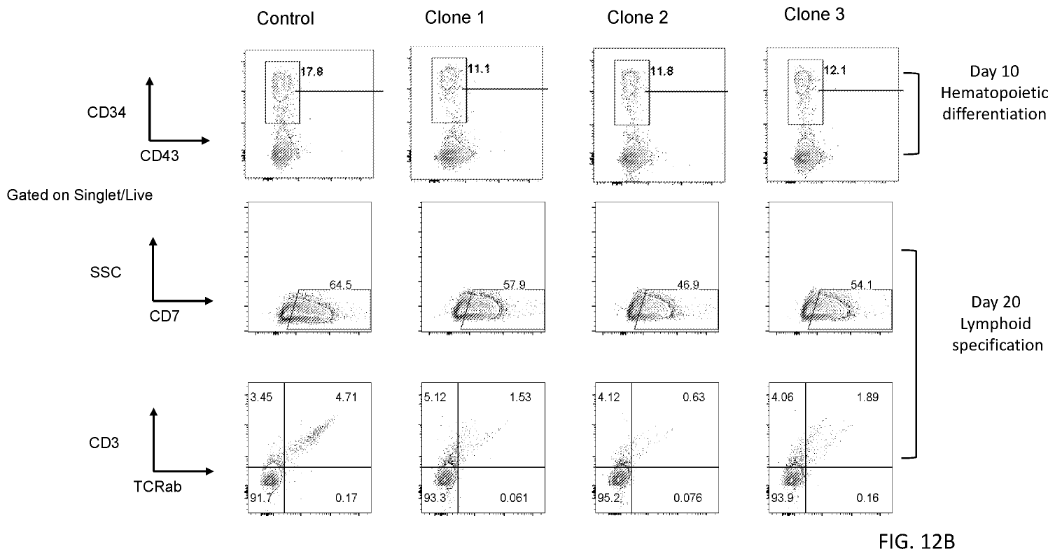

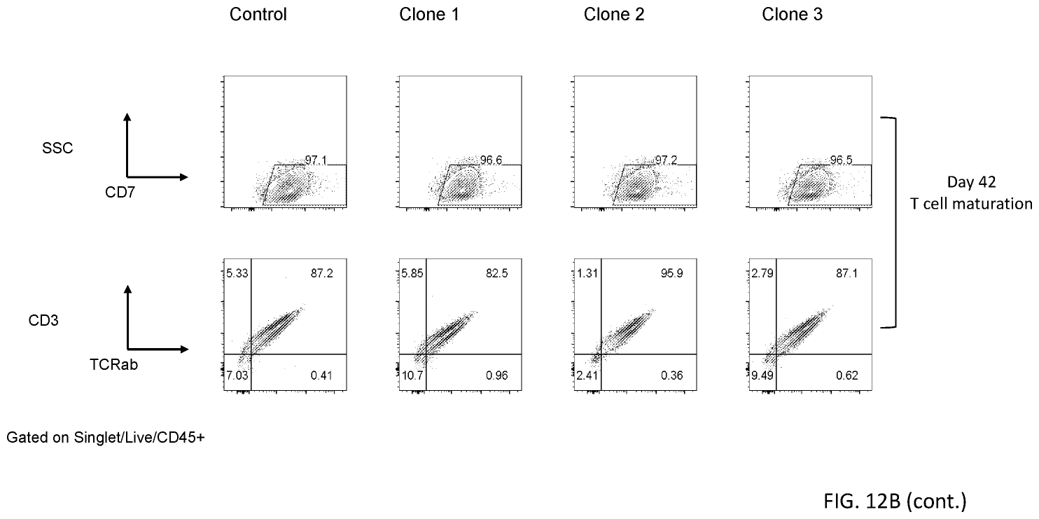

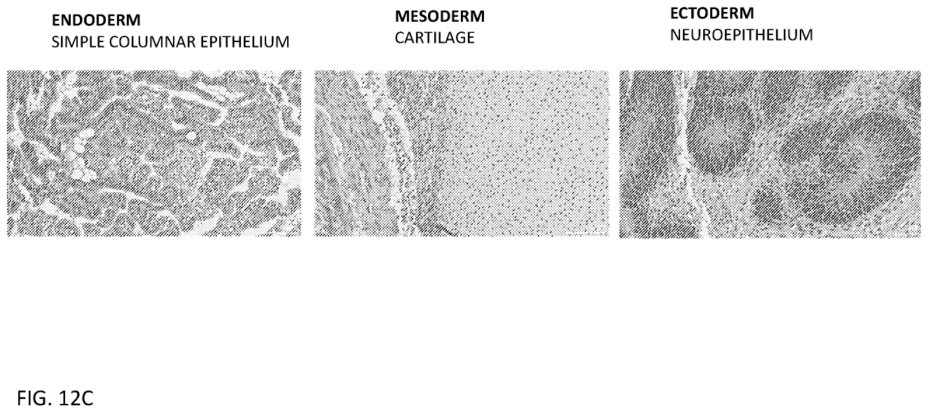

Further, iPSC clones generated by STTR2 were found to maintain a high propensity to differentiate into cell types representing all three germ layers. Pluripotency of iPSC generated by STTR2 were evaluated by testing their trilineage differentiation potential. iPSC differentiation was performed using a STEMdiff™ Trilineage Differentiation Kit (Stem Cell Technologies). One week after culturing in indicated media to induce lineage-specific differentiation, differentiated cells were harvested and expression of indicated lineage markers (pancreatic progenitor marker SOX17 for endoderm, mesenchymal marker CD56 for mesoderm, and neural progenitor marker SOX2 for ectoderm) was assessed by flow cytometry (FIG. 12A).

Pluripotency of iPSCs generated by STTR2 was evaluated by testing their ability to differentiate toward terminally differentiated cells like T lymphocytes. iPSCs were cultured in stage-specific media to induce hematopoietic specification and T cell differentiation. As shown in FIG. 12B, flow cytometry analyses at indicated timepoints demonstrated that iPSCs generated by STTR2 differentiated into mature T cells similar to control iPSCs generated using a conventional episomal system.

In a separate experiment, pluripotency of iPSCs obtained using STTR2 was evaluated by an in vivo teratoma formation assay. 0.5-2 million of iPSCs generated by STTR2 were implanted to immunodeficient NSG mice by subcutaneous injection. 6-10 weeks after injection, teratoma tissues were harvested, processed, and subjected to histology analysis including staining of paraffin-embedded tissue sections with heatoxylin and eosin. As shown in FIG. 12C, pluripotency of iPSC clones generated using the STTR2 system was confirmed as the teratoma contains tissues derived from each of the embryonic germ layers: endoderm, mesoderm, and ectoderm.

Collectively, the data show that footprint-free iPSCs can be readily generated by transiently and temporarily expressing reprogramming genes using the enhanced STTR2 system, which comprises reprogramming stage specific small molecules such as HDACi and TGFβi in addition to ROCKi, MEKi and GSK3i of FMM. The enhanced platform supports efficient and expedited generation of a substantially homogenous footprint-free iPSC population, including TiPSCs, that maintains pluripotency over extensive passaging.

### Example 6—iPSCs Obtained Using STTR2 Maintain Genome Stability Post Sequential Engineering and Produce Functional T Cells

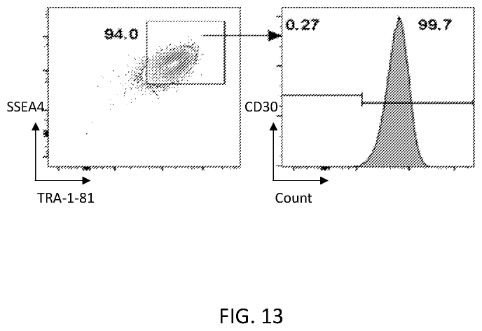

iPSC clones were thawed and expanded as described in Example 2. In this experiment, engineering was performed by electroporating iPSCs with CRISPR RNP complexes that mediate targeted insertion of a Chimeric Antigen Receptor (CAR)-expressing cassette into a specified genomic locus. The engineered population of cells was single-cell sorted and expanded for screening of desired genetic modalities. Selected clones exhibiting normal karyotypes (46, XX) at the latest passage tested (i.e., passage 10 following iPSC thawing) were expanded and cryopreserved in FMM2 and tested for genomic stability by karyotype analysis post cryopreservation. As shown in FIG. 13, flow cytometry profiles of STTR2-generated, CAR-engineered iPSC clones showed homogenous expression of iPSC surface markers (SSEA4, TRA-1-81 and CD30).

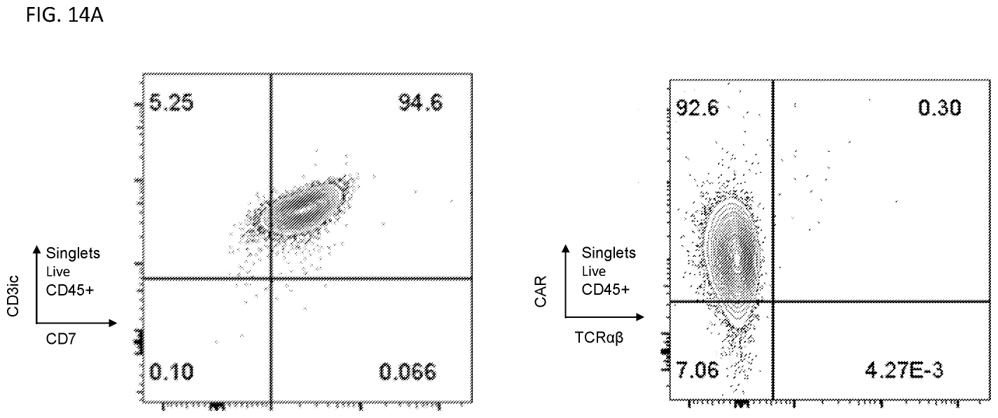

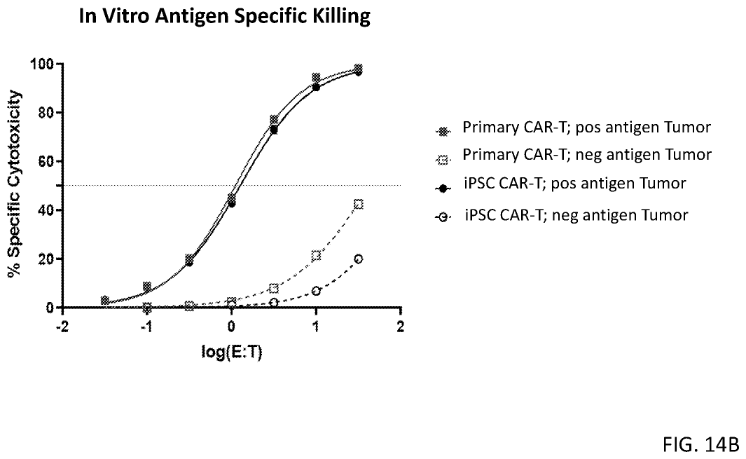

In a separate experiment, the iPSCs were cultured in stage-specific media to induce hematopoietic specification and T cell differentiation. At the end of T cell expansion, iPSC-derived T cells were analyzed by flow cytometry and an in vitro killing assay. As shown in FIG. 14A, the flow cytometry profiles of T cells generated from STTR2-generated, CAR-engineered iPSC clones showed homogenous expression of T identity markers (CD3ic and CD7). CAR expression was shown in >90% of the iPSC-derived T cells. Lack of TCR expression confirmed that T Cell Receptor Alpha Constant (TRAC) gene were disrupted by CRISPR engineering at the iPSC stage.

T cells differentiated from STTR2-generated, CAR-engineered iPSC were evaluated for their capacity to recognize and kill tumor target cells using a flow cytometry-based in vitro cytotoxicity assay. Primary CAR-T cells were included in the assay for comparison. The effector cells (primary CAR-T cells and iPSC CAR-T cells) were co-cultured with cancer cell lines with CAR-specific antigen expression (pos antigen tumor) and without CAR-specific antigen expression (neg antigen tumor) for about 4 hours with indicated effector to target (E:T) ratio and analyzed by flow cytometry. As shown in FIG. 14B, percent cytotoxicity for each effector to target (E:T) ratio was calculated according to the following formula: percent cytotoxicity=100−(% of remaining live target cells with test articles/% of remaining live target cells in absence of test articles×100). Collectively, the data show that T cells differentiated from STTR2-generated, CAR-engineered iPSC clones displayed similar antigen-specific killing functionality as compared to CAR-engineered primary T cells.

### Example 7—Transient and Temporal Reprogramming System For Generating Single Cell-Derived iPSC Bank as A Source of Derivative Cells For Therapeutic Uses

The STTR2 reprogramming and FMM2 maintenance compositions and methods have been used in-tandem to generate clonal master iPSC lines in this application for use as renewable and reliable cell sources for off-the-shelf immunotherapies. Donor-consented fibroblasts or T cells were transfected with the plasmid combination as disclosed. Reprogramming cells were sorted at clonal density into 96-well plates, and single cell-derived iPSC clones were expanded and screened for desired attributes including pluripotency, loss of reprogramming plasmids, genomic stability and differentiation potential. A selected clonal iPSC line was manufactured and cryopreserved under strict manufacturing and process quality controls, and the line was further subject to extensive characterization and testing in order to qualify as “master cell bank” as required under relevant regulation. iPSCs of the manufactured iPSC banks were differentiated following current good manufacturing practices into natural killer (NK) lineage or T lineage cells to a clinically relevant scale. The derivative cells were further subject to extensive characterization and testing in order to qualify as “drug substance and drug product” as required under relevant regulation. The iPSC-derived NK or T lineage cells were cryopreserved to generate a large number of doses at about, for example, 1×10⁸ cells/dose for use in adoptive cell therapy for blood and solid cancers as monotherapy or in combination with immune checkpoint inhibitors. Generally, 1×10⁸ cells/dose translates to 1.67×10⁶ cells/kg for a 60 kg patient. The dosage form, route of administration and dosing regimen for each indication were designed and determined according to preclinical data from GLP (Good Laboratory Practice) and non-GLP studies both in vitro and in vivo.

Beyond supporting iPSC-derived immune cells to treat cancer and immune diseases, footprint-free and feeder cell-free master iPSC lines generated by the STTR2 reprogramming platform and/or the FMM2 maintenance media have the potential to also enable off-the-shelf cell therapies for degenerative disorders, ranging from macular degeneration, diabetes, Parkinson's disease, blood disorders, to cardiovascular diseases.

One skilled in the art would readily appreciate that the methods, compositions, and products described herein are representative of exemplary embodiments, and not intended as limitations on the scope of the invention. It will be readily apparent to one skilled in the art that varying substitutions and modifications may be made to the present disclosure disclosed herein without departing from the scope and spirit of the invention.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure illustratively described herein suitably may be practiced in the absence of any element or elements, limitation or limitations that are not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A composition for induced pluripotent stem cell (iPSC) production comprising:
(i) a TGFβ family protein,
(ii) a ROCK inhibitor, and
(iii) a MEK inhibitor and a WNT activator,
wherein the composition does not comprise a TGFβ inhibitor,
wherein the composition is effective to improve iPSC pluripotency and genomic stability in a long-term iPSC maintenance.

2. The composition of claim 1,
(a) wherein the long-term iPSC maintenance comprises one or more of stages comprising: single cell dissociation of iPSC colonies, single cell sorting of dissociated iPSCs, iPSC single cell clonal expansion, clonal iPSC master cell bank (MCB) cryopreservation, thawing of iPSC MCB, and optionally additional cryopreserve-thaw cycles of the iPSC MCB; or
(b) wherein the TGFβ family protein is optionally added to the composition at single cell dissociation of iPSC colonies, or at iPSC single cell clonal expansion, or at any stage in-between; or
(c) wherein the MEK inhibitor and/or the WNT activator is at an amount 30-60% of that used in a reprogramming composition for reprogramming a non-pluripotent cell to the iPSC.

3. The composition of claim 1,
(i) wherein the TGFβ family protein comprises at least one of Activin A, TGFβ, Nodal, and functional variants or fragments thereof; and/or
(ii) wherein the WNT activator comprises a GSK3 inhibitor.

4. The composition of claim 2,
(i) wherein the non-pluripotent cell comprises a somatic cell, a progenitor cell, or a multipotent cell; or
(ii) wherein the non-pluripotent cell comprises a T cell; or
(iii) wherein the reprogramming composition comprises a ROCK inhibitor, a MEK inhibitor, a WNT activator, a TGFβ inhibitor, and optionally an HDAC inhibitor, wherein the TGFβ inhibitor and the HDAC inhibitor are included in the reprogramming composition at specific stages during reprogramming.

5. The composition of claim 1,
(i) wherein the improved long-term iPSC pluripotency is indicated by reduced pluripotency reversion or reduced spontaneous differentiation as compared to iPSCs without contact of the composition; and
(ii) wherein the improved genomic stability is indicated by a lower propensity for genomic abnormalities as compared to iPSCs without contact of the composition.

6. The composition of claim 5, wherein the improved genomic stability comprises reduction or prevention of trisomy or karyotype abnormality in iPSCs obtained from reprogramming a T cell.

7. The composition of claim 1, further comprising an iPSC, optionally wherein the iPSC comprises at least one genomic edit.

8. The composition of claim 2,
(i) wherein the iPSC maintenance further comprises iPSC genetic editing to obtain an engineered iPSC pool, single sell sorting of engineered iPSC pool, engineered iPSC single cell clonal expansion, clonal engineered iPSC master cell bank (MCB) cryopreservation, thawing of engineered iPSC MCB, and optionally additional cryopreserve-thaw cycles of the engineered iPSC MCB; and
(ii) wherein the engineered iPSC comprises at least one genomic edit.

9. A composition for induced pluripotent stem cell (iPSC) production comprising:
(i) a ROCK inhibitor, a MEK inhibitor, and a WNT activator;
(ii) an HDAC inhibitor; and
(iii) a TGFβ inhibitor,
wherein the composition is effective to improve reprogramming of a non-pluripotent cell to obtain iPSCs having established pluripotency and improved genomic stability, and optionally,
wherein, addition of (i), (ii) or (iii) to the composition is stage-specific during reprogramming of the non-pluripotent cell for an increased reprogramming efficiency.

10. The composition of claim 9,
(a) wherein the reprogramming of the non-pluripotent cell comprises one or more stages comprising: somatic cell transfection (day 0), exogenous gene expression, increase of heterochromatin, loss of somatic cell identity, and iPSC colony formation; or
(b) wherein the addition of the HDAC inhibitor is optionally at chromatin restructuring, or at around day 2-3 (post transfection); or
(c) wherein the addition of the TGFβ inhibitor is optionally at the loss of somatic cell identity, or at around day 6-8 (post transfection),
wherein the one or more stages in reprogramming is indicated by cell morphological change and/or marker gene profiling.

11. The composition of claim 9,
(i) wherein the HDAC inhibitor comprises valproic acid (VPA) or a functional variant or derivative thereof; and/or
(ii) wherein the WNT activator comprises a GSK3 inhibitor.

12. The composition of claim 9,
(i) wherein the non-pluripotent cell comprises a somatic cell, a progenitor cell, or a multipotent cell; or
(ii) wherein the non-pluripotent cell comprises a T cell.

13. The composition of claim 9,
(i) wherein the established pluripotency comprises a ground state pluripotency; and/or
(ii) wherein the established pluripotency is represented by increased naïve-specific gene expression comprising one or more of: MAEL, KLF4, DNMT3L, DPPA5, PRDM14, FGF4, UTF1, TFCP2L1, and TBX3; and/or
(iii) wherein the improved genomic stability is indicated by a lower propensity for genomic abnormalities than iPSCs obtained without contact of the composition during reprogramming; and/or
(iv) wherein the increased reprogramming efficiency is indicated by the higher percentage of cells expressing pluripotency maker genes in an iPSC pool after reprogramming than that of the iPSC pool obtained without contact of the composition during reprogramming.

14. A method of producing induced pluripotent stem cell (iPSC), comprising a step of cryopreserving a population of iPSCs,
wherein the iPSCs are in contact with the composition of any one of claims 1-8, and
wherein pluripotency and genomic stability of the iPSCs are maintained during cryopreservation; and optionally,
wherein the population of iPSCs comprising homogeneous iPSCs is expanded from a clonal iPSC single cell.

15. The method of claim 14, further comprising a step of expanding a single cell iPSC clone to obtain the population of clonal iPSCs,
wherein the iPSCs are in contact with the composition of any one of claims 1-8, and
wherein pluripotency and genomic stability of the iPSCs are maintained during expansion.

16. The method of claim 15, further comprising a step of single cell sorting of dissociated iPSCs to obtain a single cell iPSC clone,
wherein the iPSCs are in contact with the composition of any one of claims 1-8, and
wherein pluripotency and genomic stability of the iPSCs are maintained during single cell sorting.

17. The method of claim 16, further comprising a step of dissociating iPSC colonies to single cell iPSCs,
wherein the iPSCs are in contact with the composition of any one of claims 1-8, and
wherein pluripotency and genomic stability of the iPSCs are maintained during iPSC single cell dissociation.

18. The method of claim 17, further comprising a step of obtaining at least one colony comprising iPSCs generated from reprogramming a non-pluripotent cell.

19. The method of any one of claims 14-18, wherein the iPSCs are reprogrammed from a somatic cell, a progenitor cell, or a multipotent cell, or wherein the iPSCs are reprogrammed from a T cell.

20. The method of any one of claims 14-19,
(i) wherein the pluripotency comprises a ground state pluripotency; and/or
(ii) wherein the pluripotency is represented by increased naïve-specific gene expression comprising one or more of: MAEL, KLF4, DNMT3L, DPPA5, PRDM14, FGF4, UTF1, TFCP2L1, and TBX3; and/or
(iii) wherein the genomic stability comprises a lower propensity for genomic abnormalities than iPSCs in said step without contact of the composition.

21. A method of producing induced pluripotent stem cell (iPSC), wherein the method comprises:
(i) transferring to a non-pluripotent cell one or more reprogramming factors to initiate reprogramming of the cell; and
(ii) contacting the cell after step (i) with the composition of any one of claims 9-13 for a sufficient period, thereby generating at least one colony comprising iPSCs by reprogramming the non-pluripotent cell.

22. The method of claim 21, wherein the step of transferring comprises introducing to the non-pluripotent cell:
(i) one or more first plasmids, wherein each of the first plasmids comprises a replication origin, and a polynucleotide encoding one or more reprogramming factors, but does not comprise polynucleotides encoding an EBNA or a variant thereof; wherein the one or more first plasmids collectively comprise polynucleotides encoding at least OCT4, or at least OCT4, YAP1, SOX2 and large T antigen (LTag); wherein the introduction of one or more first plasmids induces a reprogramming process; and
(ii) one of: (1) a second plasmid comprising a nucleotide sequence encoding an EBNA, wherein the second plasmid does not comprise a replication origin or polynucleotide(s) encoding reprogramming factor(s); (2) an EBNA mRNA; and (3) an EBNA protein.

23. The method of claim 22, wherein the one or more first plasmids further collectively comprise polynucleotides encoding one or both of SOX2 and KLF, or one or more of MYC, LIN28, ESRRB and ZIC3.

24. The method of claim 21, wherein the step of contacting further comprises culturing the cells in presence of a ROCK inhibitor, a MEK inhibitor, a WNT activator, an HDAC inhibitor and a TGFβ inhibitor.

25. The method of claim 21, wherein the step of contacting comprises:
(a) contacting the cell after step (i) with a combination comprising a ROCK inhibitor, a MEK inhibitor, and a WNT activator, optionally at a stage of exogenous reprogramming factor expression, or at day 1-2 post reprogramming factor transferring (day 0);
(b) contacting the cell of step (a) with an HDAC inhibitor, optionally at a stage of chromatin restructuring, or at around day 2-3 post reprogramming factor transferring; and
(c) contacting the cell of step (b) with a TGFβ inhibitor, optionally at a stage of loss of somatic cell identity, or at around day 6-8 (post transfection),
thereby generating at least one colony comprising iPSCs;
wherein said stage is indicated by cell morphological change and/or marker gene profiling; and/or
wherein the iPSCs are footprint-free, have established pluripotency and improved genomic stability, and are produced with a higher efficiency as compared to reprogramming without steps (a), (b) and (c).

26. The method of claim 21,
(i) wherein the non-pluripotent cell comprises a somatic cell, a progenitor cell, or a multipotent cell; or
(ii) wherein the non-pluripotent cell comprises a T cell.

27. The method of claim 25,
(i) wherein the established pluripotency comprises a ground state pluripotency; and/or
(ii) wherein the established pluripotency is represented by increased naïve-specific gene expression comprising one or more of: MAEL, KLF4, DNMT3L, DPPA5, PRDM14, FGF4, UTF1, TFCP2L1, and TBX3; and/or
(iii) wherein the improved genomic stability comprises a lower propensity for genomic abnormalities than iPSCs from reprogramming without steps (a), (b) and (c); and/or
(iv) wherein the increased reprogramming efficiency is indicated by the higher percentage of cells expressing pluripotency marker genes in an iPSC pool after reprogramming than that of an iPSC pool obtained without contact of the composition during reprogramming.

28. The method of claim 26, wherein the improved genomic stability further comprises reduction or prevention of trisomy or karyotype abnormality in iPSCs obtained from reprogramming a T cell.

29. A method of producing induced pluripotent stem cell (iPSC), wherein the method comprises:
(i) transferring to a non-pluripotent cell one or more reprogramming factors to initiate reprogramming of the cell;
(ii) contacting the cell after step (i) with the composition of any one of claims 9-13 for a sufficient period, thereby generating at least one colony comprising iPSCs, wherein pluripotency and genomic stability of the iPSCs are established;
(iii) dissociating the iPSC colony of step (ii) to dissociated iPSCs, wherein the iPSCs are in contact with the composition of any one of claims 1-8;
(iv) sorting dissociated iPSCs to obtain one or more single cell iPSC clones, wherein the single cell iPSC clones are in contact with the composition of any one of claims 1-8; and optionally,
(v) expanding the single cell iPSC clone to a population of clonal iPSCs, wherein the population of clonal iPSCs is in contact with the composition of any one of claims 1-8; and optionally
(vi) cryopreserving the population of clonal iPSCs, wherein the cryopreserved population is in contact with the composition of any one of claims 1-8;
wherein pluripotency and genomic stability of the iPSCs are maintained during the step of dissociating, sorting, expanding, cryopreserving, or thawing.

30. The method of claim 29, wherein the method comprises cryopreserving the population of clonal iPSCs.

31. The method of claim 29, wherein the one or more reprogramming factors comprise at least OCT4.

32. The method of claim 29, wherein the step (i) of transferring comprises introducing to the non-pluripotent cell:
(a) one or more first plasmids, wherein each of the first plasmids comprises a replication origin, and a polynucleotide encoding one or more reprogramming factors, but does not comprise polynucleotides encoding an EBNA or a variant thereof; wherein the one or more first plasmids collectively comprise polynucleotides encoding at least OCT4, or at least OCT4, YAP1, SOX2 and large T antigen (LTag); wherein the introduction of one or more first plasmids induces a reprogramming process; and
(b) one of: (1) a second plasmid comprising a nucleotide sequence encoding an EBNA, wherein the second plasmid does not comprise a replication origin or polynucleotide(s) encoding reprogramming factor(s); (2) an EBNA mRNA; and (3) an EBNA protein.

33. The method of claim 32, wherein the one or more first plasmids further collectively comprise polynucleotides encoding one or both of SOX2 and KLF, or one or more of MYC, LIN28, ESRRB and ZIC3.

34. The method of claim 29, wherein the step (ii) of contacting further comprises culturing the cells in presence of a ROCK inhibitor, a MEK inhibitor, a WNT activator, an HDAC inhibitor and a TGFβ inhibitor.

35. The method of claim 29, wherein the step (ii) of contacting further comprises:
(a) contacting the cell after step (i) with a combination comprising a ROCK inhibitor, a MEK inhibitor, and a WNT activator, optionally at a stage of exogenous reprogramming factor expression, or at day 1-2 post reprogramming factor transferring (day 0);
(b) contacting the cell of step (a) with an HDAC inhibitor, optionally at a stage of chromatin restructuring, or at around day 2-3 post reprogramming factor transferring; and
(c) contacting the cell of step (b) with a TGFβ inhibitor, optionally at a stage of loss of somatic cell identity, or at around day 6-8 (post transferring), thereby generating at least one colony comprising iPSCs;
wherein said stage is indicated by cell morphological change and/or marker gene profiling; and/or
wherein the iPSCs have established pluripotency and improved genomic stability, and are produced with a higher efficiency as compared to reprogramming without steps (a), (b) and (c).

36. The method of claim 29, further comprising:
(1) contacting the cell of the sorting step (iv), expanding step (v), and cryopreserving step (vi), and optionally of the dissociating step (iii) with a ROCK inhibitor, a MEK inhibitor, and a WNT activator, wherein concentration of one or both of the MEK inhibitor and the WNT activator is 30%-60% of that in step (ii); and
(2) additionally contacting the cell of the expanding step (v) and cryopreserving step (vi), and optionally of the dissociating step (iii) and/or sorting step (iv) with a TGFβ family protein; and
wherein the cells in steps (iii), (iv), (v) and (vi) are not in contact with either a TGFβ inhibitor or an HDAC inhibitor.

37. The method of claim 29,
(i) wherein the non-pluripotent cell comprises a somatic cell, a progenitor cell, or a multipotent cell; or
(ii) wherein the non-pluripotent cell comprises a T cell.

38. The method of claim 29,
(i) wherein the pluripotency comprises a ground state pluripotency; and/or
(ii) wherein the pluripotency is represented by increased naïve-specific gene expression comprising one or more of: MAEL, KLF4, DNMT3L, DPPA5, PRDM14, FGF4, UTF1, TFCP2L1, and TBX3; and/or
(iii) wherein the iPSC comprises at least one genomic edit; and/or
(iv) wherein the genomic stability comprises a lower propensity for genomic abnormalities.

39. The method of claim 38, wherein the genomic stability further comprises reduction or prevention of trisomy or karyotype abnormality in iPSCs obtained from reprogramming a T cell.

40. The method of claim 29, wherein the method further comprises genetic editing of an iPSC to obtain an engineered iPSC pool, single sell sorting of engineered iPSC pool, engineered iPSC single cell clonal expansion, clonal engineered iPSC master cell bank (MCB) cryopreservation, thawing of engineered iPSC MCB, and optionally additional cryopreserve-thaw cycles of the engineered iPSC MCB; and wherein the engineered iPSC comprises at least one genomic edit.

41. A composition comprising an induced pluripotent cell (iPSC), a cell line, a clonal population or a master cell bank thereof, wherein the iPSC is contacted by a combination of a ROCK inhibitor, a MEK inhibitor, a WNT activator, and a TGFβ family protein, and wherein the iPSC comprises increased naïve-specific gene expression comprising one or more of: MAEL, KLF4, DNMT3L, DPPA5, PRDM14, FGF4, UTF1, TFCP2L1, and TBX3; and optionally the iPSC has at least one of the properties: high clonality, genetic stability, and ground state pluripotency.

42. The composition of claim 41, wherein the TGFβ family protein comprises at least one of Activin A, TGFβ, Nodal, and functional variants or fragments thereof; and/or wherein the WNT activator comprises a GSK3 inhibitor.

43. The composition of claim 41, wherein the iPSC is generated from reprogramming a non-pluripotent cell.

44. The composition of claim 43, wherein the non-pluripotent cell comprises a somatic cell, a progenitor cell, or a multipotent cell; or wherein the non-pluripotent cell comprises a T cell.

45. The composition of claim 44, wherein the iPSC comprises at least a genomic edit.

46. The composition of claim 41, further comprising a medium, wherein the medium is feeder-free.

47. The composition of claim 41, wherein the iPSC has at least one of the properties: high clonality, genetic stability, and ground state pluripotency.

48. An induced pluripotent cell (iPSC), a cell line, a clonal population or a master cell bank thereof produced by a method according to any one of claims 14-40.

49. The induced pluripotent cell (iPSC), a cell line, a clonal population or a master cell bank thereof of claim 48, wherein the iPSC comprises at least a genomic edit.

50. A derived non-natural cell or population thereof obtained from in vitro differentiation of the pluripotent cell or cell line of claim 48 or 49.

51. The derived non-natural cell or population thereof of claim 50, wherein the cell is an immune effector cell, and optionally, the immune effector cell comprises at least a genomic edit comprised in the iPSC.

52. The derived non-natural cell or population thereof of claim 50, wherein the cell comprises a CD34 cell, a hemogenic endothelium cell, a hematopoietic stem or progenitor cell, a hematopoietic multipotent progenitor cell, a T cell progenitor, an NK cell progenitor, a T cell, a NKT cell, an NK cell, a B cell, or an immune regulatory cell.

53. The derived non-natural cell or population thereof of claim 50, wherein the cell is a rejuvenated cell comprising at least one of the following properties: global increase of heterochromatin; improved mitochondrial function; increased DNA damage responses; telomere elongation and decrease of percentage of short telomere; decrease in the fraction of senescent cells; and higher potential for proliferation, survival, persistence, or memory like functions, in comparison to its natural cell counterpart.

54. A composition for use in manufacturing a pluripotent cell for application in cell-based therapies, wherein the composition comprises a pluripotent cell produced by a method according to any one of claims 14 to 40.

55. The composition of claim 54, wherein the pluripotent cell is allogeneic or autologous.

56. A kit for medicament use comprising a pluripotent cell obtained by a method according to any one of claims 14 to 40.

57. A kit for medicament use comprising the induced pluripotent cell of claim 48 or the derived non-natural cell of any one of claims 50 to 53.

58. An in vitro system for initiating reprogramming in a non-pluripotent cell, wherein the system comprises:
one or more first plasmids, wherein each of the first plasmids comprises a replication origin, and a polynucleotide encoding one or more reprogramming factors but does not encode an EBNA or a derivative thereof; wherein the one or more first plasmids collectively comprise polynucleotides encoding OCT4, YAP1, SOX2 and LTag; and optionally one of:
(1) a second plasmid comprising a nucleotide sequence encoding an EBNA, wherein the second plasmid does not comprise a replication origin or polynucleotide(s) encoding reprogramming factor(s);
(2) an EBNA mRNA; and
(3) an EBNA protein.

59. The system of claim 58, wherein the second plasmid has a high rate of loss; and wherein the expression of EBNA is transient and temporal.

60. The system of claim 58 or 59, wherein the system does not provide EBNA replication and/or continuous expression in the nucleus.

61. The system of any one of claims 58 to 60, wherein the system enables a transient/cytoplasmic expression of EBNA for a short duration, and prior to the appearance of pluripotency cell morphology and the induced expression of endogenous pluripotency genes.

62. The system of any one of claims 58 to 61, wherein the system enables a transient/cytoplasmic expression of one or more reprogramming factors comprised in the first plasmid(s) for a short duration, and prior to the appearance of pluripotency cell morphology and the induced expression of endogenous pluripotency genes.

63. The system of any one of claims 58 to 62, wherein the replication origin is selected from the group consisting of a Polyomavirinae virus, a Papillomavirinae virus, and a Gammaherpesvirinae virus.

64. The system of any one of claims 58 to 63, wherein the replication origin is one selected from the group consisting of SV40, BK virus (BKV), bovine papilloma virus (BPV), and Epstein-Barr virus (EBV).

65. The system of any one of claims 58 to 64, wherein the replication origin corresponds to, or is derived from, the wild-type replication origin of EBV.

66. The system of any one of claims 58 to 65, wherein the EBNA is EBV-based.

67. The system of any one of claims 58 to 66, wherein the one or more first plasmids further collectively comprise polynucleotides encoding reprogramming factor(s) comprising (i) one or more of NANOG, KLF, LIN28, MYC, ECAT1, UTF1, ESRRB, HESRG, CDH1, TDGF1, DPPA4, DNMT3B, ZIC3, and L1TD1; or (ii) MYC, LIN28, ESRRB, and ZIC3.

68. The system of any one of claims 58 to 67, wherein the polynucleotides encoding reprogramming factors are comprised in a polycistronic construct or non-polycistronic construct.

69. The system of claim 68, wherein the polycistronic construct comprises a single open reading frame or multiple open reading frames.

70. The system of any one of claims 58 to 69, wherein the system comprises two or more first plasmids, with each first plasmid comprising the same or different reprogramming factors encoded by at least one copy of the polynucleotide.

71. The system of any one of claims 58 to 70, wherein the system comprises four first plasmids, with each first plasmid comprising the same or different reprogramming factors encoded by at least one copy of the polynucleotide.

72. The system of any one of claims 58 to 71, wherein the system comprises four first plasmids, with each first plasmid comprising at least one copy of polynucleotides encoding OCT4 and YAP1, SOX2 and MYC, LIN28 and LTag, and ESRRB and ZIC3, respectively.

73. The system of any one of claims 58 to 72, wherein the first plasmid comprises more than one polynucleotide encoding reprogramming factors, wherein the adjacent polynucleotides are operatively connected by a linker sequence encoding a self-cleaving peptide or an IRES.

74. The system of claim 73, wherein the self-cleaving peptide is a 2A peptide, and is selected from the group comprising F2A, E2A, P2A and T2A.

75. The system of claim 74, wherein the 2A peptides comprised in the first plasmid constructs may be the same or different.

76. The system of claim 74 or 75, wherein two 2A peptides in neighboring positions are different.

77. The system of any one of claims 58 to 76, wherein the first and the second plasmids each comprise one or more promoters for expression of reprogramming factors and EBNA, and wherein the one or more promoters comprise at least one of CMV, EF1α, PGK, CAG, UBC, and other suitable promoters that are constitutive, inducible, endogenously regulated, or temporal-, tissue- or cell type-specific.

78. The system of any one of claims 58 to 77, wherein the first and the second plasmids each comprise a CAG promoter.

79. A kit comprising the system of any one of claims 58 to 78.
